(19) United States
(12) Patent Application Publication
(10) Pub. No.: US 2024/0151710 A1
(43) Pub. Date: May 9, 2024

(54) METHODS FOR DETECTING IMMUNE RESPONSE

(71) Applicants: MONASH UNIVERSITY, Clayton, Victoria (AU); ALFRED HEALTH, Melbourne, Victoria (AU); THE MACFARLANE BURNET INSTITUTE FOR MEDICAL RESEARCH AND PUBLIC HEALTH LTD, Melbourne, Victoria (AU)

(72) Inventors: MENNO VAN ZELM, Clayton (AU); ROBYN O'HEHIR, Melbourne (AU); MARK HOGARTH, Melbourne (AU)

(73) Assignees: MONASH UNIVERSITY, Clayton, Victoria (AU); ALFRED HEALTH, Melbourne, Victoria (AU); THE MACFARLANE BURNET INSTITUTE FOR MEDICAL RESEARCH AND PUBLIC HEALTH LTD, Melbourne, Victoria (AU)

(21) Appl. No.: 17/769,306

(22) PCT Filed: Oct. 16, 2020
(86) PCT No.: PCT/AU2020/051116

§ 371 Date: Apr. 14, 2022

(30) Foreign Application Priority Data
Oct. 17, 2019 (AU) 2019903919
Jun. 2, 2020 (AU) 2020901811

(51) Int. Cl. G01N 33/50; G01N 33/569; G01N 33/58; G01N 33/68

(52) U.S. Cl. CPC G01N 33/5052; G01N 33/56911; G01N 33/56983; G01N 33/582; G01N 33/6854; G01N 2800/24

(57) ABSTRACT

The invention relates to the field of medical diagnostics. In particular, it relates to compositions, methods and kits for detecting immune cells for diagnosis of allergy, for monitoring of vaccination responses, for determining immune response to pathogens and of treatment efficacy of allergen immunotherapy. For example, the invention provides a method of determining allergic reactivity in a subject, the method comprising, providing a sample from a subject, contacting the sample with a recombinant or synthetic allergen linked to a detectable label in conditions for permitting the binding of the allergen to an IgE molecule present in the sample, determining the binding of the allergen to an IgE molecule in the sample by detecting the label, wherein the detection of the label indicates the subject has allergic reactivity. For example, the invention provides method of detecting antigen-specific B cells in a subject, the method comprising: providing a sample from a subject, contacting the sample with an antigen linked to a detectable label in conditions for permitting the binding of the antigen to an Ig molecule on the surface of a B cell present in the sample, and determining the binding of the antigen to an Ig molecule in the sample by detecting the label, wherein the detection of the label indicates the subject has antigen-specific B cells.

### CROSS-REFERENCE TO EARLIER APPLICATIONS

This application claims priority from Australian provisional application nos. 2019903919 and 2020901811 the entire contents of each application is incorporated by reference in its entirety.

### FIELD OF THE INVENTION

The invention relates to the field of medical diagnostics. In particular, it relates to compositions, methods and kits for detecting immune cells for diagnosis of allergy, for determining immune response to pathogens, for monitoring of vaccination responses and of treatment efficacy of allergen immunotherapy.

### BACKGROUND OF THE INVENTION

The immune system can be activated in response to the presence of pathogens, such as virus, protozoa and bacteria, to the presence of vaccines and also to the presence of allergens. One aspect of the immune response is generation of immunoglobulins that are expressed or bound by certain cells. Detecting the presence of, for example, B-cells expressing immunoglobulins or basophils that bind immunoglobulins, can inform the immune status of an individual, identify immune-mediated disorders and inform on treatment options and response.

Allergic diseases are amongst the most common chronic immune-mediated disorders, and can manifest with enormous diversity in clinical severity and range of symptoms. As a result, there have been, and still are, major challenges in diagnosis, prediction of disease progression/evolution and treatment.

An allergic reaction in a subject is characterised by the induction of an immune response to innocuous antigen or allergen. The allergen triggers the activation of IgE-binding cells leading to a series of responses that are characteristic of allergy. Rapid and efficient detection of patients with allergies remains a challenge with current tests based on serum IgE levels, local allergic responses in the skin (skin prick test) and/or the presence of reactive IgE-loaded basophils in peripheral blood (basophil activation test (BAT)). Indeed, testing of patients via exposure to allergens, e.g. skin prick testing or food challenge can lead to adverse events. In many cases, testing requires close monitoring of subjects within a clinic. In contrast, tests based on allergen-specific IgE measurements, for example the RAST test, can be done using patient blood samples and are minimally invasive to the patient. However, a potential pitfall of these tests is the detection of ‘free’ serum IgE and not ‘functional’ IgE that is bound to effector cells, e.g. mast cells and basophils. The measurement of functional allergen-specific IgE can be performed by measuring basophil activation (BAT tests). In a BAT test, the patients' basophils are isolated and incubated for a short period of time (20 min-1 hr) with various potential allergens. The activation of basophils is then measured by histamine release or by cell surface expression of CD63 or upregulation of surface CD203c. While the BAT test is highly specific, there are pitfalls as patients with allergy can show a minor or no response. Moreover, the test is quite laborious for a cytometric test and it does not allow for component resolved diagnostics in a single assay. Thus, there is a need for new or improved methods for detecting patients who are at-risk of allergic responses.

Once diagnosed with an allergy to a specific allergen a patient may seek treatment to reduce the symptoms and reactions associated with the allergy. Traditionally, patients have been treated with one of two approaches: (1) symptoms being controlled through the pharmacologic neutralization of effector molecules, e.g. anti-histamines; and (2) the alteration of the immune response to the allergen via allergen immunotherapy (AIT). In AIT patients are exposed to allergens repeatedly over a period of time thereby altering their immune response. As patients are often required to be treated for several years for AIT to be effective, this can lead to reactions and difficulties in completing the therapy.

Over the past two decades, treatment of allergic diseases has dramatically changed with the introduction of therapeutics that target distinct pathways in allergy. These include monoclonal antibodies against IgE and type 2 cytokines and their receptors, and small molecule inhibitors of signal transduction pathways. Whilst therapeutics have shown some promise in modulating severe allergic responses, they are expensive, are not effective in all patients, and may require lifelong administration. The effectiveness of these therapies is all contingent on patients committing to long-term therapy with high costs.

With the current COVID-19 pandemic, the world is faced with an extreme situation of a highly infectious coronavirus (SARS-CoV-2 also known as 2019-nCoV) encountered by a global immunologically naïve population. COVID-19 infections globally have exceeded 36 million confirmed cases with more than 1 million deaths to date. The available PCR based test to detect SARS-CoV-2 RNA is the gold standard to confirm current infection. Disease severity ranges from mild to severe life-threatening with a substantial mortality rate. To manage the current situation and to expedite vaccine development, there is an urgent need to identify sensitive and specific immunological markers that demonstrate the generation of protective immune responses by the host. The presence or absence of these immune markers early after infection could be used to stratify patients with mild disease from those at risk of severe complications. The latter group could then be treated in early stages of infection, preventing disease or shortening hospitalisation. Furthermore, evidence of immunity in previously-infected healthcare workers can expedite their return to the frontline. Ultimately, comparison of immune markers between recovered patients and immunized individuals in vaccine trials could shed light on the functionality of the vaccine. This will be necessary to focus resources on the most promising candidates, and prevent delays in large-scale production for administration to people at risk for severe disease.

Accordingly, there is a need for diagnostic and prognostic methods to (1) diagnose allergies; (2) predict allergic disease progression in a patient; (3) define patients who would benefit from a specific immunotherapy treatment; (4) determine responders who are on an allergy treatment; (5) detect immune responses and memory to pathogens; and/or (6) monitoring of vaccination responses.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of determining allergic reactivity in a subject, the method comprising:

providing a sample from a subject,

contacting the sample with a natural, recombinant or synthetic allergen linked to a detectable label in conditions for permitting the binding of the allergen to an IgE molecule present in the sample, and

determining the binding of the allergen to an IgE molecule in the sample by detecting the label,

wherein the detection of the label indicates the subject has allergic reactivity.

In any aspect of the present invention, the allergen is recombinant or synthetic.

In any aspect of the present invention, the allergen is selected from a food-based allergen, airborne or environmental allergen, drug allergen, peptide allergen, goat milk allergen, plant allergen, animal allergen or arthropod allergen, preferably the arthropod is an insect, myriapod, arachnid or crustacean (for example, insect, mite, crustacean). Preferably, the allergen is a protein, and often an enzyme. Where the allergen is an enzyme, preferably the enzyme has been modified to reduce its activity, for example, the modification is a point mutation or truncation.

Suitable allergens include food-based allergens such as tree nuts, sesame, buckwheat, peanuts, milk proteins, egg whites, etc. Other allergens of interest include various airborne antigens, such as grass pollens, animal danders, house mite feces, etc., as well as insect venoms, and mold allergens. Typical food allergens include milk allergens (Bos d 4, 5 and 8), peanut allergens (Ara h 1, 2, 3, 6, 8 and 9), hazelnut (Cor a 9 and 14), cashew nut (Ana o 3), Walnut (Jug r 1), Brazil nut (Ber e 1), Sesame (Ses i 1), Buckwheat (Fag e 3), almond (Pru du 6), peach (Pru p 1 and Pru p 3), shrimp (Pen m 1) and wheat (Tri a 19; omega-5-gliadin). Common aeroallergens include Dermatophagoides pteryonyssinus (Der p 1 and 2); pollen allergens from ryegrass (Lol p 1, 5), timothy grass (Phl p 1, 5), Bahia grass (Pas n 1), Bermuda grass (Cyn d 1), ragweed (Amb a 1), pellitory species (Par o 1; Par j 1, 2), birch (Bet v 1) and other atmospheric pollens including Olea europaea, Artemisia sp., gramineae, etc.; and animal dander, e.g. from cats (Fel d 1) and dogs (Can f 1). Other allergens include venom allergens from the honeybee (Api m 1, 3, 10); phospholipases from the yellow jacket Vespula maculifrons and white faced hornet Dolichovespula maculate and venom from jumper ant Myrmecia pilosula. Other allergens of interest are those responsible for mould allergies (esp from the Alternaria, Aspergillus and Cladosporium species), as well as allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides. The specific allergen may be a polysaccharide, fatty acid moiety, protein, etc. In many cases the allergenic epitope is a polypeptide. Recombinant allergens may be produced by expression from recombinant DNA, obtained commercially or obtained by other techniques well-known in the art.

Typically, the recombinant allergen or antigen is linked to a tag that facilitates binding to the detectable label. For example, the tag may bind non-covalently to, or form a covalent interaction with, the detectable label. Suitable tags are known in the art and include the group consisting of streptavidin and derivatives thereof, avidin and derivatives thereof, biotin, immunoglobulins, monoclonal antibodies, polyclonal antibodies, recombinant antibodies, antibody fragments and derivatives thereof, leucine zipper domain of AP-1, jun, fos, hexa-his, hexa-hat glutathione S-transferase, glutathione affinity, Calmodulin-binding peptide, Strep-tag, Cellulose Binding Domain, Maltose Binding Protein, S-Peptide-Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, Con A or WGA and tetranectin or Protein A and Protein G. Most preferably, the tag is Bir-A. Even more preferably, the Bir-A tag is biotinylated.

In any aspect of the present invention, the detectable label is any moiety that allows detection. For example, the label may allow for colorimetric or fluorescent detection. Suitable fluorescent labels are known in the art and include fluorescein isothiocyanate (FITC), phycoerythrin (PE), peridin chlorophyll protein (PerCP), allophycocyanin (APC), Alexa fluor 488, Alexa 647, Alexa 710, Alexa fluor 405, cyanin 5 (Cy5), Cyanin 5.5 (Cy5.5), pacific blue (PacB), horizon violet 450 (HV450), pacific orange (Pac0), horizon-V500 (HV500), Krome Orange, Brilliant Violet 421 (BV421), Brilliant Violet 510 (BV510), Brilliant Violet 605 (BV605), Brilliant Violet 650 (BV650), Brilliant Violet 711 (BV711), Brilliant Violet 785 (BV785), Brilliant Ultraviolet 395 (BUV395), Brilliant Ultraviolet 496 (BUV496), Brilliant Ultraviolet 737 (BUV737), Orange Cytognos (00)515, quantum dots and conjugates thereof coupled with PE, to APC or to PerCP (e.g. PE/Cy5, PE/Cy5.5, PE/Cy7, PerCP/Cy5.5, APC/Cy7, APC-H7, APC-Alex750, PE-Texas Red, PE-Dazzle, PE-CF594) or any additional compatible fluorochrome or fluorochrome tandem. etc. A suitable label may be directly or indirectly linked to the recombinant allergen or antigen via the use of a suitable tag, including any tag described herein. In a preferred embodiment, the detectable label is linked to streptavidin. Where more than one allergen or antigen is being tested, each allergen or antigen may be linked to a different detectable label, i.e. each label is uniquely identifiable.

In any aspect of the invention, the recombinant allergen or antigen is linked to a biotinylated Bir-A tag which can bind to streptavidin linked to a detectable label. In this embodiment, the recombinant allergen or antigen is linked to a detectable label via the interaction between biotinylated Bir-A bound to streptavidin.

In any aspect of the invention, the allergen or antigen is linked to a tag or detectable label at the N-terminus of the allergen or antigen. Alternatively, the allergen or antigen is linked to a tag or detectable label at the C-terminus of the allergen or antigen

In any aspect of the invention, the sample is contacted with 2 or more, 3 or more, 4 or more, 5 or more, 6 or more, or 7 or more allergens or antigens each linked to a different detectable label.

In any aspect, the sample is contacted with the same allergen or antigen linked to 2 or more different labels (for example, an antigen linked to a first label and the same antigen linked to a separately detectable label), such as that shown in the Examples as double discrimination.

In any aspect of the invention, the sample may be a bodily fluid, for example a blood sample. Alternatively, the same may be a tissue sample. In one embodiment, the sample may contain a body fluid and a tissue sample. The blood sample may be a whole blood, buffy coat, peripheral blood mononuclear cell (PBMC), cord blood, purified or sorted cell population or bodily fluid. Bodily fluids include lymph, semen, nasal secretions, bronchial secretions, alveolar fluid, cerebrospinal fluid, endolymph, synovial fluid, pleural fluid, pericardial fluid (pericardial liquor), menstrual fluid, or combinations thereof. The tissue sample may be selected from tonsil, lymph node, bronchial, nasal or gut or skin biopsy.

In a preferred embodiment, the sample is a whole blood sample.

In any aspect, method comprises determining the binding of the allergen to an IgE molecule present on the surface of a cell in the sample.

In alternative embodiments, the sample is contacted with a bead, microparticle or chemical substrate to allow capture of a cell free IgE molecule. Alternatively, the sample is contacted with a tissue culture plate or chip of an array to allow capture of a cell free IgE molecule. The cell free IgE may be captured via any molecule known in the art or described herein that binds to IgE.

The IgE molecule may be present on the surface of an immune cell. Preferably, on the surface of an immune cell expressing an Fc epsilon receptor, FcεRI or FcεRII (CD23), such as a basophil, eosinophil, mast cell, monocyte, macrophage, B cell, activated T cell, platelet, follicular dendritic cell or thymic epithelial cell. Typically, the IgE molecule is present on the surface of a basophil. The IgE molecule is present on the surface of the basophil through binding to the high-affinity FcεRI.

In any aspect of the invention, the method further comprises removing cells present in the sample that do not have an IgE molecule on their surface bound to the allergen linked to a detectable label. Preferably, the IgE molecule on the surface of the cell is bound to FcεRI or FcεRII (CD23). For example, the removal of cells can be performed by electronic gating via sorting or following analysis by flow cytometric software to quantify the number of allergen bound cells.

In any aspect of the invention, the method further comprises removing the allergen linked to a detectable label that is not bound to IgE. Preferably, the allergen is removed by washing of the sample.

In any aspect of the invention, the method further comprises contacting the sample with a molecule that allows one or more, preferably two or more, immune cell types to be identified. Preferably, the molecule binds to an immune cell marker and allows detection. Typically, the molecule is linked to or is itself the detectable label. For example, the molecule may be a fluorescent dye, antibody, nucleotide probe or enzyme that leads to substrate being produced. Alternatively, the molecule is linked to a tag that facilitates binding to the detectable label. For example, the tag may bind non-covalently to, or form a covalent interaction with, the detectable label. Suitable tags are known in the art and have been described herein.

In a preferred embodiment, the molecule is an antibody that detects a marker of interest and the detectable label is a fluorochrome. Suitable fluorochromes are known in the art and have been described herein.

In further preferred embodiment, the marker of interest is a phenotypic marker used to identify a cell of interest, for example, a marker that allows identification of basophils. Preferably, the molecule distinguishes basophils from other cell types. For example, a pan-basophil marker may be used to identify basophils. Suitable basophil markers are CD63, IgE, 2D7 antigen, CD117, CD124, CD203c, CD200R3 or FcεRIα.

In any aspect of the invention, the recombinant allergen or antigen linked to a detectable label may be contacted with the sample prior to contacting the sample with a molecule that allows one or more, preferably two or more, immune cell types to be identified. Alternatively, the recombinant allergen or antigen linked to a detectable label may be contacted with the sample after contacting the sample with a molecule that allows one or more, preferably two or more, immune cell types to be identified.

In any aspect of the invention, the recombinant allergen or antigen linked to a detectable label may be contacted with the sample simultaneously to contacting the sample with a molecule that allows one or more, preferably two or more, immune cell types to be identified.

In any aspect of the invention, detecting a label, either linked to the recombinant or synthetic allergen, antigen or to the molecule, is performed by any method known in the art or described herein, including flow cytometry.

In any aspect of the invention, where an individual is determined by a method of the invention to have allergic reactivity, the method further comprises administering an allergy immunotherapy, preferably an allergen-specific immunotherapy, to the subject.

In another aspect, the present invention provides a method of detecting sensitization to an allergen in a subject, the method comprising:

providing a sample from a subject,

contacting the sample with a recombinant allergen linked to a detectable label in conditions for permitting the binding of the allergen to an IgE molecule present in the sample, and

determining the binding of the allergen to an IgE molecule in the sample by detecting the label,

wherein the detection of the label indicates the subject has been sensitized to the allergen.

In another aspect, the present invention provides a method of treating a subject identified as having allergic reactivity, the method comprising

determining whether the subject has allergic reactivity by performing or having performed a method as described herein; and

wherein if the subject has allergic reactivity then administering to the subject immunotherapy specific to the allergen they show reactivity to.

In another aspect, the present invention provides a method of treating a subject identified as having allergic reactivity, the method comprising

determining whether the subject has allergic reactivity by
providing or having provided a sample from a subject,
contacting or having contacted the sample with a recombinant or synthetic allergen linked to a detectable label in conditions for permitting the binding of the allergen to an IgE molecule present in the sample, and
determining or having determined the binding of the allergen to an IgE molecule in the sample by detecting the label,

wherein if the subject has allergic reactivity then administering to the subject immunotherapy specific to the allergen they are reactivity to.

In another aspect, the present invention provides a method of determining the efficacy of an allergy immunotherapy in a subject, the method comprising:

providing a first sample obtained from a subject before receiving allergy immunotherapy;

providing a second sample obtained from the subject during, or after, receiving allergy immunotherapy;

contacting the first and second samples from the subject with a recombinant allergen linked to a detectable label in conditions for permitting the binding of the allergen to an IgE molecule on the surface of a cell present in the samples; and

determining the binding of the allergen to an IgE molecule on the surface of a cell present in the first and second samples by detecting the label;

wherein a decrease in the total number of, or proportion of, IgE bound cells in the second sample relative to the first sample indicates efficacy of the allergy immunotherapy in the subject.

In any aspect, the allergy immunotherapy is allergen-specific immunotherapy.

In another aspect, the present invention provides a method of determining the efficacy of an allergy immunotherapy in a subject, the method comprising:

providing a first sample obtained from a subject before receiving allergy immunotherapy;

providing a second sample obtained from the subject during, or after, receiving allergy immunotherapy;

contacting the first and second samples from the subject with a recombinant allergen linked to a detectable label in conditions for permitting the binding of the allergen to an Ig molecule on the surface of a B cell present in the samples; and

determining the binding of the allergen to an Ig molecule on the surface of a B cell present in the first and second samples by detecting the label;

wherein an increase in the total number of, or proportion of, IgG-expressing B cells in the second sample relative to the first sample indicates efficacy of the allergy immunotherapy in the subject;

wherein an increase in the ratio of IgG: IgE-expressing B cells in the second sample relative to the first sample indicates efficacy of the allergy immunotherapy in the subject; or

wherein an increase in the total number of, or proportion of, IgG2 and/or IgG4-expressing B cells in the second sample relative to the first sample indicates efficacy of the allergy immunotherapy in the subject.

In one embodiment, the method further comprises contacting the first and second blood sample with a molecule that allows identification of B cells expressing IgG.

In another embodiment, the method further comprises contacting the first and second blood sample with a molecule that allows identification of B cells expressing IgM, IgA, IgG, IgD and IgE.

In this aspect, the method of the invention can be used for a wide variety of biological samples known or suspected to contain B cells. The sample may be blood, bone marrow or lymphoid tissue. The tissue may be selected from tonsil, lymph node, bronchial, nasal or gut biopsy. Alternatively, the blood sample may be a whole blood sample, buffy coat sample, peripheral blood mononuclear cell (PBMC) sample, cord blood, purified or sorted cell population or bodily fluid. Bodily fluids include samples from the group consisting of lymph amniotic fluid, nasal secretions, bronchial secretions, alveolar fluid, endolymph, pericardial fluid (pericardial liquor), peritoneal fluid, breast milk, or combinations thereof. Alternatively, the tissue may be selected from tonsil, lymph node, bronchial, nasal or gut biopsy.

In a preferred embodiment, the sample is a whole blood sample. In this aspect of the invention, the method may further comprise removing cells present in the sample that do not have surface Ig receptors bound to the allergen or antigen linked to a detectable label. For example, the removal of cells can be performed by electronic gating via sorting or following analysis by flow cytometric software to quantify the number of allergens or antigens bound per cell.

In one embodiment, the recombinant allergen or antigen linked to a detectable label that is not bound to surface immunoglobulin (Ig) on the B cell is removed. Preferably, the recombinant allergen or antigen is removed by washing of the sample.

In this aspect of the invention, the method further comprises contacting the sample with a molecule that allows one or more, preferably two or more, immune cell types to be identified. Preferably, the molecule binds to an immune cell marker and allows visible detection. Typically, molecule is bound or is itself the detectable label. For example, the molecule may be a fluorescent dye, antibody, nucleotide probe or enzyme that leads to substrate being produced. Alternatively, the molecule is linked to a tag that facilitates binding to the detectable label. For example, the tag may bind non-covalently to, or form a covalent interaction with, the detectable label. Suitable tags are known in the art and have been described herein.

In a preferred embodiment, the molecule is an antibody that detects a marker of interest and the detectable label is a fluorochrome. Suitable fluorochromes are known in the art and have been described herein.

In further preferred embodiment, the marker of interest is a phenotypic marker used to identify a cell of interest. Preferably, the molecule distinguishes B cells from other cells. For example, a pan-B cell marker may be used to identify B cells. Suitable B cell markers are CD19, CD20, CD79a or CD22. Preferably, the CD19 antigen. Fluorochrome-labelled antibodies for use in the recombinant or synthetic allergen or antigen, method or kit of the invention can be prepared according to routine techniques or they can be commercially obtained from various sources. Most preferably, the method comprises contacting the sample with labelled fluorochrome-conjugated antibodies directed to any one or more of markers: CD123, CD27, IgM, IgA, IgG, IgD, CD19, CD21, CD38 and IgE; preferably CD27, IgM, IgA, IgE, IgG, IgD, CD19, CD21 and CD38. The antibodies are provided with a detectable label, for example any detectable label as described herein. Typically, the detectable label allows separate detection and quantitation by flow cytometry.

In one aspect, the present invention provides a method of detecting antigen-specific B cells in a subject, the method comprising:

providing a sample from a subject,

contacting the sample with an antigen linked to a detectable label in conditions for permitting the binding of the antigen to an Ig molecule on the surface of a B cell present in the sample, and determining the binding of the antigen to an Ig molecule in the sample by detecting the label, wherein the detection of the label indicates the subject has antigen-specific B cells.

In one embodiment, the antigen is from a vaccine. In another embodiment, the antigen is not from a vaccine, for example it may be from an infectious agent or pathogen such as a virus, bacterium, fungi, protozoa or parasite.

Exemplary viruses include those that are associated with, or cause, respiratory conditions or diseases. The antigen-specific B cells may be specific for particular viral proteins, for example, nucleocapsid proteins (NCPs) or spike proteins, or domains within those proteins (e.g. S1 B). Exemplary viral proteins and domains include those defined herein, including the Examples.

The virus may be selected from: coronavirus, influenza, parainfluenza, respiratory syncytial virus (RSV), adenovirus, cytomegalovirus (CMV), Epstein-Barr virus (EBV), varicella zoster virus (VZV), dengue virus, rhinovirus, Herpes simplex virus and enteroviruses. More preferably, the virus is coronavirus or influenza. Even more preferably, the virus is severe acute respiratory syndrome coronavirus (SARS-CoV), Middle East respiratory syndrome coronavirus (MERS-CoV), or severe acute respiratory syndrome coronavirus 2 (SARS-CoV-2), most preferably SARS-CoV-2.

Exemplary bacteria are Clostridium tetani and Corynebacterium diphtheria, or bacteria that cause tetanus and diphtheria.

An exemplary parasite is one that caused malaria in humans. For example a parasite belonging to the genus Plasmodium (phylum Apicomplexa), in particular, P. falciparum, P. malariae, P. ovale, P. vivax and P. knowlesi.

In one embodiment, the antigen is an auto-antigen.

In another aspect the present invention provides a nucleic acid comprising a first nucleotide sequence encoding an allergen or antigen and a second nucleotide sequence encoding a tag. Preferably the allergen or antigen is any one described herein, including those listed in Table 1, for example SEQ ID NO: 7 to 51, 56 to 61, or 76-86. Nucleotides sequences encoding exemplary allergen or antigens are any as described herein, including those listed in Table 1, for example shown in SEQ ID NOs: 52 to 55, 62 to 71, or 87 to 90. Preferably the tag is any one described herein, including those listed in Table 1, for example SEQ ID NO: 4, 5, 6 or 72.

In another aspect the present invention provides a recombinant or synthetic polypeptide comprising an allergen or antigen and a tag, preferably the tag is any described herein. Preferably the allergen or antigen is any one described herein, including those listed in Table 1, for example SEQ ID NO: 7 to 51, 56 to 61, or 76-86. Preferably the tag is any one described herein, including those listed in Table 1, for example SEQ ID NO: 4, 5 or 6. Preferably the recombinant or synthetic polypeptide is obtained or obtainable by expression of a nucleic acid of the invention as described herein.

In another aspect the present invention provides a vector comprising a nucleic acid of the invention as described herein.

In another aspect the present invention provides a cell comprising a vector or nucleic acid of the invention as described herein.

The present invention provides a diagnostic or prognostic kit for comprising one or more recombinant allergens or antigens, a detectable label, together with instructions for use, buffer, and/or control samples. For example, provided is a diagnostic kit for use in any method described herein. Preferably, the recombinant allergen or antigen linked to a detectable label is suitable for use in flow cytometric immunophenotyping.

In any aspect, one or more or all of the steps of any method of the invention are performed in vitro or ex vivo. In one embodiment, in any method of the invention, the method does not include obtaining a sample from a subject.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic design of fluorescent antigen tetramer constructs. A. Construct design. The protein of interest with modification if needed to remove enzymatic activity and/or toxicity (*) with at the N-terminal end a signal peptide for periplasmic (bacterial) or extracellular localisation. In addition, the antigen construct contains a 6-histidine stretch (6His) for ease of purification with a cobalt column, as well as a BirA Substrate Peptide (LHHILDAQKMVWNHR) or (GLNDIFEAQKIEWHE) for targeted biotinylation. The 6His and BirA tags were either placed at the C-terminus, or in-between the N-terminal leader sequence and the antigen. B. Schematic of fluorescent antigen tetramer generation. The BirA sequence in the construct allows for in vitro targeted biotinylation. The biotinylated antigens can then be tetramerized through incubation with streptavidin. Streptavidin with a conjugated fluorochrome of choice can be used to obtain fluorescently-labelled antigen tetramers.

FIG. 2: Schematic of pipeline for production of fluorescent recombinant allergen or antigen tetramers. To maximize native folding and post-translational modification, one of three protein production systems are chosen: 1. E. coli BL21 (bacterial antigens; intracellular Eukaryotic antigens); 2. Sf21 insect cells (insect, mite, cockroach, plant allergens); and 3. Human 293Texpi cells (mammalian and viral antigens). Leader sequences direct the proteins to the periplasm (bacteria) or secretion into the culture supernatant (Sf21 and 293Texpi) from which the 6His-tagged proteins can be purified on a Cobalt (or nickel) column. Finally, purified proteins will be biotinylated in vitro with BirA enzyme, purified, and tetramerised with streptavidin conjugated to fluorochrome of choice.

FIG. 3: Detection of recombinant tetanus and diphtheria toxin proteins. A. Western blot using an anti-His detection antibody of recombinant TTC (tetanus toxin) and CRM197 (diphtheria toxin). Loaded are periplasm preps from E coli after induction of production, as well as purified fractions of these periplasm preps after enrichment for 6His-tag containing proteins on a cobalt-loaded retention column. Abbreviations: NR, non-reduced; R, reduced. B. Gating strategy for B-cells. C. Detection of tetanus specific B cells with TTC antigen tetramers (middle panel) and of diphtheria-specific B cells with CRM197 antigen tetramers (right panel). The use of two tetramers, each with a distinct fluorochrome increases sensitivity as small fractions of B cells express surface Ig that bind specifically to one of the fluorochromes (left panel). Abbreviations: NR, non-reduced; R, reduced.

FIG. 4: Recombinant influenza HA expression and use in detection of HA-specific IgG-antibodies in humans. A. Recombinant haemagglutinin (HA) proteins of the 2019 vaccine strains A/Michigan (AM15) and B/Phuket (BP13). Western blot using an anti-His detection antibody of recombinant AM15 and BP13. Loaded are non-reduced 293T expi culture supernatants enriched for 6His-tag containing proteins on a cobalt-loaded retention column. B. and C. IgG specific for AM15 and for BP13 in 16 healthy adults before and 4 weeks after vaccination with the 2019 quadrivalent influenza vaccine. All individuals had been vaccinated in 2018 as well. As the 2018 vaccine also contained AM15 and BP13, the 2019 vaccine was a booster vaccination for these 16 individuals. Statistics, paired t-test, ** p<0.01.

FIG. 5: Detection of recombinant allergen components. Western blots using an anti-His detection antibody of recombinant Api m 1 (predicted molecular weight (MVV), 20.3 kDa). Lol p 1 (MW: 31 kDa), Lol p 5 (MW: 33 kDa), Der p 2 (MW: 17 kDa), Fel d 1 (MW: 20.8 kDa), Ara h 2 (MW: 21.2 kDa), Pen m 1 (MW: 39 kDa) Loaded are culture supernatants of 293T expi cells (Fel d 1) or sf21 cells (all others) after enrichment for 6His-tag containing proteins on a cobalt-loaded retention column. MW predictions are based on denatured amino acid sequence and do not consider possible post-translational modifications (e.g. glycosylation). Abbreviations: NR, non-reduced; R, reduced.

FIG. 6: Flow-cytometric detection of basophil activation with streptavidin, allergen-tetramers and allergen extracts. A. Gating strategy for flow cytometric analysis of basophils following in vitro activation. Activated basophils are defined as being positive for surface CD63 expression. B. Frequencies of CD63 positive basophils of 24 controls (left panel) and 41 bee venom (BV)-sensitized individuals (right panel) following in vitro stimulation with either streptavidin-APC (negative control) or increasing concentrations of Api m 1 tetramerized with streptavidin-APC. C. Frequencies of CD63 positive basophils of 24 controls and 41 ryegrass pollen (RGP)-sensitized individuals following in vitro stimulation with either streptavidin-APC (negative control) or increasing concentrations of Lol p 1 tetramerized with streptavidin-APC. Abbreviation: APC, allophycocyanin. Each dot represents the measurement from an individual subject and lines connect the results from different stimuli performed on basophils from the same individual.

FIG. 7: Detection of allergen-specific IgE on blood basophils of allergic patients. A. Representative staining of basophils from blood of a bee venom (BV)-allergic patient stimulated with [Api m 1]₄-APC or streptavidin-APC—left plot. Median fluorescence intensity (MFI) of basophils from controls (middle) and BV-allergic subjects (right) following incubation with 1 μg/ml streptavidin-APC (“0”) or 0.01, 0.1 and 1 μg/ml [Api m 1]₄-APC. B. A) Left plot, representative staining of basophils from blood of a rye grass pollen (RGP)-allergic patient stimulated with [Lol p 1]₄-APC or streptavidin-APC. Median fluorescence intensity (MFI) of basophils from controls (middle) and RGP-allergic subjects (right) following incubation with 1 μg/ml streptavidin-APC (“0”) or 0.01, 0.1 and 1 μg/ml [Lol p 1]₄-APC. C) Staining intensities as ratio of [Api m 1]₄-APC/streptavidin-APC on basophils (left) and ROC curve of [Api m 1]₄ staining intensity (right). D) Staining intensities as ratio of [Lol p 1]₄-APC/streptavidin-APC on basophils (left) and ROC curve of [Lol p 1]₄ staining intensity (right). Statistics for allergen tetramer stains, Mann-Whitney U-test; for ROC curves, Wilson/Brown method to test whether the confidence level of the outcome distribution is greater than 95%. **** p<0.0001.

FIG. 8. Detection of a wide range of allergen components on blood basophils of allergic patients. A. Gating strategy for flow cytometric analysis of basophils. B-G. Representative staining of basophils from blood of relevant allergic patients with fluorescently-labelled allergen tetramers (solid lines) or fluorescently-labelled streptavidin (filled graphs). B. Lol p 5 staining on a ryegrass pollen allergic patient. C. Phl p 1 staining on a Timothy grass pollen allergic patient. D. Fel d 1 staining on a cat dander allergic patient. E. Der p 2 staining on a house dust mite allergic patient. F. Ara h 2 staining on a peanut allergic patient. G. Pen m 1 staining on a prawn allergic patient.

FIG. 9: Detection of allergen-specific B-cells. A. Gating strategy for B-cells. B. Detection of Api m 1 specific B cells with antigen tetramers (left panel). The use of two tetramers, each with a distinct fluorochrome increases sensitivity as small fractions of B cells express surface Ig that bind specifically to one of the fluorochromes (right panel). C. Detection of Lol p 1 specific B cells with antigen tetramers (left panel) and B cells from the same individual stained with fluorochrome-conjugated streptavidins only (right panel).

FIG. 10: Frequencies of Lol p 1 specific memory B cells (Bmem) expressing IgM, IgG, IgA or IgE. A. Flow cytometric data are gated using the strategy depicted in FIG. 10A. Dots represent individual data from 8 healthy controls and 42 ryegrass pollen (RGP)allergic individuals, red bars represent median values. Statistics, Mann-Whitney U test; **, p<0.01, ***, p<0.001, ns, not significant (p>0.05). Patients had significantly more IgG expressing Bmem than controls at the expense of IgM expressing Bmem B. and C. Frequencies of Lol p 1 specific B cells in RGP allergic patients out of the pollen season. Repeat samples were taken after 4 months, wherein 15 were treated with local symptom relief when needed and 15 received additional sublingual immunotherapy (SLIT) containing Sweet Vernal, Orchard, Perennial Rye, Timothy, and Kentucky Blue Grass Mixed Pollens Allergen Extracts. No differences were seen in the no SLIT group, whereas after 4 months of SLIT, there was a significant increase in IgM+ Bmem at the expense of IgG Bmem. Statistics, paired t test; *, p<0.05; **, p<0.01; ns, not significant (p>0.05).

FIG. 11: Immunophenotyping of Api m 1 specific B cells before and after ultra-rush immunotherapy. A. Stepwise gating of Api m 1 specific B cells from a bee venom sensitized patient before AIT with in left panel detection of Api m 1 specific B cells as in FIG. 8B, followed by a 2D plot (middle panel) to distinguish IgA+CD27+ memory B cells, followed by further subsetting of CD27+ IgA-cells to discriminate IgM+ memory and IgG+ memory B cells. B. The same analysis on the same patient, 2 weeks after start of ultra-rush bee venom AIT. C. Aggregate analysis of Api m 1 specific B cells from 3 bee venom sensitized individuals pre- and post-AIT. The distribution of B cells expressing distinct Ig isotypes was significantly different between the two timepoints with samples post-AIT having more IgG at the expense of IgM. Statistics, chi-square test; ****, p<0.0001.

FIG. 12: Immunophenotyping of vaccine-antigen specific B cells. A. Stepwise gating of CD3+ T cells and CD19+ B cells (left panel), followed by double discrimination of specific B cells specific to haemagglutinin (HA) of the H1N1 A/Michigan/2015 (AM15) strain. B. Subsetting of total B cells to discriminate naïve (IgM+CD27-) from memory B(mem) cells (all others; left panel), followed by separation of IgM/IgD+ unswitched Bmem from IgM-/IgD-switched Bmem. Withing switched Bmem, IgG1, IgG2, IgG3, IgG4 and IgA expression B cells can be distinguished. C. Subsetting of AM15-specific B cells with a similar approach as in B. D. Influenza booster vaccination results in increased numbers of AM15-specific Bmem cells, and E. through detailed immunophenotyping it was assessed that these specifically concerned IgG1+ Bmem. Statistics, Mann-Whitney U test; *, p<0.05; **, p<0.01.

FIG. 13: Detection of recombinant nucleocapsid and SIB proteins from SARS-CoV-2. A. Western blots using an anti-His detection antibody of nucleocapsid (left panel), 51B (middle panel) and 51 (right panel). Loaded are culture supernatants of 293T Expi cells after enrichment for 6His-tag containing proteins on a cobalt-loaded retention column. Calculated molecular weights of reduced proteins are −48 kDa (N), −30 kDa (51B) and 77 kDa (51). Abbreviations: NR, non-reduced; R, reduced. B. Serum IgG specific to antigens as determined by ELISA using the antigen to capture. 36 historic samples (collected in 2019 Q1 2020) of previously influenza-vaccinated healthy adults were run, as well as serum samples from 20 individuals after recovery from COVID-19 (convalescent). Statistics, Mann-Whitney U test; **** p<0.0001.

FIG. 14. Detection of B-cells with surface Ig specific for SARS-CoV2 nucleocapsid protein in a patient after recovery from COVID-19. 2D plots showing B cells of an uninfected control (A) and a recovered COVID-19 patient (B) stained with two fluorescent NCP tetramers conjugated to BUV395 and BUV737 and two fluorescent 51B tetramers conjugated to BV480 and BV650.

FIG. 15. CytoBas—Multiplex allergen stain to detect allergen sensitization on blood basophils using recombinant allergen tetramers. A. Using a 7-color flow panel, monoclonal antibodies CD123 and anti-IgE were used to identify blood basophils (CD123+ IgE+) and plasmacytoid dendritic cells (pDC; CD123+ IgE-), and on these cells, expression levels of IgE specific to Ara h 2, Fel d 1, Lol p 1, Lol p 5 and Api m 1 were determined. B. Expression levels of IgE and specific IgE to 5 allergens on basophils and pDC of 6 individuals previously diagnosed with a form of allergy. Patients 1 and 2 were diagnosed with ryegrass pollen allergy, patients 3 and 4 with bee venom allergy, patient 5 with cat dander allergy and patient 6 with peanut allergy. The ratio in median fluorescence intensity on (MFI) basophils (bold font) over MFI on pDC (regular font) can be used to quantify sensitization. This would omit the need for running a second staining with streptavidins to use as negative control.

FIG. 16. Immunophenotyping of COVID-19 specific B cells. A. Gating strategy for detection and subsetting of total B cells of COVID-19 patients into CD27-1gD+ naive, CD27+ IgD+ Bmem and IgD-Bmem. Within IgD-Bmem, distinct subsets were defined based on the differential expression of IgG1, 2, 3, 4 subclasses and IgA. B. Detection of NCP-specific B cells, and C. S1 B-specific B cells, followed by subsetting as in panel A. D. Relative compositions of Ig isotype an IgG subclass expressing Bmem within the total Bmem compartment, NCP+ Bmem and S1 B+ Bmem in 20 COVID-19 patients.

FIG. 17. Longevity of SARS-CoV2 specific Bmem. A. S1 B-specific IgG and NCP-spec IgG levels determined by ELISA using the recombinant protein constructs from FIG. 13A plotted versus time since symptom onset of 20 individuals, with 6 sampled twice. Paired samples are connected with black lines. B. S1B-specific and NCP-specific memory B cells (Bmem) detected with recombinant antigen tetramers (FIG. 16) and quantified per ml blood plotted versus time since symptom onset of 20 individuals, with 6 sampled twice. Paired samples are connected with black lines. C. IgM-expressing Bmem specific for S1B (left) and NCP (right), and D. IgG-expressing Bmem specific for S1 B (left) and NCP (right) depicted as in panel B. IgG+ Bmem of the 6 paired samples are significantly increased in the second sample; paired t test.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

As previously outlined above, current diagnosis testing for allergy requires either invasive intradermal challenge, in vitro culture of cells with allergens that may or may not be damaging to cells or enrichment of cells from blood prior to gain enough cells to detect the necessary immune cells. These limit the number of allergens that may be tested at a given time and/or require a much larger volume of blood to be drawn.

The present invention provides a means to diagnose allergy in a subject. The diagnosis utilises a sample of a patient e.g. blood without requiring enrichment of cells via magnetic means or culturing of cells with the allergen to induce activation. The diagnosis relies on differences in the amount of allergen that binds to cells through expressed or cell-bound immunoglobulins in allergic versus non-allergic subjects. In blood from subjects that are not sensitised to a specific allergen, cells expressing IgE or carrying cell-bound IgE do not bind this allergen. In blood from sensitised subjects, the IgE-expressing and IgE-carrying cells (esp basophils) may be labelled by the allergen. The presence of the labelled basophils is indicative of a hypersensitivity IgE response to that particular allergen. Additionally, the present invention can further be used to determine the efficacy of allergen immunotherapy (AIT). The effectiveness relies on changes in the frequency or portion of allergen-binding B cells, either IgE or IgG in subjects undergoing AIT therapy. The increase in the proportion of allergen-specific IgG-B cells, especially with respect to the frequencies of IgM-expressing and/or IgE-expressing B cells (either memory cells or plasma cells) is indicative of the therapy's effectiveness, as is the increase in the proportions of IgG2 and/or IgG4 within total IgG-expressing B cells.

The present inventors have developed methods that use recombinant allergens, which have been modified to reduce their toxicity but maintain their conformation. These recombinant allergens have been linked with a detectable label, for example in the form of a fluorescent molecule.

The present invention provides compositions and methods which allow diagnosis of allergen hypersensitivity in a subject or determination of the effectiveness of allergen immunotherapy. Advantages of aspects of the invention is that diagnosis of allergen hypersensitivity does not require the use of invasive intradermal challenge. Further, multiple allergens may be tested allowing the hypersensitivity of a subject to multiple allergens to be determined in a single assay, for example multiplexing in flow cytometry. Another advantage of aspects of the methods of the invention is that whole blood can be used avoiding the need for a step that processes or fractionates the blood. Further, there is no requirement to enrich the sample, for example, for a specific type of immune cell. Finally, as the method allows ex vivo analysis of a blood sample, there is no risk of systemic reaction with a positive result—contrast with food provocation, skin prick test or intradermal skin test.

The present invention also provides methods and compositions for monitoring of vaccination response, for example influenza, tetanus and diphtheria vaccines.

The present invention also provides methods and compositions for determining whether an individual has an immune response to an infectious agent, such as any described herein including a virus, a parasite or a bacterium. An advantage of the present invention is that it can allow identification of subjects who have received a vaccine or who have been naturally affected. For example, in the context of SARS-CoV-2, natural infection in a subject would result in separate populations of B cells specific to the nucleocapsid protein and specific to spike protein. However, many vaccine approaches do not include nucleocapsid proteins and therefore a subject that has had a natural infection would exhibit a population of B cells specific to the nucleocapsid protein. This can be applied to stratify patients for clinical trials.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Selected Definitions

As used herein, the term “allergen” refers to any naturally occurring protein or mixtures of proteins or chemicals/drugs that have been reported to induce allergic, i.e. IgE-mediated, reactions upon their repeated exposure to an individual.

An “allergy” also referred to herein as an “allergic reactivity,” is any condition where there is an undesired (e.g., a Type 1 hypersensitive) immune response (i.e., allergic response or reaction) to a substance. Such substances are referred to herein as allergens. Allergies or allergic conditions include, but are not limited to, allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. The allergic reaction may be the result of an immune reaction to any allergen. In some embodiments, the allergy is a food allergy. Food allergies include, but are not limited to, milk allergies, egg allergies, nut allergies, fish allergies, shellfish allergies, soy allergies or wheat allergies.

As used herein, the term “hypersensitivity” refers to an undesirable reaction produced by a normal immune response, including allergy and autoimmunity. This overreaction of the immune system may be damaging, uncomfortable or even fatal. Hypersensitivity reactions require a pre-sensitization of the host.

As used herein, the term “allergen sensitization” or “sensitization to an allergen” refers to the production of IgE antibodies following first exposure to an allergen or antigen that subsequently results in an allergic reaction or allergic reactivity.

As used herein, the term “antibody”, “immunoglobulin” or “Ig” refers to a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

As used herein, the term “immune cell” refers to any cell that is involved in an immune response. These cells include but are not limited to megakaryocyte, thrombocyte, erythrocyte, mast cell, myeloblast, basophil, neutrophil, eosinophil, monocyte, macrophage, dendritic cell, natural killer cell, NKT cells, NK-like cells, T cell, B cell and plasma cells.

As used herein, the term “FcεIR1+ immune cell” is intended to refer to a cell expressing the FcεRI high affinity receptor and which is capable of releasing a pharmacological mediator or mediators upon IgE-induced sensitization and exposure to an antigen of interest, such as mast cells and basophils.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of recombinant allergen or antigen, this term does not encompass a naturally-occurring allergen or antigen. However, if such an allergen or antigen is isolated, it is to be considered an isolated allergen or antigen. Similarly, if nucleic acid encoding the protein is isolated and expressed using recombinant means, the resulting protein is a recombinant allergen or antigen. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “binds” in reference to the interaction of an allergen or antigen with an antibody means that the interaction is dependent upon the presence of a particular structure (e.g., an antigenic determinant or epitope) on the allergen or antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “epitope” (syn. “antigenic determinant”) shall be understood to mean a region of a protein (such as an allergen or antigen) to which an antigen binding domain of an antibody binds.

As used herein, the term “condition” refers to a disruption of or interference with normal function, and is not to be limited to any specific condition, and will include diseases or disorders.

As used herein, the term “diagnosis” refers to the act of identifying a disease or condition using its signs or symptoms.

As used herein, the term “subject” shall be taken to mean any animal including humans, for example a mammal. Exemplary subjects include but are not limited to humans and non-human primates. For example, the subject is a human.

Allergy and Sensitization

It is understood that a method or composition of the present invention has several important applications, in particular in the area of medical diagnostics of humans.

Hypersensitivity reactions due to immunological responses can be classified into four broad classes. In particular, type I hypersensitivity reaction are those immediate type allergic reactions mediated by IgE-antibodies. In most allergies, such as those to food, pollen and house dust mites, reactions occur because the subject has become sensitised to an innocuous antigen—the allergen—by producing IgE antibodies against it. Subsequent exposure of the allergen triggers the activation of IgE binding cells, including mast cells and basophils, in the tissue or blood, leading to a series of responses that are characteristic of this type of reaction including degranulation of effector cells, release of histamines, heparin, eosinophil and neutrophil chemotactic factors, leukotrienes and thromboxane etc. Allergic immune responses are those characterised by the production of high levels of IgE antibody, which may be detected in the blood, and production of IgE-specific B cells.

The conventional tests for hypersensitivity include a skin prick test, where the allergen is injected intracutaneously or, occasionally, intradermally. A hypersensitivity or allergic response will cause rapid production of a wheal and erythema within 30 minutes. Other tests of allergy are known to the skilled person in the art and include immunoassays tests such as enzyme-linked immunosorbent assay (ELISA, or EIA) and radioallergosorbent test (RAST). The ELISA test measures the amount of allergen-specific antibodies in the blood and RAST test looks for specific allergen-related antibodies in order to identify your allergy triggers.

A test of the present invention may be used for diagnosis allergic reactivity or allergen sensitivity in circumstances where a skin prick test may not be warranted for example, (i) when the patient is using medicine known to interfere with the skin prick test such as antihistamines, steroids or certain antidepressants, (ii) the subject cannot tolerate many needle scratches that are required for the skin testing, (iii) the subject has an unstable heart condition, (iv) poorly controlled asthma, severe eczema, dermatitis, psoriasis or other severe skin condition and/or (v) might have an extreme reaction during skin testing or have a history of life-threatening allergic reactions e.g. anaphylaxis.

Types of allergies include but are not limited to food allergy, skin allergy, dust or pollen allergy, insect sting allergy, pet allergy, eye allergy, drug allergy, allergic rhinitis, latex allergy in particular Type I IgE-mediated allergic reaction, mold allergy, allergy associated sinus infection and cockroach allergy. Food allergies include but are not limited to allergies to milk, egg, peanut, three nut, soy, wheat, fish and shellfish. Drug allergies includes those which are IgE mediated by reacting to substances. The most common drug allergies include penicillin and other related antibiotics, antibiotics containing sulphonamides, anticonvulsants, aspirin, ibuprofen and other nonsteroidal anti-inflammatory drugs (NSAIDs) and chemotherapy drugs. The most common skin allergies include eczema (atopic dermatitis), hives (urticaria) and contact dermatitis. The most common forms of eye allergy are triggered by outdoor allergens, such as pollens from grass, trees and weeds, indoor allergens such as pet dander, dust mites and mold, irritants such as cigarette smoke, perfume and diesel exhaust. The most common dust or pollen allergy include dust mites, cockroaches, mold, pollen, pet hair, fur or feathers. The type of symptoms of an allergic reaction include but are not limited to mucus production, loss of sense of smell or taste, sore throat and/or cough, tiredness, temperature or shivers, facial congestion, headache, toothache, post nasal drip, wheezing, shortness of breath, trouble breathing, throat and mouth swelling, nausea, vomiting, bloating, diarrhea, stomach pain, cramping abdominal pain, skin rash, itching (in particular of the nose, eyes, ears and mouth), red and watery eyes, swelling around the eyes, hives, swelling of the lips, tongue or throat, high blood pressure, dizziness and/or fainting, severe asthma episode (asthma attack), chronic asthma and anaphylaxis

Allergens

Suitable allergens include food-based allergens such as tree nuts, sesame, buckwheat, peanuts, milk proteins, egg whites, shrimp etc. Other allergens of interest include various airborne antigens, such as grass pollens, animal danders, house mite feces, etc., as well as insect venoms, and mold allergens. Typical food allergens include milk allergens (Bos d 4, 5 and 8), peanut allergens (Ara h 1, 2, 3, 6 and 8), hazelnut (Cor a 9 and 14), cashew nut (Ana o 3), Walnut (Jug r 1), Brazil nut (Ber e 1), Sesame (Ses i 1), Buckwheat (Fag e 3), almond (Pru du 6), black tiger shrimp (Pen m 1) and wheat (Tri a 19). Common aeroallergens include Dermatophagoides pteryonyssinus (Der p 1 and 2); pollen allergens from ryegrass (Lol p 1, 5), timothy grass (Phl p 1, 5), Bahia grass (Pas n 1), Bermuda grass (Cyn d 1), ragweed (Amb a 1), pellitory species (Par o 1; Par j 1, 2), birch (Bet v 1) and other atmospheric pollens including Olea europaea, Artemisia sp., gramineae, etc.; and animal dander, e.g. from cats (Fel d 1) and dogs (Can f 1). Other allergens include venom allergens from the honey bee (Api m 1, 3, 10); phospholipases from the yellow jacket Vespula maculifrons and white faced hornet Dolichovespula maculate and venom from jumper ant Myrmecia pilosula. Other allergens of interest are those responsible for mould allergies (esp from the Alternaria, Aspergillus and Cladosporium species), as well as allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides. The allergen may be from a bacterium, for example, protein MGL_1304, which is secreted by the bacterium Malassezia (M.) globose, and is a major allergen for sweat allergy

The specific allergen may be a polysaccharide, fatty acid moiety, protein, etc. In many cases the allergenic epitope is a polypeptide. Recombinant allergens may be produced by expression from recombinant DNA, obtained commercially or obtained by other techniques well-known in the art.

The subject may be tested with one or a panel of suspected different allergens. The determination of the specific allergen to which a patient is hypersensitive allows the affected individual to seek treatment, e.g. desensitization, and to avoid activities that increase risk, e.g. exposure to the allergen. Panels may include a number of different pollens, groups of suspected food allergens, animal allergens, etc. The allergens may be multiplexed by the inclusion of different labels to the distinct allergens to allow facilitate detection. In one embodiment, the allergens and labels are those described in the Examples, including Example 6.

Typically, the recombinant or synthetic allergen or antigen is linked to a tag that facilitates binding to the detectable label. The tag may bind non-covalently to, or form a covalent interaction with, the detectable label. Suitable tags are known in the art and include the group consisting of streptavidin and derivatives thereof, avidin and derivatives thereof, biotin, immunoglobulins, monoclonal antibodies, polyclonal antibodies, recombinant antibodies, antibody fragments and derivatives thereof, leucine zipper domain of AP-1, jun, fos, hexa-his, hexa-hat glutathione S-transferase, glutathione affinity, Calmodulin-binding peptide, Strep-tag, Cellulose Binding Domain, Maltose Binding Protein, S-Peptid-Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C

Epitope, VSV Epitope, lectins that mediate binding to a diversity of compounds, including carbohydrates, lipids and proteins, Con A or WGA and tetranectin or Protein A and Protein G.

Most preferable the tag is Bir-A moiety which is subsequently biotinylated by contact with a Bir-A enzyme. The biotinylated recombinant allergens or antigens may be used in conjugation with any detectable label that can be linked by this moiety. The appropriate detectable label for each tag is known in the art.

The following is an example of a set of criteria for the design and production of recombinant or synthetic allergens or antigens:
1. The allergen or antigen should be non-pathogenic, non-toxic and not enzymatically active to minimize risks for researchers and to prevent cell death in target assays;
2. The allergen or antigen should be naturally folded and (if applicable) include post-translational modifications to ensure conformational epitopes are present;
3. The allergen or antigen should be able to be purified without affecting its protein structure; and
4. The allergen or antigen may contain a tag to facilitate conjugation to allow for multimerization and/or detection.

Recombinant or synthetic allergens can be produced based on any of the sequences from the WHO/IUIS Allergen Nomenclature database http://www.allergen.org. The sequences can be further modified by either substitution of one or more amino acids, deletion of one or more amino acids or addition of one or more amino acids. This can be achieved by techniques known to the skilled person in the art and those described herein. In particular, any mutation that conserves the structural conformation of the Ig binding motif is permitted and contemplated within the scope of the present invention.

For example, allergic sensitization to venom from the honey bee (Apis mellifera) venom and to ryegrass (Lolium perenne), their major allergens (Api m 1 and Lol p 1, respectively) were recombinantly produced based on the sequences from the WHO/IUIS Allergen Nomenclature database (http://www.allergen.org/). Both constructs contained the Api m 1 N-terminal leader sequence for extracellular production and C-terminal AviTag and 6-His sequences. Enzymatic activity was prevented through the introduction of point mutations: H34Q for Api m 1, (as described by Forster E et al. (1995) J Allergy Clin Immunol. 95(6):1229-35) and H104V for Lol p 1 (as described by Grobe K et al. (2002), Eur J Biochem 269(8):2083-92). Both constructs were codon optimized for Spodoptera frugiperda (fall armyworm) and cloned into the pFastBac vector (Thermo Fisher Scientific), prior to incorporation into a Bacmid for baculovirus expression.

Many allergens have enzymatic activity. Examples of enzymes are: Cysteine protease: Mite Group I allergens (Der p 1) and grass pollen group I allergens (Phl p 1, Lol p 1 etc); other proteases: Bumblebee Group IV (Bom p 4); Trypsin: Mites Group III (Der p 3); Chymotrypsin: Mites Group VI (Der p 6); Amylase: Mites Group IV (Der p 4); Nucleases: Grass pollen Group V (Phl p 5) and Tree pollen Group I (Bet v 1), Phospholipase: Insects Group I (Api m 1), Hyaluronidase: Insects Group II (Api m 2), Lysozyme: Chicken Group IV (Gal d 4) (as described by Bufe A (1998) Int Arch Allergy Immunol).

Enzymatic activity can be measured by an in vitro reaction through incubation of the purified protein with a substrate and performing kinetic measurements of substrate and/or product concentrations. For phospholipases (e.g. Api m 1), the substrate can be diC6thio-PM (racemic 2,3-bis(hexanoylthio)propyl-1-phosphomethanol lithium salt) or diC 6 thio-PC (1,2-dihexanoylthio-1,2-dideoxy-glycero-3-phosphosphocholine) and conversion of substrate to product can be measured using a spectrophotometer at 405 nm. Protease activity (e.g. Der p 1, Lol p 1) can be assessed by incubation of the purified protein for several hours at 37° C. and assessing its stability, e.g. by Western blotting.

Infectious Agents

The present invention finds particular application in the detection of antigen-specific B cells where the antigen is from an infectious agent such as a pathogen, for example a virus, bacterium, fungi, protozoa or parasite (or any pathogen or infectious agent described herein). Therefore, the present invention can be applied to determine the infection status and immunity towards a pathogen. The pathogen is not limited to human pathogens and includes other animal pathogens, for example, pathogens of cows, sheep, dogs, cats.

Exemplary viruses include those that are associated with, or cause, respiratory conditions or diseases. The antigen-specific B cells may be specific for particular viral proteins, for example, nucleocapsid proteins or spike proteins, or domains within those proteins. Exemplary viral proteins and domains include those defined herein, including the Examples.

The virus may be selected from: coronavirus, influenza, parainfluenza, respiratory syncytial virus (RSV), adenovirus, cytomegalovirus (CMV), Epstein-Barr virus (EBV), varicella zoster virus (VZV), dengue virus, rhinovirus, Herpes simplex virus and enteroviruses. More preferably, the virus is coronavirus or influenza. Even more preferably, the virus is severe acute respiratory syndrome coronavirus (SARS-CoV), Middle East respiratory syndrome coronavirus (MERS-CoV), or severe acute respiratory syndrome coronavirus 2 (SARS-CoV-2), most preferably SARS-CoV-2.

Exemplary bacteria are Clostridium tetani and Corynebacterium diphtheria, or bacteria that cause tetanus and diphtheria.

An antigen may be derived from any pathogen described herein, and may be referred to as a pathogen derived antigen.

Exemplary antigens (e.g. pathogen derived antigen) from various viral or bacterial infectious agents are described in Table 1.

Any reference to “allergen” as used herein, unless the context dictates otherwise, may be a reference to an “antigen”.

Auto-Antigens

Autoimmune diseases are broadly classified into two categories, organ-specific and systemic diseases. The precise aetiology of systemic auto-immune diseases is not identified. In contrast, organ-specific auto-immune diseases are related to a specific immune response including B and T cells, which targets the organ and thereby induces and maintains a chronic state of local inflammation. Examples of organ-specific auto-immune diseases include type 1 diabetes, myasthenia gravis, thyroiditis and multiple sclerosis. In each of these conditions, a single or a small number of auto-antigens have been identified, including insulin, the acetylcholine muscle receptor, thyroid peroxidase and major basic protein, respectively.

Autoimmune reactions are directed to a subjects own cells or tissues, more particularly to “auto-antigens” i.e. antigens (of proteins) that are naturally present in the subject. In this mechanism, auto-antigens are recognised by B- and/or T-cells which activate the immune system to attack the tissue comprising the auto-antigen.

Exemplary auto-antigens and diseases linked therewith, include thyroid diseases: thyroglobulin, thyroid peroxidase, and TSH receptor; Type 1 diabetes: insulin (proinsulin), glutamic acid decarboxylase (GAD), tyrosine phosphatase IA-2, heat-shock protein HSP65, islet-specific glucose6-phosphatase, catalytic subunit related protein (IGRP), adrenalitis: 21-OH hydroxylase; polyendocrine syndromes: 17-alpha hydroxylase, histidine decarboxylase tryptophan hydroxylase, tyrosine hydroxylase; gastritis & pernicious anemia: H+/K+ ATPase intrinsic factor; multiple sclerosis: myelin oligodendrocyte glycoprotein (MOG), myelin basic protein (MBP), and proteolipid protein (PLP); myasthenia gravis: acetyl-choline receptor; ocular diseases: retinol-binding protein (RBP); inner ear diseases: type II and type IX collagen; celiac disease: tissue transglutaminase: inflammatory bowel diseases: pANCA histone H1 protein: and atherosclerosis: heat-shock protein HSP60.

Detectable Labels

The present methods of the invention involve detection of immunoglobulins specific for a given allergen or antigen, for example IgE and IgG, using recombinant allergen or antigen linked to a detectable label.

The term “detectable” as used herein refers to an occurrence of, or a change in, a signal that is directly or indirectly detectable either by observation or by instrumentation. Typically, the detectable response is an occurrence of a signal wherein the fluorophore is inherently fluorescent. Alternatively, the detectable response is an optical response resulting in a change in the wavelength distribution patterns or intensity of absorbance or fluorescence or a change in light scatter, fluorescence lifetime, fluorescence polarization, or a combination of the above parameters.

Other detectable responses include, for example, chemiluminescence, phosphorescence, radiation from radioisotopes, magnetic attraction, and electron density.

The term “label,” as used herein, refers to a chemical moiety or protein that is directly or indirectly detectable (e.g. due to its spectral properties, conformation or activity) when attached to a recombinant or synthetic allergen or antigen and used in the present methods.

A detection label conjugated to allergen/antigen may be a fluorochrome. Suitable fluorescent labels are known in the art and include fluorescein isothiocyanate (FITC), phycoerythrin (PE), peridin chlorophyll protein (PerCP), allophycocyanin (APC), Alexa fluor 488, Alexa fluor 647, Alexa fluor 710, Alexa fluor 405, cyanin 5 (Cy5), Cyanin 5.5 (0y5.5), pacific blue (PacB), horizon violet 450 (HV450), pacific orange (Pac0), horizon-V500 (HV500), Krome Orange, Brilliant Violet 421 (BV421), Brilliant Violet 510 (BV510), Brilliant Violet 605 (BV605), Brilliant Violet 650 (BV650), Brilliant Violet 711 (BV711), Brilliant Violet 785 (BV785), Brilliant Ultraviolet 395 (BUV395), Brilliant Ultraviolet 496 (BUV496), Brilliant Ultraviolet 737 (BUV737), Orange Cytognos (00)515, quantum dots and conjugates thereof coupled with PE, to APC or to PerCP (e.g. PE/Cy5, PE/Cy5.5, PE/Cy7, PerCP/Cy5.5, APC/Cy7, APC-H7, APC-Alex750, PE-Texas Red, PE-Dazzle, PE-CF594) or any additional compatible fluorochrome or fluorochrome tandem, etc.

A suitable label may be directly or indirectly linked to the recombinant allergen/antigen via the use of a suitable tag. In a preferred embodiment, the detectable label is linked to streptavidin. Fluorochrome reagents are useful in panel reactivity assays, where a pool of two or more defined allergens/antigens are each conjugated to a different fluorochrome and added to a sample. A number of allergens/antigens may be tested at one time permitting multiplexing from a single blood draw. A blood sample is taken from a subject suspected of having a hypersensitivity or allergy to the test allergen.

Samples

Any biological sample that is known or suspected to contain a cell that displays an IgG (e.g. B cell) or IgE (e.g. basophil) is contemplated for use in the invention.

The term sample, as used herein, shall include blood samples but may also include hematopoietic biological samples such as lymph, leukopoiesis product, bone marrow and the like; also included in the term are derivatives and fractions of such fluids. The blood sample is drawn from any site for example by venepuncture. Blood samples will usually be from about 1 to 100 ml of whole blood, i.e. from 10⁵ to 10⁷ nucleated blood cells, and may be treated with anticoagulants, e.g. heparin, EDTA, citrate, acid citrate dextrose or citrate phosphate dextrose, as known in the art.

The sample may be a bodily fluid, for example a blood sample as discussed above. Alternatively, the same may be a tissue sample. The sample may contain a bodily fluid and a tissue sample.

The blood sample may be a whole blood, buffy coat, peripheral blood mononuclear cell (PBMC), cord blood, purified or sorted cell population or bodily fluid. Bodily fluids include lymph, semen, nasal secretions, bronchial secretions, alveolar fluid, cerebrospinal fluid, endolymph, synovial fluid, pleural fluid, pericardial fluid (pericardial liquor), menstrual fluid, or combinations thereof.

The tissue sample may be selected from tonsil, lymph node, bronchial, nasal or gut or skin biopsy. Preferably, the tissue sample is treated to form a single cell suspension. Forming a single cell suspension may be through a mesh filter for tonsil, thymus or lymph node. Alternatively, forming a single cell suspension may be via tissue digestion and then using a mesh through filter.

Specifically, in relation to biological samples known or suspected to contain B cells or basophils, the sample may be blood, bone marrow or lymphoid tissue. The tissue may be selected from tonsil, lymph node, bronchial, nasal or gut biopsy. Alternatively, the blood sample may be a whole blood sample, buffy coat sample, peripheral blood mononuclear cell (PBMC) sample, cord blood, purified or sorted cell population or bodily fluid. Bodily fluids include samples from the group consisting of lymph amniotic fluid, nasal secretions, bronchial secretions, alveolar fluid, endolymph, pericardial fluid (pericardial liquor), peritoneal fluid, breast milk, or combinations thereof.

The sample may be taken from any mammal including primate. In particular, human, murine, more particularly mouse, equine, bovine, ovine, porcine, canine, feline etc. Whole blood can be draft from the sample using any acceptable procedure. The use of whole blood allows detection of effector cells such as eosinophils and basophils. Alternatively, the blood samples may be resuspended in a solution that selectively lyses erythrocytes, e.g. ammonium chloride-potassium; ammonium oxalate, etc.

An advantage of the present invention is that the sample does not require any pre-processing and can be performed on the sample with minimal subsequent processing. However, in some circumstances the sample may be subjected to treatment such as dilution in buffered medium, concentration, filtration, or other gross treatment that will not involve the destruction of allergen/antigen-binding cells. Treatments may also include removal of cells by various techniques, including centrifugation, using Ficoll-Hypaque, panning, affinity separation, using antibodies specific for one or more markers present as surface membrane proteins on the surface of cells, or other techniques that provide for enrichment of leukocytes. For example, where a sample is diluted due to its large volume the sample may require concentration or centrifugation to allow a lower amount of recombinant allergen/antigen to be added.

The recombinant allergen/antigen as described above can be directly added to a whole blood sample. The amount of allergen/antigen necessary to bind a particular cell subset is empirically determined by performing a test assay. The amount may vary with the affinity of the allergen/antigen and the density of the specific binding partner e.g. member bound Ig or Ig bound to the surface Fc receptor. The cells and allergen/antigen are incubated for a period of time sufficient for the allergen/antigen to bind to either membrane bound Ig or Ig bound to the surface Fc receptor. This incubation time is usually at least about 10 minutes, not more than an hour, usually not more than 30 minutes.

Following incubation of the sample with a recombinant allergen or antigen linked to a detectable label the sample may be incubated with one or more molecules for the detection of immune cells. The molecule binds to an immune cell marker and allows, for example visible, detection. Typically, molecule is bound or is itself the detectable label. For example, the molecule may be a fluorescent dye, antibody, nucleotide probe or enzyme that leads to substrate being produced.

Alternatively, the molecule is linked to a tag that facilitates binding to the detectable label. For example, the tag may bind non-covalently to, or form a covalent interaction with, the detectable label. Suitable tags are known in the art and have been described herein. It is preferred that molecule is an antibody that detects a marker of interest, and the detectable label is a fluorochrome.

The recombinant or synthetic allergen/antigen linked to a detectable label may be contacted with the sample prior to contacting the sample with a molecule that allows one or more, preferably two or more, immune cell types to be identified. Alternatively, the recombinant allergen/antigen linked to a detectable label may be contacted with the sample after or at the same time as contacting the sample with a molecule that allows one or more, preferably two or more, immune cell types to be identified.

Detection Methods

The detection of immunoglobulins and/or immune cells, in particular B cells or basophils, binding recombinant or synthetic allergens/antigens linked to detectable labels may be performed by flow cytometry or microscopy. These methods are practiced as known in the art. The use of flow cytometry or microscopy may be used in conjugation with other cell phenotyping agents.

As an alternative to fluorescent detection, allergens or antigens can be multimerized, such as tetramerized, for example to streptavidins conjugated with isotopically pure elements for mass cytometry analysis. Using CyTOF (cytometry by time of flight) instruments, cells are nebulized and sent through an argon plasma, which ionizes the metal-conjugated antibodies. The metal signals are then analyzed by a time-of-flight mass spectrometer (for example, Spitzer et al (2016) Ce//165(4):780-9).

Where the sample is bound to a fluorochrome selective reagent is used, flow cytometry or microscopy may be used to detect the presence of immune cell labelled with the antigen/allergen conjugate. Such methods are practiced as known in the art. The method provides for the detection of allergen/antigen specific Ig either bound to immune cells or Ig expressed on the cell surface, for example B cells and basophils. In a non-allergenic patient sample, the number of allergen-binding cells in a sample may be low, due to the small number of cells in the starting population. In contrast, in an allergic patient test sample, the number of allergen binding cells present in a sample is usually around 20%, and in some cases as high as 90%. The purity may be evaluated by various methods. Conveniently, flow cytometry may be used in conjunction with light-detectable reagents specific for cell surface markers expressed by leukocytes. For diagnosis of allergy, the enriched cell population is analysed for the presence of allergen-binding cells, e.g. allergen-binding basophiles.

In an allergic patient, at least about 50% of the allergen-binding cells will be cells such as basophils, and may be as high as 90% of the allergen-binding cells. In a non-allergenic patient, less than about 10% of the allergen-binding cells are basophilic. A positive diagnosis of allergy to a specific allergen is made when the basophil population is increased relative to a control sample. The number of basophils may be at least about twice that of a normal, non-allergic donor in a similarly tested sample, and may be as high as about ten times the number of basophils in a control sample. Allergen/antigen-binding cells from the enriched cell population, particularly B cells from human donors, may be used to produce allergen/antigen-specific antibodies. The B cells may be immortalised through infection with Epstein-Barr virus, fusion with a myeloma cell line, transfection with a transforming retrovirus etc. Alternatively, the B cells may be sorted into a single cell well and the heavy chain and light chain amplified and sequenced. Antibodies from either the EBV-transformed cells or those produced via recombinant means can be screened by conventional methods e.g. ELISA, RIA, SPR etc. to determine the allergen specificity of the B cells that produce monoclonal IgE or IgG of particular interest for the production of testing reagents etc.

Detection of Immune Cells

The present invention is directed in part to the diagnosis of subjects with allergic reactivity or sensitization to an allergen. In particular, the method of the present invention facilitates detection of IgE-coated basophils in whole blood without the need for in vitro activation with allergens. Additionally, the present invention is also directed, in part, to determine the efficacy of allergen immunotherapy (AIT) or an allergy immunotherapy. In particular, the method of the present invention facilitates detection of allergen/antigen-specific B cells via binding of their Ig-surface receptor with the recombinant allergen/antigen. Comparison of the proportion of allergen-specific B cells with IgG will provide an indication of the efficacy of allergen therapy in the subject.

The method of the present invention further comprises contacting the sample with a molecule that allows one or more, preferably two or more, immune cell types to be identified. The molecule binds to an immune cell marker and allows visible detection. Typically, molecule is bound or is itself the detectable label. For example, the molecule may be a fluorescent dye, antibody, nucleotide probe or enzyme that leads to substrate being produced. Alternatively, the molecule is linked to a tag that facilitates binding to the detectable label. For example, the tag may bind non-covalently to, or form a covalent interaction with, the detectable label. Suitable tags are known in the art and have been described herein.

For example, the molecule is an antibody that detects a marker of interest and the detectable label is a fluorochrome. Suitable fluorochromes are known in the art and have been described herein. Several fluorescent conjugated antibodies directed to different phenotypic markers on immune cells may be added to the sample to facilitate the detection and discrimination of different cell types. Preferably, the sample is contacted with a panel of fluorochrome-conjugated antibodies under conditions suitable for antibody binding to their respective antigens.

The sample may be contacted with all the antibodies simultaneously, i.e. with a cocktail, mixture or composition of antibodies with or without the recombinant allergen/antigen. However, it may be suitable to add the antibodies in two or more steps. For example, a two-step incubation may be performed when both surface membrane and intracellular staining is necessary. In such cases, first the surface membrane staining is performed followed by fixation and permeabilisation to facilitate cytoplasmic staining. In each case, unlabelled antibodies may be used however; multiple incubations and wash steps may be required. Preferably, complex staining is not generally preferred in routine diagnostic testing.

CD stands for cluster designation and is a nomenclature for the identification of specific cell surface antigens defined by monoclonal antibodies. Antibodies against the indicated markers can be commercially obtained from various companies, including Becton Dickinson (BD) Biosciences, Dako, Beckman Coulter, CYTOGNOS, Caltag, Pharmingen, Exbio, Sanquin, Invitrogen, and the like.

Preferably, the antibodies are provided with a detectable label, which allows separate detection and quantitation, by flow cytometry. Numerous detectable fluorochrome labels are known in the art. For example, the panel of differentially-labelled antibody reagents comprises a combination of compatible fluorochromes selected from fluorescein isothiocyanate (FITC), phycoerythrin (PE), peridin chlorophyll protein (PerCP), allophycocyanin (APC), Alexa fluor 488, Alexa 647, Alexa 710, Alexa fluor 405, cyanin 5 (Cy5), Cyanin 5.5 (0y5.5), pacific blue (PacB), horizon violet 450 (HV450), pacific orange (Pac0), horizon-V500 (HV500), Krome Orange, Brilliant Violet 421 (BV421), Brilliant Violet 510 (BV510), Brilliant Violet 605 (BV605), Brilliant Violet 650 (BV650), Brilliant Violet 711 (BV711), Brilliant Violet 785 (BV785), Brilliant Ultraviolet 395 (BUV395), Brilliant Ultraviolet 496 (BUV496), Brilliant Ultraviolet 737 (BUV737), Orange Cytognos (00)515, quantum dots and conjugates thereof coupled with PE, to APC or to PerCP (e.g. PE/Cy5, PE/Cy5.5, PE/Cy7, PerCP/Cy5.5, APC/Cy7, APC-H7, APC-Alex750, PE-Texas Red, PE-Dazzle, PE-CF594) or any additional compatible fluorochrome or fluorochrome tandem. Fluorochrome-labelled antibodies can be used in the recombinant allergen/antigen, method or kit of the invention and can be prepared according to routine techniques known in the art, or obtained via various commercial sources.

In one example, the antibodies are conjugated to (1) pacific blue (PacB), brilliant violet 421 (BV421) or Horizon V450; (2) pacific orange (Pac0), Horizon V500 (HV500), BV510, Khrome orange (KO) or 00515, (3) Horizon BB515, fluorescein isothiocyanate (FITC) or Alexa488, (4) phycoerythrin (PE), (5) peridinin chlorophyl protein/cyanine 5.5 (PerCP-Cy5.5), PerCP or PE-TexasRed, (6) phycoerythrin/cyanine7 (PE-Cy7), (7) allophycocyanine (APC) or Alexa647, and (8) allophycocyanine/hilite 7 (APC-H7), APC-Cy7, Alexa680, APC-A750, APC-0750 or Alexa700.

In another example, the antibodies are conjugated to (1) brilliant violet 421, (2) brilliant violet 510 (BV510), (3) brilliant violet 650 (BV650), (4) brilliant violet 786 (BV786), (5) fluorescein isothiocyanate (FITC), (6) peridinin chlorophyl protein/cyanine 5.5 (PerCP-Cy5.5), (7) to phycoerythrin (PE), (8) phycoerythrin/cyanine7 (PE-Cy7), (9) allophycocyanine (APC), and (10) allophycocyanine/H7 (APC-H7), APC-0750 or APC-Alexa750.

Any suitable phenotypic marker can be used to identify a cell of interest. For example, a pan-basophil marker may be used to identify basophils. Suitable basophil markers are CD63, IgE, 2D7 antigen, CD117, CD124, CD203c, CD200R3 or FcεRIα. For example, a pan-B cell marker may be used to identify B cells. Suitable B cell markers are CD19, CD20, CD79a or CD22. Most preferably, the CD19 antigen.

It may be useful to include in the panel of fluorochrome-conjugated antibodies to further characterise the immune cells of interest, most preferably basophils and B cells. For example, the antibody is reactive with a marker for characterization of memory B cells, preferably a marker selected from the group consisting of CD23, CD40, CD80, CD86, CD180, TACI, CD200, CD73 and CD62L. TCL1 is suitably used for intracellular staining and discrimination between immature/naive versus memory B cells or for exclusion of FcεRII/CD23+ B-cells (e.g. CD23) that may bind IgE and appear as non-specifically stained IgE+ naive B-cells. The panel may also comprise one or more antibodies useful for the characterization of plasma B cells, for example an antibody against the CD20 or CD138 antigen. Additionally, further antibodies may be selected to characterise activated basophils: e.g. CD123, HLA-DR, CXCR3 and/or IgE.

For example, the sample is subjected to multi-color flow cytometry and gated for lymphocytes based on the forward scatter and side scatter, typically followed by exclusion of cell doublets and multiplets in an e.g. forward scatter-pulse area versus forward scatter pulse-height bivariate dot plot, according to conventional criteria.

Methods of Recombinant Protein Production

An allergen or antigen described herein may be recombinant or synthetic.

The present invention provides expression vectors and host cells transformed to express the nucleic acids of the invention. Exemplary methods, vectors and host cells are described in the Examples.

A nucleic acid sequence coding for a recombinant allergen or antigen of the invention or at least one fragment or portion thereof may be expressed in bacterial cells such as E. coli, insect cells (baculovirus), yeast, or mammalian cells such as Chinese hamster ovary cells (CHO) or human embryonic kidney cells (HEK 293T). Suitable expression vectors, promoters, enhancers, and other expression control elements may be found in Sambrook et al. Molecular Cloning: A Laboratory Manual, second edition, Cold Spring Harbor Laboratory Press, Cold Spring Harbor, N.Y. (1989). Other suitable expression vectors, promoters, enhancers, and other expression elements are known to those skilled in the art. Expression in mammalian, yeast or insect cells leads to partial or complete glycosylation of the recombinant material and formation of any inter- or intra-chain disulfide bonds. Suitable vectors for expression in yeast include YepSec 1 (Baldari C et al., (1987), EMBO J., 6(1): 229-234); pMFa (Kurjan and Herskowitz (1982), Cell, 30(3): 933-943); JRY88 (Schultz L D et al. (1987) Gene, 54(1): 113-123) and pYES2 (Invitrogen Corporation, San Diego, Calif.). These vectors are freely available. Baculovirus and mammalian expression systems are also available. For example, a baculovirus system is commercially available (PharMingen, San Diego, Calif.) for expression in insect cells while the pMSG vector is commercially available (Pharmacia, Piscataway, N.J.) for expression in mammalian cells.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1 a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Isolation of Proteins

Methods for isolating a protein are known in the art and/or described herein.

Where a recombinant allergen/antigen is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The recombinant allergen/antigen prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described.

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Nucleotide or Amino Acid Sequences

The present invention also contemplates modified forms of recombinant allergens/antigens of the invention comprising one or more conservative amino acid substitutions compared to a sequence set forth herein. In some examples, the recombinant allergen/antigen comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains 5 (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β--branched side chains (e.g., threonine, valine, isoleucine) and aromatic side chains (e.g., tyrosine, phenylalanine, tryptophan, histidine). Hydropathic indices are described, for example in Kyte and Doolittle (1982) J. Mol. Biol., 157 (1): 105-132 and hydrophilic indices are described in, e.g., U.S. Pat. No. 4,554,101.

Preferred nucleic acids encode a recombinant allergen/antigen having at least about 50% homology to a recombinant allergen/antigen of the invention, more preferably at least about 60% homology and most preferably at least about 70% homology with a recombinant allergen/antigen of the invention. Nucleic acids which encode recombinant allergen/antigen having at least about 90%, more preferably at least about 95%, and most preferably at least about 98-99% homology with a recombinant allergen/antigen of the invention are also within the scope of the invention. Homology refers to sequence similarity between two recombinant allergen/antigen or between two nucleic acid molecules. Homology can be determined by comparing a position in each sequence which may be aligned for purposes of comparison. When a position in the compared sequence is occupied by the same base or amino acid, then molecules are homologous at that position. A degree of homology between sequences is a function of the number of matching or homologous positions shared by the sequences.

The present invention also contemplates non-conservative amino acid changes. For example, of particular interest are substitutions of charged amino acids with another charged amino acid and with neutral or positively charged amino acids. In some examples, the recombinant allergen/antigen comprises 10 or fewer, e.g., 9 or 8 or 7 or 6 or 5 or 4 or 3 or 2 or 1 non-conservative amino acid substitutions.

In one example, the mutation(s) occur within the region that Ig binds to the recombinant allergen/antigen of the invention. In another example, the mutation(s) occur within the non-Ig binding portion of the allergen/antigen of the invention.

Exemplary methods for producing mutant forms of recombinant allergens/antigens include:
mutagenesis of DNA (Thie et al., (2009), Methods Mol. Biol. 525: 309-322) or RNA (Kopsidas et al., (2006) Immunol. Lett. 107(2):163-168; Kopsidas et al. (2007) BMC Biotechnology, 7: 18; and WO1999/058661);
introducing a nucleic acid encoding the polypeptide into a mutator cell, e.g., XL-1Red, XL-mutS and XL-mutS-Kanr bacterial cells (Stratagene);
DNA shuffling, (e.g., as disclosed in Stemmer, (1994) Nature 370 (6488): 389-91);
site directed mutagenesis, e.g., as described in Dieffenbach (ed) and Dveksler (ed) (In: PCR Primer: A Laboratory Manual, Cold Spring Harbor Laboratories, N Y, 1995);
H34Q mutation for Api m 1, (as described by Forster E et al. (1995) J Allergy Clin Immunol. 95(6):1229-35);
H104V mutations for Lol p 1 (as described by Grobe K et al. (2002) Eur J Biochem, 269(8):2083-92); and
Y98F mutations for HA proteins of H1N1 influenza strains to prevent sialic acid binding (as described by Whittle J R et al. (2014) Flow cytometry reveals that H5N1 vaccination elicits cross-reactive stem-directed antibodies from multiple Ig heavy-chain lineages. Journal of virology. 88(8):4047-4057.

Exemplary methods for determining biological activity of the mutant recombinant allergens/antigens of the invention will be apparent to the skilled artisan and/or described herein, e.g., recombinant allergens/antigens. For example, methods for determining allergen/antigen binding, competitive inhibition of binding, affinity, association, dissociation and therapeutic efficacy are described herein.

Monitoring Allergy Treatment Success

Methods of the present invention include monitoring or determining the success of allergy immunotherapy. For instance, the monitoring of treatment efficacy of anti-IgE therapy or allergy immunotherapy (oral/subcutaneous). The allergy immunotherapy may be allergen specific or non-allergen specific (e.g. omalizumab).

In particular, the invention relates to monitoring of the efficacy of allergy immunotherapy. Allergy immunotherapy (also termed hyposensitization therapy, immunologic desensitization, hyposensibilization, or allergen immunotherapy) includes immunotherapy for allergic disorders in which the patient is vaccinated with increasingly larger doses of an allergen with the aim of inducing immunologic tolerance. It also includes other treatments that are not allergen specific, for example those that reduce the sensitivity to allergens by targeting the IgE Fc region (e.g. omalizumab). Allergen specific immunotherapy is the only treatment strategy which treats the underlying cause of the allergic disorder. It can either reduce the need for medication, severity of symptoms or eliminate hypersensitivity altogether. Allergen can be administered under the tongue (sublingually), by injections under the skin (subcutaneous) or in some instances administered by intradermal injection.

The immune system of allergy affected individuals misinterprets a usually innocuous substance as a disease agent and begins producing IgE. This is called the ‘primary antibody response.’ The IgE produced during this response binds to basophils in the bloodstream and to a similar type of cell called mast cells in the tissues. When the person again encounters the allergen, these basophils and mast cells that have bound to IgE release histamine, prostaglandins, and leukotrienes, which causes inflammation of the surrounding tissues, resulting in allergic symptoms. Immunotherapy via repeated exposure to a specific allergen via either sublingual, subcutaneous intradermal, epicutaneous or intralymphatic route leads to a desensitisation to the allergen and thus a reduction in allergic symptomatology and use of symptomatic based treatments. The exact mechanism is not fully understood but it is accepted that immunotherapy causes modification of the immune system. This modification leads to changes in IgE synthesis and the production of IgE blocking antibodies which thus reduces the immune systems allergic response to specific allergens. There is also a shift from Th2 responses towards regulatory T cells. The molecular mechanism of such immunotherapy can be partly interpreted as that there occurs induction of allergen-specific IgG to neutralize the allergen instead of induction of allergen-specific IgE.

As will be appreciated by the skilled person, a method of the present invention is highly suitable to monitor any quantitative changes in allergen-specific B cells in a biological sample of a patient suffering from an allergy and/or receiving allergy immunotherapy. To that end, the IgG-expressing cells (be it memory B cells and/or plasma cells) are easily identified and quantitated by staining with the allergen of interest, the allergen being provided with a detectable label like a fluorochrome. Accordingly, provided is a method for the monitoring of treatment efficacy of allergy immunotherapy The method comprises analyzing memory B cell and plasma cell subsets in a biological sample isolated from a subject receiving said immunotherapy (oral/subcutaneous) using a procedure as described herein above for detecting allergen IgG+ memory B cells and/or IgG+ plasma cells. The procedure includes determining the allergen specificity of the IgG+ memory B cell population and/or the IgG+ plasma cell population by contacting the cells with a fluorochrome-conjugated allergen of interest and further comprises the step of correlating the amount of IgG+ memory B cells and/or IgG+ plasma cells with the disease diagnosis and/or classification, wherein an increase in the number of allergen-specific IgG+ memory B cells and/or IgG+ plasma cells as compared to pre-treatment values is indicative of the treatment being successful.

A positive response to allergy immunotherapy may be any one or more of (where the first sample is prior to immunotherapy and the second is during, or after, immunotherapy):

an increase in the total number of, or proportion of, IgG-expressing B cells in the second sample relative to the first sample;

an increase in the ratio of IgG: IgE-expressing B cells in the second sample relative to the first sample; or

an increase in the total number of, or proportion of, IgG2 and/or IgG4-expressing B cells in the second sample relative to the first sample; or

a decrease in the total number, or proportion of, basophils having bound IgE in the second sample relative to the first sample.

An increase or decrease in the total number of, or proportion of, an Ig may be determined by an increase or decrease in the intensity of the detectable label, e.g. i.e. the amount of allergen/antigen linked detectable label molecules that bind per cell.

Kits

A kit may be provided for the practice of the subject invention. For example, the kit may include one or a panel of recombinant allergens/antigens linked to a detectable label as described herein, and, optionally, other antibodies that phenotype the cells of interest. Optionally, a kit of the invention is packaged with instructions for use in a method described herein.

A still further aspect of the invention relates to a diagnostic kit comprising reagents for performing a method herein disclosed. In one embodiment, it is a diagnostic assay kit e.g. to diagnose and/or classify and/or monitor treatment efficacy of a disease or condition associated with altered (production of) IgG levels and/or IgE specificity. The kit comprises a panel of fluorochrome-conjugated antibodies against IgM, IgA, IgG, IgD and IgE; an antibody against a B cell marker and an antibody against the CD38 antigen. Preferably, the B cell marker is CD19, CD20, CD79a or CD22 antigen, more preferably CD19 antigen. The kit may also contain a fluorochrome-conjugated CD27 antibody. Each antibody may be conjugated to a distinct fluorochrome to allow for distinct detection by flow-cytometry.

The kit may further comprise any additional reagent, buffer, or device for use in a method of the invention. For example, it may contain reagents to prepare a standard curve, to calibrate the flow cytometer, positive controls, negative controls, and the like.

TABLE 1
Summary of exemplary amino acid
and nucleotide sequences.

| | | SEQ | Amino acid sequence/ |
|---|---|---|---|
| Type of | Sequence | ID | Nucleotide |
| sequence | description | NO: | sequence |
| Leader | Bacteria | 1 | MIKFLSALILLLVTTAAQA |
| sequence | ssFlg | | |
| | leader | | |
| | Insect | 2 | MQVVLGSLFLLLLSTSHGWQ |
| | Api m | | IRDRIGDNELEER |
| | 1 leader | | |
| | Mammal Ig | 3 | MVLSLLYLLTALPGILS |
| | leader | | |
| Tag | 6His | 4 | HHHHHH |
| | Tag | | |
| | BirA | 5 | LHHILDAQKMVWNHR |
| | Tag 1 | | |
| | BirA | 6 | GLNDIFEAQKIEWHE |
| | Tag 2 | | |
| vaccine | TTC | 7 | KNLDCWVDNEEDIDVILKKS |
| antigen | | | TILNLDINNDIISDISGFNS |
| | | | SVITYPDAQLVPGINGKAIH |
| | | | LVNNESSEVIVHKAMDIEYN |
| | | | DMFNNFTVSFWLRVPKVSAS |
| | | | HLEQYDTNEYSIISSMKKYS |
| | | | LSIGSGWSVSLKGNNLIWTL |
| | | | KDSAGEVRQITFRDLSDKFN |
| | | | AYLANKWVFITITNDRLSSA |
| | | | NLYINGVLMGSAEITGLGAI |
| | | | REDNNITLKLDRCNNNNQYV |
| | | | SIDKFRIFCKALNPKEIEKL |
| | | | YTSYLSITFLRDFWGNPLRY |
| | | | DTEYYLIPVAYSSKDVQLKN |
| | | | ITDYMYLTNAPSYTNGKLNI |
| | | | YYRRLYSGLKFIIKRYTPNN |
| | | | EIDSFVRSGDFIKLYVSYNN |
| | | | NEHIVGYPKDGNAFNNLDRI |
| | | | LRVGYNAPGIPLYKKMEAVK |
| | | | LRDLKTYSVQLKLYDDKDAS |
| | | | LGLVGTHNGQIGNDPNRDIL |
| | | | IASNWYFNHLKDKTLTCDWY |
| | | | FVPTDEGWTND |
| | CRM197 | 8 | GADDVVDSSKSFVMENFSSY |
| | | | HGTKPGYVDSIQKGIQKPKS |
| | | | GTQGNYDDDWKG(E)FYSTD |
| | | | NKYDAAGYSVDNENPLSGKA |
| | | | GGVVKVTYPGLTKVLALKVD |
| | | | NAETIKKELGLSLTEPLMEQ |
| | | | VGTEEFIKRFGDGASRVVLS |
| | | | LPFAEGSSSVEYINNWEQAK |
| | | | ALSVELEINFETRGKRGQDA |
| | | | MYEYMAQACAGNRVRRSVGS |
| | | | SLSCINLDWDVIRDKTKTKI |
| | | | ESLKEHGPIKNKMSESPNKT |
| | | | VSEEKAKQYLEEFHQTALEH |
| | | | PELSELKTVTGTNPVFAGAN |
| | | | YAAWAVNVAQVIDSETADNL |
| | | | EKTTAALSILPGIGSVMGIA |
| | | | DGAVHHNTEEIVAQSIALSS |
| | | | LMVAQAIPLVGELVDIGFAA |
| | | | YNFVESIINLFQVVHNSYNR |
| | | | PAYSPGHKTQPFLHDGYAVS |
| | | | WNTVEDSIIRTGFQGESGHD |
| | | | IKITAENTPLPIAGVLLPTI |
| | | | PGKLDVNKSKTHISVNGRKI |
| | | | RMRCRAIDGDVTFCRPKSPV |
| | | | YVGNGVHANLHVAFHRSSSE |
| | | | KIHSNEISSDSIGVLGYQKT |
| | | | VDHTKVNSKLSLFFEIKS |
| | HBsAg | 9 | ENITSGFLGPLLVLQAGFFL |
| | | | LTRILTIPQSLDSWWTSLNF |
| | | | LGGSPVCLGQNSQSPTSNHS |
| | | | PTSCPPICPGYRWMCLRRFI |
| | | | IFLFILLLCLIFLLVLLDYQ |
| | | | GMLPVCPLIPGSTTTSTGPC |
| | | | KTCTTPAQGNSMFPSCCCTK |
| | | | PTDGNCTCIPIPSSWAFAKY |
| | | | LWEWASVRFSWLSLLVPFVQ |
| | | | WFVGLSPTVWLSAIWMMWYW |
| | | | GPSLYSIVSPFIPLLPIFFC |
| | | | LWVYI |
| | HA_A- | 10 | MKAILVVLLYTFTTANADTL |
| | Michigan- | | CIGYHANNSTDTVDTVLEKN |
| | 2015 | | VTVTHSVNLLEDKHNGKLCK |
| | | | LRGVAPLHLGKCNIAGWILG |
| | | | NPECESLSTASSWSYIVETS |
| | | | NSDNGTCY(F)PGDFINYEE |
| | | | LREQLSSVSSFERFEIFPKT |
| | | | SSWPNHDSNKGVTAACPHAG |
| | | | AKSFYKNLIWLVKKGNSYPK |
| | | | LNQSYINDKGKEVLVLWGIH |
| | | | HPSTTAD |
| | | | QQSLYQNADAYVFVGTSRYS |
| | | | KKFKPEIATRPKVRDREGRM |
| | | | NYYWTLVEPGDKITFEATGN |
| | | | LVVPRYAFTMERNAGSGIII |
| | | | SDTPVHDCNTTCQTPEGAIN |
| | | | TSLPFQNIHPITIGKCPKYV |
| | | | KSTKLRLATGLRNVPSIQSR |
| | | | GLFGAIAGFIEGGWTGMVDG |
| | | | WYGYHHQNEQGSGYAADLKS |
| | | | TQNAIDKITNKVNSVIEKMN |
| | | | TQFTAVGKEFNHLEKRIENL |
| | | | NKKVDDGFLDIWTYNAELLV |
| | | | LLENERTLDYHDSNVKNLYE |
| | | | KVRNQLKNNAKEIGNGCFEF |
| | | | YHKCDNTCMESVKNGTYDYP |
| | | | KYSEEAKLNREKIDGSGYIP |
| | | | EAPRDGQAYVRKDGEWVLLS |
| | | | TFLGS |
| | HA_B- | 11 | MKAIIVLLMVVTSNADRICT |
| | Phuket-2013 | | GITSSNSPHVVKTATQGEVN |
| | | | VTGVIPLTTTPTKSYFANLK |
| | | | GTRTRGKLCPDCLNCTDLDV |
| | | | ALGRPMCVGTTPSAKASILH |
| | | | EVRPVTSGCFPIMHDRTKIR |
| | | | QLPNLLRGYEKIRLSTQNVI |
| | | | DAEKAPGGPYRLGT(G)SGS |
| | | | CPNATSKIGFFATMAWAVPK |
| | | | DNYKNATNPLTVEVPYICTE |
| | | | GEDQITVWGFHSDNKTQMKS |
| | | | LYGDSNPQKFTSSANGVTTH |
| | | | YVSQIGDFPDQTEDGGLPQS |
| | | | GRIVVDYMMQKPGKTGTIVY |
| | | | QRGVLLPQKVWCASGRSKVI |
| | | | KGSLPLIGEADCLHEEYGGL |
| | | | NKSKPYYTGKHAKAIGNCPI |
| | | | WVKTPLKLANGTKYRPPAKL |
| | | | LKERGFFGAIAGFLEGGWEG |
| | | | MIAGWHGYTSHGAHGVAVAA |
| | | | DLKSTQEAINKITKNLNSLS |
| | | | ELEVKNLQRLSGAMDELHNE |
| | | | ILELDEKVDDLRADTISSQI |
| | | | ELAVLLSNEGIINSEDEHLL |
| | | | ALERKLKKMLGPSAVDIGNG |
| | | | CFETKHKCNQTCLDRIAAGT |
| | | | FNAGEFSLPTFDSLNITGSG |
| | | | YIPEAPRDGQAYVRKDGEWV |
| | | | LLSTFLGS |
| Insect venom | Api m 1 | 12 | APPKAVLKLEPPWINVLRED |
| allergen | | | SVTLTCGGAHSPDSDSTQWF |
| | | | HNGNLIPTHTQPSYRFKANN |
| | | | NDSGEYRCQTGRTSLSDPIH |
| | | | LTVLSEWLALQTPHLEFREG |
| | | | ETIMLRCHSWKDKPLIKVTF |
| | | | FQNGISKKFSH(Q)MDPNFS |
| | | | IPQANHSHSGDYHCTGNIGY |
| | | | TPYSSKPVTITVQVPSVGSS |
| | | | SP |
| | Api m 10 | 13 | ERDQMMAATFDFPSLSFEDS |
| | | | DEGSNWNWNTLLRPNFLDGW |
| | | | YQTLQTHMKKVREQMAGILS |
| | | | RIPEQGVVNWNKIPEGANTT |
| | | | STTKIIDGHVVTINETTYTD |
| | | | GSDDYSTLIRVRVIDVRPQN |
| | | | ETILTTVSSEADSDVTTLPT |
| | | | LIGKNETSTQSSRSVESVED |
| | | | FDNEIPKNQGDVLTA |
| | Ves v 1 | 14 | GPKCPFNSDTVSIIIETREN |
| | | | RNRDLYTLQTLQNHPEFKKK |
| | | | TITRPVVFITHGFTSSASET |
| | | | NFINLAKALVDKDNYMVISI |
| | | | DWQTAACTNEAAGLKYLYYP |
| | | | TAARNTRLVGQYIATITQKL |
| | | | VKHYKISMANIRLIGHS(G) |
| | | | LGAHASGFAGKKVQELKLGK |
| | | | YSEIIGLD(A)PARPSFDSN |
| | | | HCSERLCETDAEYVQIIHTS |
| | | | NYLGTEKTLGTVDFYMNNGK |
| | | | NQPGCGRFFSEVCSHSRAVI |
| | | | YMAECIKHECCLIGIPKSKS |
| | | | SQPISSCTKQECVCVGLNAK |
| | | | KYPSRGSFYVPVESTAPFCN |
| | | | NKGKII |
| | Ves v 5 | 15 | NNYCKIKCLKGGVHTACKYG |
| | | | SLKPNCGNKVVVSYGLTKQE |
| | | | KQDILKEHNDFRQKIARGLE |
| | | | TRGNPGPQPPAKNMKNLVWN |
| | | | DELAYVAQVWANQCQYGHDT |
| | | | CRDVAKYQVGQNVALTGSTA |
| | | | AKYDDPVKLVKMWEDEVKDY |
| | | | NPKKKFSGNDFLKTGHYTQM |
| | | | VWANTKEVGCGSIKYIQEKW |
| | | | HKHYLVCNYGPSGNFMNEEL |
| | | | YQTK |
| Aeroallergen | Lol p 1 | 16 | IAKVPPGPNITAEYGDKWLD |
| | | | AKSTWYGKPTGAGPKDNGGA |
| | | | CGYKNVDKAPFNGMTGCGNT |
| | | | PIFKDGRGCGSCFEIKCTKP |
| | | | ESCSGEAVTVTITDDNEEPI |
| | | | APYH(V)FDLSGHAFGSMAK |
| | | | KGEEQNVRSAGELELQFRRV |
| | | | KCKYPDDTKPTFHVEKASNP |
| | | | NYLAILVKYVDGDGDVVAVD |
| | | | IKEKGKDKWTELKESWGAVW |
| | | | RIDTPDKLTGPFTVRYTTEG |
| | | | GTKSEFEDVIPEGWKADTSY |
| | | | SAK |
| | Lol p 5 | 17 | ADAGYTPAAAATPATPAATP |
| | | | AAAGGKATTDEQKLLEDVNA |
| | | | GFKAAVAAAANAPPADKFKI |
| | | | FEAAFSESSKGLLATSAAKA |
| | | | PGLIPKLDTAYDVAYKAAEA |
| | | | TPEAKYDAFVTALTEALRVI |
| | | | AGALEVHAVKPATEEVLAAK |
| | | | IPTGELQIVDKIDAAFKIAA |
| | | | TAANAAPTNDKFTVFESAFN |
| | | | KALNECTGGAYETYKFIPSL |
| | | | EAAVKQAYAATVAAAPEVKY |
| | | | AVFEAALTKAITAMTQAQKA |
| | | | GKPAAAAATAAATVATAAAT |
| | | | AAAVLPPPLLVVQSLISLLI |
| | | | YY |
| | Phl p 1 | 18 | IPKVPPGPNITATYGDKWLD |
| | | | AKSTWYGKPTAAGPKDNGGA |
| | | | CGYKDVDKPPFSGMTGCGNT |
| | | | PIFKSGRGCGSCFEIKCTKP |
| | | | EACSGEPVVVHITDDNEEPI |
| | | | AAYH(V)FDLSGIAFGSMAK |
| | | | KGDEQKLRSAGEVEIQFRRV |
| | | | KCKYPEGTKVTFHVEKGSNP |
| | | | NYLALLVKFVAGDGDWVAVD |
| | | | IKEKGKDKWIALKESWGAIW |
| | | | RIDTPEVLKGPFTVRYTTEG |
| | | | GTKGEAKDVIPEGWKADTAY |
| | | | ESK |
| | Phl p 5 | 19 | ADLGYGPATPAAPAAGYTPA |
| | | | TPAAPAEAAPAGKATTEEQK |
| | | | LIEKINAGFKAALAAAAGVQ |
| | | | PADKYRTFVATFGAASNKAF |
| | | | AEGLSGEPKGAAESSSKAAL |
| | | | TSKLDAAYKLAYKTAEGATP |
| | | | EAKYDAYVATLSEALRIIAG |
| | | | TLEVHAVKPAAEEVKVIPAG |
| | | | ELQVIEKVDAAFKVAATAAN |
| | | | AAPANDKFTVFEAAFNDAIK |
| | | | ASTGGAYESYKFIPALEAAV |
| | | | KQAYAATVATAPEVKYTVFE |
| | | | TALKKAITAMSEAQKAAKPA |
| | | | AAATATATAAVGAATGAATA |
| | | | ATGGYKV |
| | Bet v 1 | 20 | MGVFNYETETTSVIPAARLF |
| | | | KAFILDGDNLFPKVAPQAIS |
| | | | SVENIEGNGGPGTIKKISFP |
| | | | EGFPFKYVKDRVDEVDHTNF |
| | | | KYNYSVIEGGPIGDTLEKIS |
| | | | NEIKIVATPDGGSILKISNK |
| | | | YHTKGDHEVKAEQVKASKEM |
| | | | GETLLRAVESYLLAHSDAYN |
| | Cyn d 1 | 21 | AMGDKPGPNITATYGDKWLD |
| | | | AKATFYGSDPRGAAPDDHGG |
| | | | ACGYKDVDKAPFDGMTGCGN |
| | | | EPIFKDGLACGSCYEIKCKE |
| | | | PAECSGEPVLIKITDKNYEH |
| | | | IAAYH(V)FDLSGKAFGAMA |
| | | | KKGEEDKLRKAGELMLQFRR |
| | | | VKCEYPSDTKIAFHVEKGSS |
| | | | PNYLALLVKYAAGDGNIVGV |
| | | | DIKPKGSDEFLPMKQSWGAI |
| | | | WRIDPPKPLKGPFTIRLTSE |
| | | | SGGHVEQDDVIPEDWKPDTV |
| | | | YKSKIQF |
| | Pas n 1 | 22 | GPPKVPPGPNITTNYNGKWL |
| | | | PAKATWYGQPNGAGPDDNGG |
| | | | ACGIKNVNLPPYNGFTACGN |
| | | | PPIFKDGKGCGSCYEIRCNK |
| | | | PECSGQPVTVFITDMNYEPI |
| | | | APYH(V)FDLSGKAFGAMAK |
| | | | PGLNDKLRHYGIFDLEFRRV |
| | | | RCKYQGGQKIVFHVEKGSNP |
| | | | NYLAMLVKFVADDGDIVLME |
| | | | LKEKSSDWKPMKLSWGAIWR |
| | | | MDTPKALVPPFSIRLTSESG |
| | | | KKVIAQDVIPVNWKPDTVYN |
| | | | SNVQF |
| | Amb n 1 | 23 | AEDLQEILPVNETRRLTTSG |
| | | | AYNIIDGCWRGKADWAENRK |
| | | | ALADCAQGFGKGTVGGKDGD |
| | | | IYTVTSELDDDVANPKEGTL |
| | | | RFGAAQNRPLWIIFERDMVI |
| | | | RLDKEMVVNSDKTIDGRGAK |
| | | | VEIINAGFTLNGVKNVIIHN |
| | | | INMHDVKVNPGGLIKSNDGP |
| | | | AAPRAGSDGDAISISGSSQI |
| | | | WIDHCSLSKSVDGLVDAKLG |
| | | | TTRLTVSNSLFTQHQFVLLF |
| | | | GAGDENIEDRGMLATVAFNT |
| | | | FTDNVDQRMPRCRHGFFQVV |
| | | | NNNYDKWGSYAIGGSASPTI |
| | | | LSQGNRFCAPDERSKKNVLG |
| | | | RHGEAAAESMKWNWRTNKDV |
| | | | LENGAIFVASGVDPVLTPEQ |
| | | | SAGMIPAEPGESALSLTSSA |
| | | | GVLSCQPGAPC |
| | Parj 1 | 24 | MVRALMPCLPFVQGKEKEPS |
| | | | KGCCSGAKRLDGETKTGPQR |
| | | | VHACECIQTAMKTYSDIDGK |
| | | | LVSEVPKHCGIVDSKLPPID |
| | | | VNMDCKTVGVVPRQPQLPVS |
| | | | LRHGPVTGPSDPAHKARLER |
| | | | PQIRVPPPAPEKA |
| | Par j 2 | 25 | EEACGKVVQDIMPCLHFVKG |
| | | | EEKEPSKECCSGTKKLSEEV |
| | | | KTTEQKREACKCIVRATKGI |
| | | | SGIKNELVAEVPKKCDIKTT |
| | | | LPPITADFDCSKIQSTIFRG |
| | | | YY |
| | Der p 1 | 26 | SSIKTFEEYKKAFNKSYATF |
| | | | EDEEAARKNFLESVKYVQSN |
| | | | GGAINHLSDLSLDEFKNRFL |
| | | | MSAEAFEHLKTQFDLNAETN |
| | | | ACSINGNAPAEIDLRQMRTV |
| | | | TPIRMQGGCGSC(S)WAFSG |
| | | | VAATESAYLAYRNQSLDLAE |
| | | | QELVDCASQHGCHGDTIPRG |
| | | | IEYIQHNGVVQESYYRYVAR |
| | | | EQSCRRPNAQRFGISNYCQI |
| | | | YPPNVNKIREALAQTHSAIA |
| | | | VIIGIKDLDAFRHYDGRTII |
| | | | QRDNGYQPNYHAVNIVGYSN |
| | | | AQGVDYWIVRNSWDTNWGDN |
| | | | GYGYFAANIDLMMIEEYPYV |
| | | | VIL |
| | Der p 2 | 27 | DQVDVKDCANHEIKKVLVPG |
| | | | CHGSEPCIIHRGKPFQLEAV |
| | | | FEANQNTKTAKIEIKASIDG |
| | | | LEVDVPGIDPNACHYMKCPL |
| | | | VKGQQYDIKYTWNVPKIAPK |
| | | | SENVVVTVKVMGDDGVLACA |
| | | | IATHAKIRD |
| | Fel d 1 | 28 | VKMAETCPIFYDVFFAVANG |
| | | | NELLLDLSLTKVNATEPERT |
| | | | AMKKIQDCYVENGLISRVLD |
| | | | GLVMTTISSSKDCMGEAVQN |
| | | | TVEDLKLNTLGREICPAVKR |
| | | | DVDLFLTGTPDEYVEQVAQY |
| | | | KALPVVLENARILKNCVDAK |
| | | | MTEEDKENALSVLDKIYTSP |
| | | | LC |
| | Can f 1 | 29 | QDTPALGKDTVAVSGKWYLK |
| | | | AMTADQEVPEKPDSVTPMIL |
| | | | KAQKGGNLEAKITMLTNGQC |
| | | | QNITVVLHKTSEPGKYTAYE |
| | | | GQRVVFIQPSPVRDHYILYC |
| | | | EGELHGRQIRMAKLLGRDPE |
| | | | QSQEALEDFREFSRAKGLNQ |
| | | | EILELAQSETCSPGGQ |
| Food allergen | Gal d 1 | 30 | AEVDCSRFPNATDKEGKDVL |
| | | | VCNKDLRPICGTDGVTYTND |
| | | | CLLCAYSIEFGTNISKEHDG |
| | | | ECKETVPMNCSSYANTTSED |
| | | | GKVMVLCNRAFNPVCGTDGV |
| | | | TYDNECLLCAHKVEQGASVD |
| | | | KRHDGGCRKELAAVSVDCSE |
| | | | YPKPDCTAEDRPLCGSDNKT |
| | | | YGNKCNFCNAVVESNGTLTL |
| | | | SHFGKC |
| | Gal d 2 | 31 | GSIGAASMEFCFDVFKELKV |
| | | | HHANENIFYCPIAIMSALAM |
| | | | VYLGAKDSTRTQINKVVRFD |
| | | | KLPGFGDSIEAQCGTSVNVH |
| | | | SSLRDILNQITKPNDVYSFS |
| | | | LASRLYAEERYPILPEYLQC |
| | | | VKELYRGGLEPINFQTAADQ |
| | | | ARELINSWVESQTNGIIRNV |
| | | | LQPSSVDSQTAMVLVNAIVF |
| | | | KGLWEKAFKDEDTQAMPFRV |
| | | | TEQESKPVQMMYQIGLFRVA |
| | | | SMASEKMKILELPFASGTMS |
| | | | MLVLLPDEVSGLEQLESIIN |
| | | | FEKLTEWTSSNVMEERKIKV |
| | | | YLPRMKMEEKYNLTSVLMAM |
| | | | GITDVFSSSANLSGISSAES |
| | | | LKISQAVHAAHAEINEAGRE |
| | | | VVGSAEAGVDAASVSEEFRA |
| | | | DHPFLFCIKHIATNAVLFFG |
| | | | RCVSP |
| | Bos d 4 | 32 | EQLTKCEVFRELKDLKGYGG |
| | | | VSLPEWVCTTFHTSGYDTQA |
| | | | IVQNNDSTEYGLFQINNKIW |
| | | | CKDDQNPHSSNICNISCDKF |
| | | | LDDDLTDDIMCVKKILDKVG |
| | | | INYWLAHKALCSEKLDQWLC |
| | | | EKL |
| | Bos d 5 | 33 | LIVTQTMKGLDIQKVAGTWY |
| | | | SLAMAASDISLLDAQSAPLR |
| | | | VYVEELKPTPEGDLEILLQK |
| | | | WENGECAQKKIIAEKTKIPA |
| | | | VFKIDALNENKVLVLDTDYK |
| | | | KYLLFCMENSAEPEQSLACQ |
| | | | CLVRTPEVDDEALEKFDKAL |
| | | | KALPMHIRLSFNPTQLEEQC |
| | | | HI |
| | Ara h 1 | 34 | KSSPYQKKTENPCAQRCLQS |
| | | | CQQEPDDLKQKACESRCTKL |
| | | | EYDPRCVYDPRGHTGTTNQR |
| | | | SPPGERTRGRQPGDYDDDRR |
| | | | QPRREEGGRWGPAGPRERER |
| | | | EEDWRQPREDWRRPSHQQPR |
| | | | KIRPEGREGEQEWGTPGSHV |
| | | | REETSRNNPFYFPSRRFSTR |
| | | | YGNQNGRIRVLQRFDQRSRQ |
| | | | FQNLQNHRIVQIEAKPNTLV |
| | | | LPKHADADNILVIQQGQATV |
| | | | TVANGNNRKSFNLDEGHALR |
| | | | IPSGFISYILNRHDNQNLRV |
| | | | AKISMPVNTPGQFEDFFPAS |
| | | | SRDQSSYLQGFSRNTLEAAF |
| | | | NAEFNEIRRVLLEENAGGEQ |
| | | | EERGQRRWSTRSSENNEGVI |
| | | | VKVSKEHVEELTKHAKSVSK |
| | | | KGSEEEGDITNPINLREGEP |
| | | | DLSNNFGKLFEVKPDKKNPQ |
| | | | LQDLDMMLTCVEIKEGALML |
| | | | PHFNSKAMVIVVVNKGTGNL |
| | | | ELVAVRKEQQQRGRREEEED |
| | | | EDEEEEGSNREVRRYTARLK |
| | | | EGDVFIMPAAHPVAINASSE |
| | | | LHLLGFGINAENNHRIFLAG |
| | | | DKDNVIDQIEKQAKDLAFPG |
| | | | SGEQVEKLIKNQKESHFVSA |
| | | | RPQSQSQSPSSPEKESPEKE |
| | | | DQEEENQGGKGPLLSILKAF |
| | | | N |
| | Ara h 2 | 35 | RQQWELQGDRRCQSQLERAN |
| | | | LRPCEQHLMQKIQRDEDSYG |
| | | | RDPYSPSQDPYSPSQDPDRR |
| | | | DPYSPSPYDRRGAGSSQHQE |
| | | | RCCNELNEFENNQRCMCEAL |
| | | | QQIMENQSDRLQGRQQEQQF |
| | | | KRELRNLPQQCGLRAPQRCD |
| | | | LEVESGGRDRY |
| | Ara h 3 | 36 | ISFRQQPEENACQFQRLNAQ |
| | | | RPDNRIESEGGYIETWNPNN |
| | | | QEFECAGVALSRLVLRRNAL |
| | | | RRPFYSNAPQEIFIQQGRGY |
| | | | FGLIFPGCPSTYEEPAQQGR |
| | | | RYQSQRPPRRLQEEDQSQQQ |
| | | | QDSHQKVHRFNEGDLIAVPT |
| | | | GVAFWLYNDHDTDVVAVSLT |
| | | | DTNNNDNQLDQFPRRFNLAG |
| | | | NHEQEFLRYQQQSRQSRRRS |
| | | | LPYSPYSPHSRPRREEREFR |
| | | | PRGQHSRRERAGQEEEDEGG |
| | | | NIFSGFTPEFLEQAFQVDDR |
| | | | QIVQNLWGENESEEEGAIVT |
| | | | VRGGLRILSPDGTRGADEEE |
| | | | EYDEDQYEYHEQDGRRGRGS |
| | | | RGGGNGIEETICTACVKKNI |
| | | | GGNRSPHIYDPQRWFTQNCH |
| | | | DLNLLILRWLGLSAEYGNLY |
| | | | RNALFVPHYNTNAHSIIYAL |
| | | | RGRAHVQVVDSNGNRVYDEE |
| | | | LQEGHVLVVPQNFAVAGKSQ |
| | | | SENFEYVAFKTDSRPSIANF |
| | | | AGENSFIDNLPEEVVANSYG |
| | | | LPREQARQLKNNNPFKFFVP |
| | | | PFQQSPRAVA |
| | Ara h 6 | 37 | MRRERGRQGDSSSCERQVDG |
| | | | VNLKPCEQHIMQRIMGEQEQ |
| | | | YDSYNFGSTRSSDQQQRCCD |
| | | | ELNEMENTQRCMCEALQQIM |
| | | | ENQCDGLQDRQMVQHFKREL |
| | | | MNLPQQCNFGAPQRCDLDVS |
| | | | GGRC |
| | Ara h 8 | 38 | MGVFTFEDEITSTVPPAKLY |
| | | | NAMKDADSITPKIIDDVKSV |
| | | | EIVEGNGGPGTIKKLTIVED |
| | | | GETKFILHKVESIDEANYAY |
| | | | NYSVVGGVALPPTAEKITFE |
| | | | TKLVEGPNGGSIGKLTLKYH |
| | | | TKGDAKPDEEELKKGKAKGE |
| | | | GLFRAIEGYVLANPTQY |
| | Cor a 9 | 39 | INVGLRRQQQRYFGECNLDR |
| | | | LNALEPTNRIEAEACQIESW |
| | | | DHNDQQFQCAGVAVIRRTIE |
| | | | PNGLLLPQYSNAPELIYIER |
| | | | GRGITGVLFPGCPETFEDPQ |
| | | | QQSQQGQRQGQGQSQRSEQD |
| | | | RHQKIRHFREGDIIALPAGV |
| | | | AHWCYNDGDSPVVTVSLLHT |
| | | | NNYANQLDENPRHFYLAGNP |
| | | | DDEHQRQGQQQFGQRRRQQQ |
| | | | HSHGEQGEQEQQGEGNNVFS |
| | | | GFDAEFLADAFNVDVDTARR |
| | | | LQSNQDKRRNIVKVEGRLQW |
| | | | VRPERSRQEWERQERQERES |
| | | | EQERERQRRQGGRGRDVNGF |
| | | | EETICSLRLRENICTRSRAD |
| | | | IYTEQVGRINTVNSNTLPVL |
| | | | RWLQLSAERGDLQREGLYVP |
| | | | HWNLNAHSVVYAIRGRARVQ |
| | | | VVDDNGNTVFDDELRQGQVL |
| | | | TIPQNFAVAKRAESEGFEWV |
| | | | AFKTNDNAQISPLAGRTSAI |
| | | | RALPDDVLANAFQISREEAR |
| | | | RLKYNRQETTLVRSSRSSSE |
| | | | RKRRSESEGRAEA |
| | Cor a 14 | 40 | FRTTITTVDVDEDIVNQQGR |
| | | | RGESCREQAQRQQNLNQCQR |
| | | | YMRQQSQYGSYDGSNQQQQQ |
| | | | ELEQCCQQLRQMDERCRCEG |
| | | | LRQAVMQQQGEMRGEEMREV |
| | | | METARDLPNQCRLSPQRCEI |
| | | | RSARF |
| | Ana o 3 | 41 | SIYRAIVEVEEDSGREQSCQ |
| | | | RQFEEQQRFRNCQRYVKQEV |
| | | | QRGGRYNQRQESLRECCQEL |
| | | | QEVDRRCRCQNLEQMVRQLQ |
| | | | QQEQIKGEEVRELYETASEL |
| | | | PRICSISPSQGCQFQSSY |
| | Jug r 1 | 42 | FRTTITTMEIDEDIDNPRRR |
| | | | GEGCREQIQRQQNLNHCQYY |
| | | | LRQQSRSGGYDEDNQRQHFR |
| | | | QCCQQLSQMDEQCQCEGLRQ |
| | | | VVRRQQQQQGLRGEEMEEMV |
| | | | QSARDLPNECGISSQRCEIR |
| | | | RSWF |
| | Ber e 1 | 43 | FRATVTTTVVEEENQEECRE |
| | | | QMQRQQMLSHCRMYMRQQME |
| | | | ESPYQTMPRRGMEPHMSECC |
| | | | EQLEGMDESCRCEGLRMMMM |
| | | | RMQQEEMQPRGEQMRRMMRL |
| | | | AENIPSRCNLSPMRCPMGGS |
| | | | IAGF |
| | Ses i 1 | 44 | TTYTTTVTTTAIDDEANQQS |
| | | | QQCRQQLQGRQFRSCQRYLS |
| | | | QGRSPYGGEEDEVLEMSTGN |
| | | | QQSEQSLRDCCQQLRNVDER |
| | | | CRCEAIRQAVRQQQQEGGYQ |
| | | | EGQSQQVYQRARDLPRRCNM |
| | | | RPQQCQFRVIFV |
| | Fag e 3 | 45 | GDYPFEPRRQIEHCRQRCQD |
| | | | RSQGFRETRECLETCEKRAG |
| | | | FDDEPSKEVDSYTTDTRSRD |
| | | | PQQEFRQCRHRCQTQEEGGR |
| | | | QQRKCEQRCEKQLERQQGYD |
| | | | DEEFGQGRSNIGAPVRNYDD |
| | | | CTEMCGGSPLCALFCE |
| | Pru du 6 | 46 | ARQSQLSPQNQCQLNQLQAR |
| | | | EPDNRIQAEAGQIETWNFNQ |
| | | | EDFQCAGVAASRITIQRNGL |
| | | | HLPSYSNAPQLIYIVQGRGV |
| | | | LGAVFSGCPETFEESQQSSQ |
| | | | QGRQQEQEQERQQQQQGEQG |
| | | | RQQGQQEQQQERQGRQQGRQ |
| | | | QQEEGRQQEQQQGQQGRPQQ |
| | | | QQQFRQFDRHQKTRRIREGD |
| | | | VVAIPAGVAYWSYNDGDQEL |
| | | | VAVNLFHVSSDHNQLDQNPR |
| | | | KFYLAGNPENEFNQQGQSQP |
| | | | RQQGEQGRPGQHQQPFGRPR |
| | | | QQEQQGSGNNVFSGENTQLL |
| | | | AQALNVNEETARNLQGQNDN |
| | | | RNQIIRVRGNLDFVQPPRGR |
| | | | QEREHEERQQEQLQQERQQQ |
| | | | GGQLMANGLEETFCSLRLKE |
| | | | NIGNPERADIFSPRAGRIST |
| | | | LNSHNLPILRFLRLSAERGF |
| | | | FYRNGIYSPHWNVNAHSVVY |
| | | | VIRGNARVQVVNENGDAILD |
| | | | QEVQQGOLFIVPQNHGVIQQ |
| | | | AGNQGFEYFAFKTEENAFIN |
| | | | TLAGRTSFLRALPDEVLANA |
| | | | YQISREQARQLKYNRQETIA |
| | | | LSSSQQRRAVV |
| Latex | Hev b 5 | 47 | ASVEVESAATALPKNETPEV |
| allergen | | | TKAEETKTEEPAAPPASEQE |
| | | | TADATPEKEEPTAAPAEPEA |
| | | | PAPETEKAEEVEKIEKTEEP |
| | | | APEADQTTPEEKPAEPEPVA |
| | | | EEEPKHETKETETEAPAAPA |
| | | | EGEKPAEEEKPITEAAETAT |
| | | | TEVPVEKTEE |
| | Hev b 6 | 48 | EQCGRQAGGKLCPNNLCCSQ |
| | | | WGWCGSTDEYCSPDHNCQSN |
| | | | CKDSGEGVGGGSASNVLATY |
| | | | HLYNSQDHGWDLNAASAYCS |
| | | | TWDANKPYSWRSKYGWTAFC |
| | | | GPVGAHGQSSCGKCLSVTNT |
| | | | GTGAKTTVRIVDQCSNGGLD |
| | | | LDVNVFRQLDTDGKGYERGH |
| | | | ITVNYQFVDCGDSFNPLFSV |
| | | | MKSSVIN |
| Mold | Alt a 1 | 49 | APLESRQDTASCPVTTEGDY |
| allergen | | | VWKISEFYGRKPEGTYYNSL |
| | | | GFNIKATNGGTLDFTCSAQA |
| | | | DKLEDHKWYSCGENSFMDFS |
| | | | FDSDRSGLLLKQKVSDDITY |
| | | | VATATLPNYCRAGGNGPKDF |
| | | | VCQGVADAYITLVTLPKSS |
| | Asp f 1 | 50 | APSPLDARATWTCINQQLNP |
| | | | KTNKWEDKRLLYNQAKAESN |
| | | | SHHAPLSDGKTGSSYPHWFT |
| | | | NGYDGNGKLIKGRTPIKFGK |
| | | | ADCDRPPKHSQNGMGKDDHY |
| | | | LLEFPTFPDGHDYKFDSKKP |
| | | | KEDPGPARVIYTYPNKVFCG |
| | | | IVAHQRGNQGDLRLCSH |
| | Cla h 8 | 51 | MPGQQATKHESLLDQLSLKG |
| | | | KVVVVTGASGPKGMGIEAAR |
| | | | GCAEMGAAVAITYASRAQGA |
| | | | EENVKELEKTYGIKAKAYKC |
| | | | QVDSYESCEKLVKDVVADFG |
| | | | QIDAFIANAGATADSGILDG |
| | | | SVEAWNHVVQVDLNGTFHCA |
| | | | KAVGHHFKERGTGSLVITAS |
| | | | MSGHIANFPQEQTSY(F)NV |
| | | | AK(A)AGCIHMARSLANEWR |
| | | | DFARVNSISPGYIDTGLSDF |
| | | | VPKETQQLWHSMIPMGRDGL |
| | | | AKELKGAYVYFASDASTYTT |
| | | | GADLLIDGGYTTR |
| Insect venom | Api m 1 | 52 | ATGCAAGTCGTTCTCGGATC |
| allergen | | | CTTGTTCCTTCTCCTCCTCT |
| | | | CTACCTCTCACGGATGGCAA |
| | | | ATCAGGGATAGGATCGGGGA |
| | | | TAACGAGTTGGAGGAACGGA |
| | | | TAATATATCCAGGAACGTTA |
| | | | TGGTGCGGGCATGGTAACAA |
| | | | GTCGTCCGGCCCGAACGAGC |
| | | | TAGGTCGGTTCAAGCACACG |
| | | | GATGCATGCTGTCGAACCCA |
| | | | GGACATGTGCCCGGACGTGA |
| | | | TGTCAGCTGGTGAATCGAAG |
| | | | CACGGCCTGACCAACACGGC |
| | | | CTCCCACACCAGGTTGTCGT |
| | | | GCGACTGCGACGACAAGTTC |
| | | | TAT |
| | | | GATTGTCTTAAAAATTCGGC |
| | | | GGACACGATTAGCTCGTATT |
| | | | TCGTAGGGAAGATGTACTTC |
| | | | AATCTGATAGACACGAAGTG |
| | | | TTACAAACTGGAGCATCCTG |
| | | | TCACCGGGTGCGGTGAGAGA |
| | | | ACCGAGGGTCGTTGTCTTCA |
| | | | CTACACCGTGGACAAAAGCA |
| | | | AACCGAAAGTGTACCAATGG |
| | | | TTCGATCTTCGCAAGTATGG |
| | | | AGGCGGAGGCAGCCACCACC |
| | | | ATCACCATCATCCAGGAGGA |
| | | | GGCCTGAACGACATCTTCGA |
| | | | GGCTCAGAAGATCGAGTGGC |
| | | | ACGAGTGA |
| Aero | Lol p 1 | 53 | ATGCAGGTCGTGCTGGGTTC |
| allergen | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAACGTA |
| | | | TCGCTAAGGTGCCACCTGGT |
| | | | CCTAACATCACCGCTGAGTA |
| | | | CGGCGACAAGTGGCTGGACG |
| | | | CTAAGTCCACTTGGTACGGC |
| | | | AAGCCTACCGGTGCTGGTCC |
| | | | CAAGGATAACGGTGGTGCTT |
| | | | GCGGTTACAAGAACGTGGAC |
| | | | AAGGCTCCCTTCAACGGCAT |
| | | | GACCGGTTGCGGCAACACCC |
| | | | CTATCTTCAAGGACGGTCGT |
| | | | GGTTGCGGCTCCTGCTTCGA |
| | | | GATCAAGTGCACCAAGCCTG |
| | | | AGTCCTGCTCTGGCGAGGCT |
| | | | GTGACCGTGACTATCACCGA |
| | | | CGACAACGAAGAACCGATCG |
| | | | CTCCCTACGTGTTCGACCTG |
| | | | TCCGGTCACGCTTTCGGTTC |
| | | | CATGGCTAAGAAGGGCGAAG |
| | | | AACAGAACGTGCGTTCCGCT |
| | | | GGCGAACTCGAGCTGCAATT |
| | | | CCGTCGTGTGAAGTGCAAGT |
| | | | ACCCCGACGACACCAAGCCA |
| | | | ACCTTCCACGTCGAGAAGGC |
| | | | TTCTAACCCCAACTACCTGG |
| | | | CTATCCTGGTCAAATACGTG |
| | | | GACGGCGA |
| | | | CGGCGACGTGGTGGCTGTGG |
| | | | ACATCAAAGAGAAGGGCAAA |
| | | | GACAAGTGGACCGAGCTGAA |
| | | | AGAATCCTGGGGTGCTGTGT |
| | | | GGCGTATCGACACCCCTGAC |
| | | | AAGCTGACCGGTCCTTTCAC |
| | | | CGTGCGTTACACCACCGAAG |
| | | | GTGGTACTAAGTCCGAGTTC |
| | | | GAGGACGTGATCCCCGAAGG |
| | | | CTGGAAGGCTGACACCTCCT |
| | | | ACTCCGCTAAAGGTGGTGGT |
| | | | GGTTCCCACCATCACCATCA |
| | | | TCACCCTGGTGGTGGCCTGA |
| | | | ACGACATCTTCGAGGCTCAA |
| | | | AAGATCGAGTGGCACGAATA |
| | | | A |
| Vaccine | HA_A- | 54 | ATGAAGGCCATCCTGGTGGT |
| antigen | Michigan- | | GCTGCTGTACACCTTCACCA |
| | 2015 | | CCGCCAACGCCGACACACTG |
| | | | TGTATCGGCTACCACGCCAA |
| | | | CAACAGCACCGACACCGTGG |
| | | | ATACCGTGCTGGAAAAGAAC |
| | | | GTGACCGTGACACACAGCGT |
| | | | GAACCTGCTGGAAGATAAGC |
| | | | ACAACGGCAAGCTGTGCAAG |
| | | | CTGAGAGGCGTGGCACCTCT |
| | | | GCACCTGGGCAAGTGTAATA |
| | | | TCGCCGGCTGGATTCTGGGC |
| | | | AACCCTGAGTGTGAAAGCCT |
| | | | GAGCACAGCCAGCAGCTGGT |
| | | | CCTACATCGTGGAAACCAGC |
| | | | AACAGCGACAACGGCACATG |
| | | | CTTCCCCGGCGACTTCATCA |
| | | | ACTACGAGGAACTGCGGGAA |
| | | | CAGCTGAGCAGCGTGTCCAG |
| | | | CTTCGAGAGATTCGAGATCT |
| | | | TCCCCAAGACCTCCAGCTGG |
| | | | CCCAACCACGATTCTAACAA |
| | | | GGGCGTGACAGCCGCCTGTC |
| | | | CTCATGCCGGCGCTAAGAGC |
| | | | TTCTACAAGAACCTGATCTG |
| | | | GCTGGTCAAGAAGGGCAACA |
| | | | GCTACCCCAAGCTGAACCAG |
| | | | AGCTACATCAACGACAAGGG |
| | | | CAAAGAGGTGCTGGTCCTCT |
| | | | GGGGCATCCACCATCCTAGC |
| | | | ACAACAGCCGATCAGCAGAG |
| | | | CCTGTACCAGAACGCCGATG |
| | | | CCTATGTGTTCGTGGGCACC |
| | | | AGCCGGTACAGCAAGAAGTT |
| | | | CAAGCCCGAGATCGCCACCA |
| | | | GACCTAAAGTGCGGGACAGA |
| | | | GAAGGCCGGATGAACTACTA |
| | | | CTGGACCCTGGTGGAACCCG |
| | | | GCGACAAGATCACATTTGAG |
| | | | GCCACCGGCAACCTGGTGGT |
| | | | CCCCAGATACGCCTTCACCA |
| | | | TGGAAAGAAATGCCGGCAGC |
| | | | GGCATCATCATCAGCGACAC |
| | | | CCCTGTGCACGACTGCAACA |
| | | | CCACCTGTCAGACACCTGAG |
| | | | GGCGCCATCAATACCAGCCT |
| | | | GCCTTTCCAGAACATTCACC |
| | | | CCATCACCATCGGCAAGTGC |
| | | | CCCAAATACGTGAAGTCCAC |
| | | | CAAGCTGAGGCTGGCCACAG |
| | | | GCCTGAGAAATGTGCCCTCC |
| | | | ATCCAGAGCAGAGGCCTGTT |
| | | | TGGAGCCATTGCCGGCTTTA |
| | | | TCGAAGGCGGCTGGACAGGC |
| | | | ATGGTGGACGGATGGTACGG |
| | | | ATACCACCACCAGAACGAGC |
| | | | AAGGCTCTGGCTATGCCGCC |
| | | | GACCTGAAGTCTACCCAGAA |
| | | | TGCCATCGATAAGATCACCA |
| | | | ACAAAGTGAACAGCGTGATC |
| | | | GAGAAGATGAACACCCAGTT |
| | | | CACCGCCGTGGGAAAAGAGT |
| | | | TCAACCACCTGGAAAAGCGC |
| | | | ATCGAGAACCTGAACAAGAA |
| | | | GGTGGACGACGGCTTCCTGG |
| | | | ACATCTGGACCTACAATGCC |
| | | | GAGCTGCTGGTGCTCCTGGA |
| | | | AAACGAGAGAACCCTGGACT |
| | | | ACCACGACAGCAACGTGAAG |
| | | | AACCTGTACGAGAAAGTGCG |
| | | | CAACCAGCTGAAGAACAACG |
| | | | CCAAAGAGATCGGCAACGGC |
| | | | TGCTTCGAGTTCTACCACAA |
| | | | GTGCGACAATACCTGCATGG |
| | | | AAAGCGTGAAGAATGGCACC |
| | | | TACGACTACCCTAAGTACAG |
| | | | CGAGGAAGCCAAACTGAACC |
| | | | GCGAGAAGATCGACGGCTCC |
| | | | GGATATATCCCTGAGGCTCC |
| | | | TAGAGATGGCCAGGCCTACG |
| | | | TCAGAAAGGATGGCGAGTGG |
| | | | GTCCTGCTGAGCACCTTTCT |
| | | | CGGATCCGGCCTGAACGACA |
| | | | TCTTCGAGGCCCAGAAAATC |
| | | | GAGTGGCACGAGGGCCACCA |
| | | | CCATCATCACCATTGA |
| | HA_B- | 55 | ATGAAGGCCATCATCGTGCT |
| | Phuket- | | GCTGATGGTGGTCACCAGCA |
| | 2013 | | ACGCCGACAGAATCTGCACC |
| | | | GGCATCACCAGCAGCAACAG |
| | | | CCCTCACGTGGTCAAGACAG |
| | | | CCACACAGGGCGAAGTGAAT |
| | | | GTGACCGGCGTGATCCCTCT |
| | | | GACCACCACACCTACCAAGA |
| | | | GCTACTTCGCCAACCTGAAG |
| | | | GGCACCAGAACCAGAGGCAA |
| | | | GCTGTGCCCCGATTGCCTGA |
| | | | ACTGCACCGATCTGGATGTG |
| | | | GCCCTGGGCAGACCTATGTG |
| | | | CGTGGGAACAACACCTAGCG |
| | | | CCAAGGCCAGCATCCTGCAT |
| | | | GAAGTGCGGCCTGTGACCAG |
| | | | CGGCTGCTTCCCTATTATGC |
| | | | ACGACCGGACCAAGATCAGA |
| | | | CAGCTGCCCAATCTGCTGCG |
| | | | GGGCTACGAGAAGATCAGGC |
| | | | TGAGCACCCAGAACGTGATC |
| | | | GACGCCGAAAAAGCTCCTGG |
| | | | CGGCCCTTACAGACTGGGCG |
| | | | GATCTGGCTCTTGCCCCAAC |
| | | | GCTACAAGCAAGATCGGCTT |
| | | | CTTCGCCACCATGGCCTGGG |
| | | | CCGTGCCTAAGGACAACTAC |
| | | | AAGAACGCCACCAATCCTCT |
| | | | GACCGTGGAAGTGCCCTACA |
| | | | TCTGTACCGAAGGCGAGGAC |
| | | | CAGATCACCGTGTGGGGCTT |
| | | | CCACAGCGACAACAAGACCC |
| | | | AGATGAAGTCCCTGTACGGC |
| | | | GACAGCAACCCTCAGAAGTT |
| | | | TACCAGCAGCGCCAACGGCG |
| | | | TGACCACACACTATGTGTCC |
| | | | CAGATCGGCGACTTCCCCGA |
| | | | CCAGACAGAAGATGGCGGAC |
| | | | TGCCTCAGAGCGGCAGAATC |
| | | | GTGGTGGACTACATGATGCA |
| | | | GAAGCCCGGCAAGACCGGCA |
| | | | CCATCGTGTATCAGAGAGGC |
| | | | GTCCTGCTGCCACAGAAAGT |
| | | | TTGGTGCGCCAGCGGCCGGT |
| | | | CCAAAGTGATCAAAGGATCT |
| | | | CTGCCTCTGATCGGCGAGGC |
| | | | CGACTGTCTGCACGAAGAAT |
| | | | ATGGCGGCCTGAACAAGAGC |
| | | | AAGCCCTACTACACAGGCAA |
| | | | GCACGCCAAAGCCATCGGCA |
| | | | ACTGCCCTATCTGGGTCAAG |
| | | | ACCCCTCTGAAGCTGGCCAA |
| | | | CGGCACCAAGTATAGACCTC |
| | | | CAGCCAAGCTGCTGAAAGAG |
| | | | CGGGGCTTCTTTGGAGCTAT |
| | | | CGCCGGCTTTCTTGAAGGCG |
| | | | GCTGGGAGGGAATGATTGCC |
| | | | GGCTGGCATGGCTACACATC |
| | | | TCATGGCGCACATGGCGTGG |
| | | | CAGTGGCCGCTGATCTGAAG |
| | | | TCTACACAAGAGGCCATCAA |
| | | | CAAGATCACCAAGAACCTGA |
| | | | ACAGCCTGAGCGAGCTGGAA |
| | | | GTGAAGAACCTGCAGAGACT |
| | | | GTCCGGCGCCATGGACGAGC |
| | | | TGCACAACGAGATCCTGGAA |
| | | | CTGGACGAGAAGGTGGACGA |
| | | | CCTGAGAGCCGATACCATCT |
| | | | CCAGCCAGATTGAGCTGGCA |
| | | | GTGCTGCTGTCCAACGAGGG |
| | | | CATCATCAACAGCGAGGACG |
| | | | AGCATCTGCTGGCCCTGGAA |
| | | | CGGAAGCTGAAGAAGATGCT |
| | | | GGGACCCAGCGCCGTGGATA |
| | | | TCGGCAATGGCTGCTTCGAG |
| | | | ACAAAGCACAAGTGCAACCA |
| | | | GACCTGCCTGGACAGAATTG |
| | | | CCGCCGGAACCTTTAACGCC |
| | | | GGCGAGTTTAGCCTGCCTAC |
| | | | CTTCGACAGCCTGAACATCA |
| | | | CAGGCTCCGGATATATCCCT |
| | | | GAGGCTCCTAGAGATGGCCA |
| | | | GGCCTACGTCAGAAAGGATG |
| | | | GCGAGTGGGTCCTGCTGAGC |
| | | | ACCTTTCTCGGATCCGGCCT |
| | | | GAACGACATCTTCGAGGCCC |
| | | | AGAAAATCGAGTGGCACGAG |
| | | | GGCCACCACCATCATCACCA |
| | | | TTGA |
| | HA_A_Brisb | 56 | MKAILVVLLYTFTTANADTL |
| | ane-2018 | | CIGYHANNSTDTVDTVLEKN |
| | | | VTVTHSVNLLEDKHNGKLCK |
| | | | LGGVAPLHLGKCNIAGWILG |
| | | | NPECESLSTARSWSYIVETS |
| | | | NSDNGTCY(F)PGDFINYEE |
| | | | LREQLSSVSSFERFEIFPKT |
| | | | SSWPNHDSNKGVTAACPHAG |
| | | | AKSFYKNLIWLVKKGNSYPK |
| | | | LNQTYINDKGKEVLVLWGIH |
| | | | HPPTTADQQSLYQNADAYVF |
| | | | VGTSRYSKKFKPEIATRPKV |
| | | | RDREGRMNYYWTLVEPGDKI |
| | | | TFEATGNLVVPRYAFTMERN |
| | | | AGSGIIISDTPVHDCNTTCQ |
| | | | TAEGAINTSLPFQNVHPVTI |
| | | | GKCPKYVKSTKLRLATGLRN |
| | | | VPSIQSRGLFGAIAGFIEGG |
| | | | WTGMVDGWYGYHHQNEQGSG |
| | | | YAADLKSTQNAIDKITNKVN |
| | | | SVIEKMNTQFTAVGKEFNHL |
| | | | EKRIENLNKKVDDGFLDIWT |
| | | | YNAELLVLLENERTLDYHDS |
| | | | NVKNLYEKVRNQLKNNAKEI |
| | | | GNGCFEFYHKCDNTCMESVK |
| | | | NGTYDYPKYSEEAKLNREKI |
| | | | DGSGYIPEAPRDGQAYVRKD |
| | | | GEWVLLSTFLGS |
| | N_SARS- | 57 | MSDNGPQNQRNAPRITFGGP |
| | Cov2 | | SDSTGSNQNGERSGARSKQR |
| | | | RPQGLPNNTASWFTALTQHG |
| | | | KEDLKFPRGQGVPINTNSSP |
| | | | DDQIGYYRRATRRIRGGDGK |
| | | | MKDLSPRWYFYYLGTGPEAG |
| | | | LPYGANKDGIIWVATEGALN |
| | | | TPKDHIGTRNPANNAAIVLQ |
| | | | LPQGTTLPKGFYAEGSRGGS |
| | | | QASSRSSSRSRNSSRNSTPG |
| | | | SSRGTSPARMAGNGGDAALA |
| | | | LLLLDRLNQLESKMSGKGQQ |
| | | | QQGQTVTKKSAAEASKK |
| | | | PRQK(GGPRQG)RTATKAYN |
| | | | VTQAFGRRGPEQTQGNFGDQ |
| | | | ELIRQGTDYKHWPQIAQFAP |
| | | | SASAFFGMSRIGMEVTPSGT |
| | | | WLTYTGAIKLDDKDPNFKDQ |
| | | | VILLNKHIDAYKTFPPTEPK |
| | | | KDKKKKADETQALPQRQKKQ |
| | | | QTVTLLPAADLDDFSKQLQQ |
| | | | SMSSADSTQA |
| | N_SARS- | 58 | MSDNGPQSNQ |
| | Cov | | RSAPRITFGGPTDSTDNNQN |
| | | | GGRNGARPKQRRPQGLPNNT |
| | | | ASWFTALTQHGKEELRFPRG |
| | | | QGVPINTNSGPDDQIGYYRR |
| | | | ATRRVRGGDGKMKELSPRWY |
| | | | FYYLGTGPEASLPYGANKEG |
| | | | IVWVATEGALNTPKDHIGTR |
| | | | NPNNNAATVLQLPQGTTLPK |
| | | | GFYAEGSRGGSQASSRSSSR |
| | | | SRGNSRNSTPGSSRGNSPAR |
| | | | MASGGGETALALLLLDRLNQ |
| | | | LESKVSGKGQQQQGQTVTKK |
| | | | SAAEASKKPRQKRTATKQYN |
| | | | VTQAFGRRGPEQTQGNFGDQ |
| | | | DLIRQGTDYKHWPQIAQFAP |
| | | | SASAFFGMSRIGMEVTPSGT |
| | | | WLTYHGAIKLDDKDPQFKDN |
| | | | VILLNKHIDAYKTFPPTEPK |
| | | | KDKKKKTDEAQPLPQRQKKQ |
| | | | PTVTLLPAADMDDFSRQLQN |
| | | | SMSGASADSTQA |
| | S_SARS- | 59 | MFVFLVLLPLVSSQCVNLTT |
| | Cov2 | | RTQLPPAYTNSFTRGVYYPD |
| | | | KVFRSSVLHSTQDLFLPFFS |
| | | | NVTWFHAIHVSGTNGTKRFD |
| | | | NPVLPFNDGVYFASTEKSNI |
| | | | IRGWIFGTTLDSKTQSLLIV |
| | | | NNATNVVIKVCEFQFCNDPF |
| | | | LGVYYHKNNKSWMESEFRVY |
| | | | SSANNCTFEYVSQPFLMDLE |
| | | | GKQGNFKNLREFVFKNIDGY |
| | | | FKIYSKHTPINLVRDLPQGF |
| | | | SALEPLVDLPIGINITRFQT |
| | | | LLALHRSYLTPGDSSSGWTA |
| | | | GAAAYYVGYLQPRTFLLKYN |
| | | | ENGTITDAVDCALDPLSETK |
| | | | CTLKSFTVEKGIYQTSNFRV |
| | | | QPTESIVRFPNITNLCPFGE |
| | | | VFNATRFASVYAWNRKRISN |
| | | | CVADYSVLYNSASFSTFKCY |
| | | | GVSPTKLNDLCFTNVYADSF |
| | | | VIRGDEVRQIAPGQTGKIAD |
| | | | YNYKLPDDFTGCVIAWNSNN |
| | | | LDSKVGGNYNYLYRLFRKSN |
| | | | LKPFERDISTEIYQAGSTPC |
| | | | NGVEGFNCYFPLQSYGFQPT |
| | | | NGVGYQPYRVVVLSFELLHA |
| | | | PATVCGPKKSTNLVKNKCVN |
| | | | FNFNGLTGTGVLTESNKKFL |
| | | | PFQQFGRDIADTTDAVRDPQ |
| | | | TLEILDITPCSFGGVSVITP |
| | | | GTNTSNQVAVLYQDVNCTEV |
| | | | PVAIHADQLTPTWRVYSTGS |
| | | | NVFQTRAGCLIGAEHVNNSY |
| | | | ECDIPIGAGICASYQTQTNS |
| | | | PRRAR(SGAG)SVASQSIIA |
| | | | YTMSLGAENSVAYSNNSIAI |
| | | | PTNFTISVTTEILPVSMTKT |
| | | | SVDCTMYICGDSTECSNLLL |
| | | | QYGSFCTQLNRALTGIAVEQ |
| | | | DKNTQEVFAQVKQIYKTPPI |
| | | | KDFGGFNFSQILPDPSKPSK |
| | | | RSFIEDLLENKVTLADAGFI |
| | | | KQYGDCLGDIAARDLICAQK |
| | | | FNGLTVLPPLLTDEMIAQYT |
| | | | SALLAGTITSGWTFGAGAAL |
| | | | QIPFAMQMAYRFNGIGVTQN |
| | | | VLYENQKLIANQFNSAIGKI |
| | | | QDSLSSTASALGKLQDVVNQ |
| | | | NAQALNTLVKQLSSNFGAIS |
| | | | SVLNDILSRLDKV(PP)EAE |
| | | | VQIDRLITGRLQSLQTYVTQ |
| | | | QLIRAAEIRASANLAATKMS |
| | | | ECVLGQSKRVDFCGKGYHLM |
| | | | SFPQSAPHGVVFLHVTYVPA |
| | | | QEKNFTTAPAICHDGKAHFP |
| | | | REGVFVSNGTHWFVTQRNFY |
| | | | EPQIITTDNTFVSGNCDVVI |
| | | | GIVNNTVYDPLQPELDSFKE |
| | | | ELDKYFKNHTSPDVDLGDIS |
| | | | GINASVVNIQKEIDRLNEVA |
| | | | KNLNESLIDLQELGKYEQYI |
| | | | KWP |
| | S1_SARS- | 60 | MFVFLVLLPLVSSQCVNLTT |
| | Cov2 | | RTQLPPAYTNSFTRGVYYPD |
| | | | KVFRSSVLHSTQDLFLPFFS |
| | | | NVTWFHAIHVSGTNGTKRFD |
| | | | NPVLPFNDGVYFASTEKSNI |
| | | | IRGWIFGTTLDSKTQSLLIV |
| | | | NNATNVVIKVCEFQFCNDPF |
| | | | LGVYYHKNNKSWMESEFRVY |
| | | | SSANNCTFEYVSQPFLMDLE |
| | | | GKQGNFKNLREFVFKNIDGY |
| | | | FKIYSKHTPINLVRDLPQGF |
| | | | SALEPLVDLPIGINITRFQT |
| | | | LLALHRSYLTPGDSSSGWTA |
| | | | GAAAYYVGYLQPRTFLLKYN |
| | | | ENGTITDAVDCALDPLSETK |
| | | | CTLKSFTVEKGIYQTSNFRV |
| | | | QPTESIVRFPNITNLCPFGE |
| | | | VFNATRFASVYAWNRKRISN |
| | | | CVADYSVLYNSASFSTFKCY |
| | | | GVSPTKLNDLCFTNVYADSF |
| | | | VIRGDEVRQIAPGQTGKIAD |
| | | | YNYKLPDDFTGCVIAWNSNN |
| | | | LDSKVGGNYNYLYRLFRKSN |
| | | | LKPFERDISTEIYQAGSTPC |
| | | | NGVEGENCYFPLQSYGFQPT |
| | | | NGVGYQPYRVVVLSFELLHA |
| | | | PATVCGPKKSTNLVKNKCVN |
| | | | FNFNGLTGTGVLTESNKKFL |
| | | | PFQQFGRDIADTTDAVRDPQ |
| | | | TLEILDITPCSFGGVSVITP |
| | | | GTNTSNQVAVLYQDVNCTEV |
| | | | PVAIHADQLTPTWRVYSTGS |
| | | | NVFQTRAGCLIGAEHVNNSY |
| | | | ECDIPIGAGICASYQTQTNS |
| | | | P |
| | S1B_SARS- | 61 | RFPNITNLCPFGEVFNATRF |
| | Cov2 | | ASVYAWNRKRISNCVADYSV |
| | | | LYNSASFSTFKCYGVSPTKL |
| | | | NDLCFTNVYADSFVIRGDEV |
| | | | RQIAPGQTGKIADYNYKLPD |
| | | | DFTGCVIAWNSNNLDSKVGG |
| | | | NYNYLYRLFRKSNLKPFERD |
| | | | ISTEIYQAGSTPCNGVEGFN |
| | | | CYFPLQSYGFQPTNGVGYQP |
| | | | YRVVVLSFELLHAPATVCGP |
| | | | KKSTNLVKNKCVNF |
| Aero | Lol p 5 | 62 | ATGCAAGTGGTGCTGGGTTC |
| allergen | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGg |
| | | | gatccCACCACCACCATCAC |
| | | | CACGGTTCCGGACTGAACGA |
| | | | CATCTTCGAGGCTCAAAAGA |
| | | | TCGAGTGGCACGAGggctcg |
| | | | agcGCTGACGCTGGTTACAC |
| | | | TCCAGCTGCTGCAGCTACCC |
| | | | CTGCTACTCCTGCCGCTACA |
| | | | CCAGCTGCCGCTGGCGGAAA |
| | | | AGCTACTACCGACGAGCAAA |
| | | | AGCTGTTGGAGGACGTGAAC |
| | | | GCTGGTTTCAAGGCTGCTGT |
| | | | TGCTGCTGCCGCTAACGCTC |
| | | | CTCCTGCTGACAAGTTCAAG |
| | | | ATCTTCGAAGCTGCTTTCTC |
| | | | CGAGTCCTCCAAGGGCCTGC |
| | | | TGGCTACTTCTGCTGCTAAG |
| | | | GCTCCTGGACTGATCCCCAA |
| | | | GCTGGACACCGCTTACGACG |
| | | | TGGCCTACAAGGCTGCCGAG |
| | | | GCTACTCCAGAGGCTAAGTA |
| | | | CGACGCTTTCGTGACCGCTC |
| | | | TGACCGAGGCTTTGCGTGTG |
| | | | ATCGCTGGTGCTCTGGAAGT |
| | | | GCACGCTGTGAAGCCTGCTA |
| | | | CCGAGGAAGTGCTGGCTGCT |
| | | | AAGATCCCTACTGGCGAGCT |
| | | | GCAGATCGTGGACAAGATCG |
| | | | ACGCTGCCTTCAAGATCGCT |
| | | | GCTACCGCTGCAAACGCTGC |
| | | | TCCCACCAACGATAAGTTCA |
| | | | CCGTGTTCGAGTCCGCTTTC |
| | | | AACAAGGCTCTGAACGAGTG |
| | | | CACCGGTGGTGCTTACGAGA |
| | | | CTTACAAGTTCATCCCATCC |
| | | | TTGGAGGCTGCCGTGAAGCA |
| | | | GGCTTACGCTGCTACTGTAG |
| | | | CTGCTGCTCCCGAAGTGAAG |
| | | | TACGCTGTGTTCGAGGCTGC |
| | | | CCTGACCAAGGCTATCACCG |
| | | | CTATGACTCAGGCTCAGAAG |
| | | | GCTGGCAAGCCTGCTGCGGC |
| | | | TGCTGCTACAGCCGCTGCAA |
| | | | CTGTTGCTACAGCAGCGGCC |
| | | | ACTGCAGCTGCCGTTTTGCC |
| | | | TCCTCCTCTGCTGGTGGTGC |
| | | | AGTCCCTGATCTCCCTGCTG |
| | | | ATCTACTACTAA |
| | Phl p 1 | 63 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGG |
| | | | GATCCCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCATCCCTAAGGTTCCACC |
| | | | TGGTCCTAACATCACCGCTA |
| | | | CCTACGGCGACAAGTGGCTG |
| | | | GACGCTAAGTCCACTTGGTA |
| | | | CGGCAAGCCTACCGGTGCTG |
| | | | GTCCCAAGGATAACGGTGGT |
| | | | GCTTGCGGTTACAAGGACGT |
| | | | GGACAAGCCTCCTTTCTCCG |
| | | | GCATGACCGGTTGCGGCAAC |
| | | | ACCCCTATCTTCAAGTCCGG |
| | | | TCGTGGTTGCGGTTCCTGCT |
| | | | TCGAGATCAAGTGCACCAAG |
| | | | CCTGAGGCCTGCTCTGGCGA |
| | | | ACCTGTGGTGGTGCACATCA |
| | | | CCGACGACAACGAGGAACCT |
| | | | ATCGCTCCCTACGTGTTCGA |
| | | | CCTGTCCGGTCACGCTTTCG |
| | | | GTGCTATGGCTAAGAAGGGC |
| | | | GACGAGCAAAAGCTGCGTTC |
| | | | CGCTGGCGAATTGGAGCTGC |
| | | | AGTTCCGTCGTGTGAAGTGC |
| | | | AAGTACCCCGAGGGCACCAA |
| | | | AGTGACCTTCCACGTCGAGA |
| | | | AGGGTTCTAACCCCAACTAC |
| | | | CTGGCTCTGCTGGTCAAATA |
| | | | CGTGAACGGCGACGGCGACG |
| | | | TGGTGGCTGTGGACATCAAA |
| | | | GAGAAGGGCAAAGACAAGTG |
| | | | GATCGAGCTGAAAGAGTCCT |
| | | | GGGGCGCTATCTGGCGTATC |
| | | | GACACTCCCGACAAGCTGAC |
| | | | CGGTCCTTTCACCGTGCGTT |
| | | | ACACCACCGAAGGTGGCACC |
| | | | AAGACCGAAGCTGAGGACGT |
| | | | GATCCCTGAAGGCTGGAAGG |
| | | | CTGACACCTCCTACGAGTCC |
| | | | AAATAA |
| | Der p 1 | 64 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGG |
| | | | GATCCCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCACTAACGCTTGCTCCAT |
| | | | CAACGGCAACGCTCCCGCTG |
| | | | AAATCGACCTGCGTCAGATG |
| | | | CGTACCGTGACTCCCATCCG |
| | | | TATGCAAGGCGGTTGCGGTT |
| | | | CCTCTTGGGCTTTCTCCGGT |
| | | | GTCGCTGCTACCGAGTCTGC |
| | | | TTACCTGGCTTACCGCAATC |
| | | | AGTCCCTGGACCTGGCTGAG |
| | | | CAAGAACTGGTGGACTGCGC |
| | | | TTCTCAGCACGGTTGTCACG |
| | | | GCGACACTATCCCTCGTGGT |
| | | | ATCGAGTACATCCAGCACAA |
| | | | CGGTGTCGTGCAAGAGTCCT |
| | | | ACTACCGTTACGTGGCCCGC |
| | | | GAGCAGTCTTGCAGACGTCC |
| | | | TAACGCTCAGCGTTTCGGTA |
| | | | TCTCCAACTACTGCCAGATC |
| | | | TACCCTCCTAACGTGAACAA |
| | | | GATCCGCGAGGCTCTGGCTC |
| | | | AGACCCACTCTGCTATCGCT |
| | | | GTGATCATCGGTATCAAGGA |
| | | | CCTGGACGCTTTCCGTCACT |
| | | | ACGACGGTCGTACCATCATC |
| | | | CAGCGTGACAACGGTTACCA |
| | | | GCCTAACTACCACGCTGTCA |
| | | | ACATCGTGGGCTACTCCAAC |
| | | | GCTCAGGGTGTCGACTACTG |
| | | | GATCGTGCGCAACTCCTGGG |
| | | | ACACCAACTGGGGAGACAAC |
| | | | GGCTACGGTTACTTCGCTGC |
| | | | TAACATCGACCTGATGATGA |
| | | | TCGAGGAATACCCCTACGTG |
| | | | GTCATCCTCTAA |
| | Der p 2 | 65 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGG |
| | | | GATCCCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCGACCAAGTGGATGTGAA |
| | | | GGACTGCGCTAACCACGAGA |
| | | | TCAAGAAGGTGCTGGTCCCC |
| | | | GGTTGCCACGGTTCTGAGCC |
| | | | TTGCATCATCCACCGTGGAA |
| | | | AGCCCTTCCAGTTGGAGGCT |
| | | | GTGTTCGAGGCTAACCAGAA |
| | | | CACCAAGACCGCTAAGATCG |
| | | | AAATCAAGGCTTCCATCGAC |
| | | | GGCCTGGAAGTGGACGTGCC |
| | | | CGGTATCGATCCTAACGCTT |
| | | | GCCACTACATGAAGTGCCCT |
| | | | CTGGTCAAGGGCCAGCAGTA |
| | | | CGACATCAAGTACACCTGGA |
| | | | ACGTGCCCAAGATCGCTCCC |
| | | | AAGTCCGAGAACGTGGTGGT |
| | | | CACCGTGAAAGTGATGGGCG |
| | | | ACGACGGTGTCCTGGCTTGC |
| | | | GCTATTGCTACCCACGCCAA |
| | | | GATCCGTGACTAA |
| | Fel d 1 | 66 | ATGAGAGGCGCTCTGCTGGT |
| | | | GCTGGCTCTGCTTGTTACAC |
| | | | AGGCCCTAGGAGTGAAGATG |
| | | | GCCGAGACATGCCCCATCTT |
| | | | CTACGACGTGTTCTTCGCCG |
| | | | TGGCCAACGGCAATGAGCTG |
| | | | CTGCTGGATCTGAGCCTGAC |
| | | | CAAAGTGAACGCCACCGAGC |
| | | | CTGAGAGAACCGCCATGAAG |
| | | | AAGATCCAGGACTGCTACGT |
| | | | GGAAAACGGCCTGATCAGCA |
| | | | GAGTGCTGGACGGCCTGGTC |
| | | | ATGACAACCATCAGCAGCTC |
| | | | CAAGGACTGCATGGGCGAAG |
| | | | CCGTGCAGAACACCGTGGAA |
| | | | GATCTGAAGCTGAACACCCT |
| | | | GGGCCGCGAGATTTGTCCCG |
| | | | CCGTGAAGAGAGATGTGGAC |
| | | | CTGTTCCTGACCGGCACACC |
| | | | CGACGAGTATGTGGAACAGG |
| | | | TGGCCCAGTATAAGGCCCTG |
| | | | CCTGTGGTGCTGGAAAACGC |
| | | | CAGAATCCTGAAGAACTGCG |
| | | | TGGACGCCAAGATGACCGAA |
| | | | GAGGACAAAGAGAATGCCCT |
| | | | GAGCCTGCTGGACAAGATCT |
| | | | ACACAAGCCCTCTGTGCGGA |
| | | | TCCGGCCTGAACGACATCTT |
| | | | CGAGGCCCAGAAAATCGAGT |
| | | | GGCACGAGGGCCACCACCAT |
| | | | CATCACCATTGA |
| Food allergen | Ara h 2 | 67 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGG |
| | | | GATCCCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCAGACAGCAATGGGAATT |
| | | | GCAAGGCGACCGTCGTTGCC |
| | | | AGTCTCAGCTCGAACGTGCT |
| | | | AACCTGAGGCCTTGCGAGCA |
| | | | GCACCTGATGCAGAAGATCC |
| | | | AACGCGACGAGGACTCCTAC |
| | | | GGTCGTGACCCTTACAGCCC |
| | | | TTCTCAGGATCCATACTCAC |
| | | | CCTCTCAAGACCCTGACCGT |
| | | | CGCGACCCCTATTCTCCCTC |
| | | | TCCTTACGATCGTCGCGGTG |
| | | | CTGGTTCCTCTCAGCACCAA |
| | | | GAAAGGTGCTGCAACGAACT |
| | | | GAACGAGTTCGAGAACAACC |
| | | | AGCGTTGCATGTGCGAGGCT |
| | | | CTGCAGCAGATCATGGAAAA |
| | | | CCAGTCCGACCGTCTGCAGG |
| | | | GTCGTCAGCAAGAGCAGCAG |
| | | | TTCAAGCGCGAGCTGCGTAA |
| | | | CCTGCCTCAGCAATGCGGAC |
| | | | TGCGTGCTCCTCAACGTTGC |
| | | | GACCTGGAAGTGGAATCCGG |
| | | | TGGACGTGACCGTTACTAA |
| | Pen m 1 | 68 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGG |
| | | | GATCCCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCGACGCTATCAAGAAAAA |
| | | | GATGCAGGCCATGAAGCTGG |
| | | | AAAAGGACAACGCTATGGAC |
| | | | CGTGCTGACACCCTGGAACA |
| | | | GCAGAACAAAGAGGCTAACA |
| | | | ACCGCGCTGAGAAGTCCGAG |
| | | | GAAGAGGTGCACAACCTGCA |
| | | | AAAGCGTATGCAGCAGTTGG |
| | | | AGAACGACCTGGACCAGGTG |
| | | | CAAGAGTCCCTGCTGAAGGC |
| | | | TAACATCCAGCTGGTCGAGA |
| | | | AGGACAAGGCTCTGTCCAAC |
| | | | GCTGAGGGCGAAGTGGCTGC |
| | | | TCTGAACCGTCGTATCCAGC |
| | | | TGCTGGAAGAGGACTTGGAG |
| | | | CGTTCCGAGGAACGTCTGAA |
| | | | CACCGCTACCACCAAGCTGG |
| | | | CTGAGGCTTCTCAGGCTGCT |
| | | | GACGAGTCTGAGCGTATGCG |
| | | | CAAGGTGCTGGAAAACCGTT |
| | | | CTCTGTCCGACGAGGAACGC |
| | | | ATGGACGCTTTGGAGAACCA |
| | | | GCTGAAAGAGGCTCGTTTCC |
| | | | TGGCTGAAGAGGCTGACCGT |
| | | | AAATACGACGAGGTGGCCCG |
| | | | CAAGCTCGCTATGGTGGAAG |
| | | | CTGACTTGGAACGCGCTGAG |
| | | | GAAAGGGCTGAGACTGGCGA |
| | | | GTCCAAGATCGTGGAACTCG |
| | | | AAGAGGAACTGCGTGTCGTG |
| | | | GGCAACAACCTGAAGTCCCT |
| | | | GGAAGTGTCCGAAGAGAAGG |
| | | | CCAACCAGCGCGAAGAGGCT |
| | | | TACAAAGAGCAGATCAAGAC |
| | | | CCTGACCAACAAGCTGAAGG |
| | | | CCGCTGAGGCTCGTGCTGAG |
| | | | TTCGCTGAACGTTCCGTGCA |
| | | | AAAGCTGCAGAAAGAGGTGG |
| | | | ACCGTTTGGAGGACGAGCTG |
| | | | GTCAACGAGAAAGAGAAGTA |
| | | | CAAGTCCATCACCGACGAGT |
| | | | TGGACCAGACCTTCTCCGAG |
| | | | CTGTCCGGTTACTAA |
| SARS-CoV2 | Nucleo- | 69 | ATGGTCCTGAGCCTGCTGTA |
| | capsid | | CCTGQTTACAGCCCTGCCTG |
| | | | GCATCTTAAGCAGCGACAAC |
| | | | GGCCCTCAGAACCAGCGGAA |
| | | | TGCCCCTAGAATCACCTTTG |
| | | | GCGGCCCTAGCGATAGCACC |
| | | | GGCAGCAATCAGAATGGCGA |
| | | | GAGAAGCGGCGCCAGAAGCA |
| | | | AGCAGAGAAGGCCTCAAGGC |
| | | | CTGCCTAACAACACCGCCAG |
| | | | CTGGTTCACAGCCCTGACAC |
| | | | AGCACGGCAAAGAGGACCTG |
| | | | AAGTTCCCTAGAGGACAGGG |
| | | | CGTGCCCATCAACACCAACA |
| | | | GCAGCCCCGATGACCAGATC |
| | | | GGCTACTACAGACGGGCCAC |
| | | | CAGAAGAATCAGAGGCGGCG |
| | | | ACGGCAAGATGAAGGATCTG |
| | | | AGCCCCAGATGGTACTTCTA |
| | | | CTACCTCGGCACAGGACCCG |
| | | | AAGCCGGACTTCCTTATGGC |
| | | | GCCAACAAGGACGGCATCAT |
| | | | CTGGGTTGCAACAGAAGGCG |
| | | | CCCTGAACACCCCTAAGGAC |
| | | | CACATCGGCACCAGAAATCC |
| | | | CGCCAACAATGCCGCCATTG |
| | | | TGCTGCAGTTGCCTCAGGGC |
| | | | ACAACACTGCCCAAGGGCTT |
| | | | TTACGCCGAGGGAAGCAGAG |
| | | | GCGGATCTCAGGCCTCTAGC |
| | | | AGAAGCAGCAGCCGGTCCAG |
| | | | AAACAGCTCCCGGAATAGCA |
| | | | CACCTGGCAGCAGCAGAGGC |
| | | | ACAAGCCCTGCTAGAATGGC |
| | | | TGGCAACGGCGGAGATGCTG |
| | | | CTCTGGCACTTCTGCTGCTC |
| | | | GACCGGCTGAATCAGCTGGA |
| | | | AAGCAAGATGAGCGGCAAGG |
| | | | GCCAGCAGCAACAGGGCCAG |
| | | | ACCGTGACAAAAAAGTCTGC |
| | | | CGCCGAGGCCAGCAAGAAGC |
| | | | CCAGACAGAAAAGAACCGCC |
| | | | ACCAAGGCCTACAACGTGAC |
| | | | CCAGGCCTTTGGCAGAAGAG |
| | | | GCCCTGAGCAGACCCAGGGC |
| | | | AATTTCGGCGATCAAGAGCT |
| | | | GATCAGACAGGGCACCGACT |
| | | | ACAAGCACTGGCCTCAGATC |
| | | | GCCCAGTTTGCCCCTTCTGC |
| | | | CAGCGCCTTCTTTGGCATGA |
| | | | GCCGGATCGGCATGGAAGTG |
| | | | ACCCCATCTGGCACCTGGCT |
| | | | GACATACACAGGCGCCATCA |
| | | | AGCTGGACGACAAGGACCCC |
| | | | AACTTCAAGGACCAAGTGAT |
| | | | CCTGCTGAACAAGCACATCG |
| | | | ACGCCTACAAGACATTCCCT |
| | | | CCAACCGAGCCTAAGAAGGA |
| | | | CAAGAAGAAGAAGGCCGACG |
| | | | AGACACAGGCCCTGCCTCAG |
| | | | CGCCAGAAAAAGCAGCAGAC |
| | | | TGTGACACTGCTGCCCGCTG |
| | | | CCGACCTGGACGATTTTTCT |
| | | | AAGCAGCTGCAGCAGAGCAT |
| | | | GAGCAGCGCCGATTCTACAC |
| | | | AGGCTGGATCCGGACTGAAC |
| | | | GACATCTTCGAAGCCCAGAA |
| | | | AATCGAGTGGCACGAGGGCC |
| | | | ACCACCATCATCACCATTGA |
| SARS-CoV2 | S1 | 70 | ATGGTCCTGAGCCTGCTGTA |
| | | | CCTGCTTACAGCCCTGCCTG |
| | | | GCATCTTAAGCGTGAACCTG |
| | | | ACCACCAGAACACAGCTGCC |
| | | | TCCAGCCTACACCAACAGCT |
| | | | TCACCAGAGGCGTGTACTAC |
| | | | CCCGACAAGGTGTTCAGATC |
| | | | CAGCGTGCTGCACTCTACCC |
| | | | AGGACCTGTTCCTGCCTTTC |
| | | | TTCAGCAACGTGACCTGGTT |
| | | | CCACGCCATCCACGTGTCCG |
| | | | GCACCAATGGCACCAAGAGA |
| | | | TTCGACAACCCCGTGCTGCC |
| | | | CTTCAACGACGGGGTGTACT |
| | | | TTGCCAGCACCGAGAAGTCC |
| | | | AACATCATCAGAGGCTGGAT |
| | | | CTTCGGCACCACACTGGACA |
| | | | GCAAGACCCAGAGCCTGCTG |
| | | | ATCGTGAACAACGCCACCAA |
| | | | CGTGGTCATCAAAGTGTGCG |
| | | | AGTTCCAGTTCTGCAACGAC |
| | | | CCCTTCCTGGGCGTCTACTA |
| | | | CCACAAGAACAACAAGAGCT |
| | | | GGATGGAAAGCGAGTTCCGG |
| | | | GTGTACAGCAGCGCCAACAA |
| | | | CTGCACCTTCGAGTACGTGT |
| | | | CCCAGCCTTTCCTGATGGAC |
| | | | CTGGAAGGCAAGCAGGGCAA |
| | | | CTTCAAGAACCTGCGCGAGT |
| | | | TCGTGTTCAAGAACATCGAC |
| | | | GGCTACTTCAAGATCTACAG |
| | | | CAAGCACACCCCTATCAACC |
| | | | TCGTGCGGGATCTGCCTCAG |
| | | | GGCTTCTCTGCTCTGGAACC |
| | | | CCTGGTGGATCTGCCCATCG |
| | | | GCATCAACATCACCCGGTTT |
| | | | CAGACACTGCTGGCCCTGCA |
| | | | CAGAAGCTACCTGACACCTG |
| | | | GCGATAGCAGCTCTGGATGG |
| | | | ACAGCTGGCGCCGCTGCCTA |
| | | | CTATGTGGGATACCTGCAGC |
| | | | CTCGGACCTTCCTGCTGAAG |
| | | | TACAACGAGAACGGCACCAT |
| | | | CACCGACGCCGTGGATTGTG |
| | | | CCCTTGATCCTCTGAGCGAG |
| | | | ACAAAGTGCACCCTGAAGTC |
| | | | CTTCACCGTGGAAAAGGGCA |
| | | | TCTACCAGACCAGCAACTTC |
| | | | CGGGTGCAGCCCACCGAATC |
| | | | CATCGTGCGGTTCCCCAATA |
| | | | TCACCAATCTGTGCCCCTTC |
| | | | GGCGAGGTGTTCAATGCCAC |
| | | | CAGATTCGCCTCTGTGTACG |
| | | | CCTGGAACCGGAAGCGGATC |
| | | | AGCAATTGCGTGGCCGACTA |
| | | | CTCCGTGCTGTACAACTCCG |
| | | | CCAGCTTCAGCACCTTCAAG |
| | | | TGCTACGGCGTGTCCCCTAC |
| | | | CAAGCTGAACGACCTGTGCT |
| | | | TCACAAACGTGTACGCCGAC |
| | | | AGCTTCGTGATCCGGGGAGA |
| | | | TGAAGTGCGGCAGATTGCCC |
| | | | CTGGACAGACAGGCAAGATC |
| | | | GCCGACTACAACTACAAGCT |
| | | | GCCCGACGACTTCACCGGCT |
| | | | GTGTGATTGCCTGGAACAGC |
| | | | AACAACCTGGACTCCAAAGT |
| | | | CGGCGGCAACTACAATTACC |
| | | | TGTACCGGCTGTTCCGGAAG |
| | | | TCCAATCTGAAGCCCTTCGA |
| | | | GCGGGACATCTCCACCGAGA |
| | | | TCTATCAGGCCGGCAGCACC |
| | | | CCTTGTAACGGCGTGGAAGG |
| | | | CTTCAACTGCTACTTCCCAC |
| | | | TGCAGTCCTACGGCTTTCAG |
| | | | CCCACAAATGGCGTGGGCTA |
| | | | CCAGCCTTACAGAGTGGTGG |
| | | | TGCTGAGCTTCGAGCTGCTG |
| | | | CATGCTCCTGCCACAGTGTG |
| | | | CGGCCCTAAGAAAAGCACCA |
| | | | ATCTCGTGAAGAACAAATGC |
| | | | GTGAACTTCAACTTCAACGG |
| | | | CCTGACCGGCACCGGCGTGC |
| | | | TGACAGAGAGCAACAAGAAG |
| | | | TTCCTGCCATTCCAGCAGTT |
| | | | CGGCCGGGATATCGCCGATA |
| | | | CCACAGACGCCGTTAGAGAT |
| | | | CCCCAGACACTGGAAATCCT |
| | | | GGACATCACCCCTTGCAGCT |
| | | | TCGGCGGAGTGTCTGTGATC |
| | | | ACCCCTGGCACCAACACCAG |
| | | | CAATCAGGTGGCAGTGCTGT |
| | | | ACCAGGACGTGAACTGTACC |
| | | | GAAGTGCCCGTGGCCATTCA |
| | | | CGCCGATCAGCTGACACCTA |
| | | | CATGGCGGGTGTACTCCACC |
| | | | GGCAGCAATGTGTTTCAGAC |
| | | | CAGAGCCGGCTGTCTGATCG |
| | | | GAGCCGAGCACGTGAACAAT |
| | | | AGCTACGAGTGCGACATCCC |
| | | | CATCGGCGCTGGCATCTGTG |
| | | | CCAGCTATCAGACCCAGACG |
| | | | AATTCTCCCGGATCCGGCCT |
| | | | GAACGACATCTTCGAGGCCC |
| | | | AGAAAATCGAGTGGCACGAG |
| | | | GGCCACCACCATCATCACCA |
| | | | TTGA |
| SARS-CoV2 | S1B | 71 | ATGAGAGGCGCTCTGCTGGT |
| | | | GCTGGCTCTGCTTGTTACAC |
| | | | AAGCTTTAGGACGGTTCCCC |
| | | | AATATCACCAATCTGTGCCC |
| | | | CTTCGGCGAGGTGTTCAATG |
| | | | CCACCAGATTCGCCTCTGTG |
| | | | TACGCCTGGAACCGGAAGCG |
| | | | GATCAGCAATTGCGTGGCCG |
| | | | ACTACTCCGTGCTGTACAAC |
| | | | TCCGCCAGCTTCAGCACCTT |
| | | | CAAGTGCTACGGCGTGTCCC |
| | | | CTACCAAGCTGAACGACCTG |
| | | | TGCTTCACAAACGTGTACGC |
| | | | CGACAGCTTCGTGATCCGGG |
| | | | GAGATGAAGTGCGGCAGATT |
| | | | GCCCCTGGACAGACAGGCAA |
| | | | GATCGCCGACTACAACTACA |
| | | | AGCTGCCCGACGACTTCACC |
| | | | GGCTGTGTGATTGCCTGGAA |
| | | | CAGCAACAACCTGGACTCCA |
| | | | AAGTCGGCGGCAACTACAAT |
| | | | TACCTGTACCGGCTGTTCCG |
| | | | GAAGTCCAATCTGAAGCCCT |
| | | | TCGAGCGGGACATCTCCACC |
| | | | GAGATCTATCAGGCCGGCAG |
| | | | CACCCCTTGTAACGGCGTGG |
| | | | AAGGCTTCAACTGCTACTTC |
| | | | CCACTGCAGTCCTACGGCTT |
| | | | TCAGCCCACAAATGGCGTGG |
| | | | GCTACCAGCCTTACAGAGTG |
| | | | GTGGTGCTGAGCTTCGAGCT |
| | | | GCTGCATGCTCCTGCCACAG |
| | | | TGTGCGGCCCTAAGAAAAGC |
| | | | ACCAATCTCGTGAAGAACAA |
| | | | ATGCGTGAACTTCGGATCCG |
| | | | GCCTGAACGACATCTTCGAA |
| | | | GCTCAGAAAATCGAGTGGCA |
| | | | CGAGGGCCACCACCATCATC |
| | | | ACCATTGA |
| Avi-tag | BirA Tag | 72 | GLNDIFEAQKIEWHE |
| | 2/AviTag | | |
| Fel d 1 | mammal Fel | 73 | MRGALLVLALLVTQALG |
| leader | d 1 leader | | |
| Leader | Insect Api | 74 | MQVVLGSLFLLLLSTSHG |
| sequence | m 1 short | | |
| | leader | | |
| Leader | Mammal Fel | 75 | MRGALLVLALLVTQALG |
| sequence | d 1 leader | | |
| Vaccine | HA_A- | 76 | MKAILVVLLYTFTTANADTL |
| antigen | Brisbane- | | CIGYHANNSTDTVDTVLEKN |
| | 2018 | | VTVTHSVNLLEDKHNGKLCK |
| | | | LGGVAPLHLGKCNIAGWILG |
| | | | NPECESLSTARSWSYIVETS |
| | | | NSDNGTCY(F)FPGDFINYE |
| | | | ELREQLSSVSSFERFEIFPK |
| | | | TSSWPNHDSNKGVTAACPHA |
| | | | GAKSFYKNLIWLVKKGNSYP |
| | | | KLNQTYINDKGKEVLVLWGI |
| | | | HHPPTTADQQSLYQNADAYV |
| | | | FVGTSRYSKKFKPEIATRPK |
| | | | VRDREGRMNYYWTLVEPGDK |
| | | | ITFEATGNLVVPRYAFTMER |
| | | | NAGSGIIISDTPVHDCNTTC |
| | | | QTAEGAINTSLPFQNVHPVT |
| | | | IGKCPKYVKSTKLRLATGLR |
| | | | NVPSIQSRGLFGAIAGFIEG |
| | | | GWTGMVDGWYGYHHQNEQGS |
| | | | GYAADLKSTQNAIDKITNKV |
| | | | NSVIEKMNTQFTAVGKEFNH |
| | | | LEKRIENLNKKVDDGFLDIW |
| | | | TYNAELLVLLENERTLDYHD |
| | | | SNVKNLYEKVRNQLKNNAKE |
| | | | IGNGCFEFYHKCDNTCMESV |
| | | | KNGTYDYPKYSEEAKLNREK |
| | | | IDGSGYIPEAPRDGQAYVRK |
| | | | DGEWVLLSTFLGS |
| Insect venom | Api m 3 | 77 | ELKQINVIFRHGDRIPDEKN |
| allergen | | | EMYPKDPYLYYDFYPLERGE |
| | | | LTNSGKMREYQLGQFLRERY |
| | | | GDFLGDIYTEESVSALSSFY |
| | | | DRTKMSLQLVLAALYPPNKL |
| | | | QQWNEDLNWQPIATKYLRRY |
| | | | EDNIFLPEDCLLFTIELDRV |
| | | | LESPRGK |
| | | | YEFSKYDKLKKKLEEWTGKN |
| | | | ITTPWDYYYIYHTLVAEQSY |
| | | | GLTLPSWTNNIFPRGELFDA |
| | | | TVFTYNITNSTPLLKKLYGG |
| | | | PLLRIFTKHMLDVVSGTQKK |
| | | | KRKIYLFSGHESNIASVLHA |
| | | | LQLYYPHVPEYSSSIIMELH |
| | | | NIEGTHYVKIVYYLGIPSEA |
| | | | RELQLPGCEVLCPLYKYLQL |
| | | | IENVIPSNEELICDKRFVDE |
| | | | SANNLSIEELDFVKLNLIRI |
| | | | AGTENK |
| Insect venom | Pol a 1 | 78 | MSPDCTFNEKDIVFYVYSRD |
| allergen | | | KRDGIILKKETLTNYDLFTK |
| | | | STISKQVVFLIHGFLSTGNN |
| | | | ENFVAMSKALIEKDDFLVIS |
| | | | VDWKKGACNAFASTKDALGY |
| | | | SKAVGNTRHVGKFVADFTKL |
| | | | LVEKYKVLISNIRLIGHSLG |
| | | | AHTSGFAGKEVQKLKLGKYK |
| | | | EIIGLDPAGPYFHRSDCPDR |
| | | | LCVTDAEYVQVIHTSIILGV |
| | | | YYNVGSVDFYVNYGKNQPGC |
| | | | NEPSCSHTKAVKYLTECIKH |
| | | | ECCLIGTPWKKYFSTPKPIS |
| | | | QCRGDTCVCVGLNAKSYPAR |
| | | | GAFYAPVEANAPYCHNEGIK |
| | | | L |
| | Pol d 1 | 79 | ADDLTTLRNGTLDRGITPDC |
| | | | TFNEKDIELHVYSRDKRNGI |
| | | | ILKKEILKNYDLFQKSQISH |
| | | | QIAILIHGFLSTGNNENFDA |
| | | | MAKALIEIDNFLVISVDWKK |
| | | | GACNAFASTNDVLGYSQAVG |
| | | | NTRHVGKYVADFTKLLVEQY |
| | | | KVPMSNIRLIGHSLGAHTSG |
| | | | FAGKEVQRLKLGKYKEIIGL |
| | | | DPAGPSFLTNKCPNRLCETD |
| | | | AEYVQAIHTSAILGVYYNVG |
| | | | SVDFYVNYGKSQPGCSEPSC |
| | | | SHTKAVKYLTECIKRECCLI |
| | | | GTPWKSYFSTPKPISQCKRD |
| | | | TCVCVGLNAQSYPAKGSFYV |
| | | | PVDKDAPYCHNEGIKL |
| | Pol d 5 | 80 | NDYCKIKCSSGVHTVCQYGE |
| | | | STKPSKNCAGKLIKSVGPTE |
| | | | EEKKLIVEEHNRFRQKVAKG |
| | | | LETRGNPGPQPAASNMNNLV |
| | | | WNDELAKIAQVWASQCQILV |
| | | | HDKCRNTEKYQVGQNIAYAG |
| | | | SSNHFPSVTKLIQLWENEVK |
| | | | DFNYNTGITNKNFGKVGHYT |
| | | | QMVWGNTKEVGCGSLKYVEK |
| | | | NMQIHYLICNYGPAGNYLGQ |
| | | | PIYTKK |
| | Soll 1 | 81 | EPDPGVVEYLKQSCVYGNSS |
| | | | YINVYLYNSRFQGKNLGNQQ |
| | | | SCQDINASLPVVFITHGFTS |
| | | | SAQVSTFKDLANAFVQKGHT |
| | | | AFIVDWSEAACTDGLPGVQF |
| | | | AEYNAAASNTYDIGQLMAKY |
| | | | TVDLMNKCKIPLNNIQYVGH |
| | | | SLGSHVCGFAAKHVKKLINK |
| | | | TMPYILALDPADPSFGSNKC |
| | | | GERICKSDAKRIVVFKTSIL |
| | | | GIGENIIGHLLIVFDGGKSQ |
| | | | PACSWYDVPCSHSESIVYAT |
| | | | GMVSGRCQHLAVPWTAQQRI |
| | | | NPIQWKFWRVFTSNIPAYPT |
| | | | SDTTNCVVLNTNVFKNDNTF |
| | | | EGEYHAFPDCARNLFKCRQQ |
| | Myr p 1 | 82 | KDLADPESEAVGFADAFGEA |
| | | | DAVGEADPNAGLGSVFGRLA |
| | | | RILGRVIPKVAKKLGPKVAK |
| | | | VLPKVMKEAIPMAVEMAKSQ |
| | | | EEQQPQ |
| | Myr p 2 | 83 | KALADPESDAVGFADAVGEA |
| | | | DPIDWKKVDWKKVSKKTCKV |
| | | | MLKACKFL |
| | Myr p 3 | 84 | PNVKAKALADPESDAVGFAD |
| | | | AVGEADPFDITKLNIKKLTK |
| | | | ATCKVISKGASMCKVLFDKK |
| | | | KQE |
| | Ara h 9 | 85 | ISCGQVNSALAPCIPFLTKG |
| | | | GAPPPACCSGVRGLLGALRT |
| | | | TADRQAACNCLKAAAGSLRG |
| | | | LNQGNAAALPGRCGVSIPYK |
| | | | ISTSTNCATIKF |
| | Tri a 19 | 86 | SRLLSPRGKELHTPQEQFPQ |
| | | | QQQFPQPQQFPQQQIPQQHQ |
| | | | IPQQPQQFPQQQQFLQQQQI |
| | | | PQQQIPQQHQIPQQPQQFPQ |
| | | | QQQFPQQHQSPQQQFPQQQF |
| | | | PQQKLPQQEFPQQQISQQPQ |
| | | | QLPQQQQIPQQPQQFLQQQQ |
| | | | FPQQQPPQQHQFPQQQLPQQ |
| | | | QQIPQQQQIPQQPQQIPQQQ |
| | | | QIPQQPQQFPQQQFPQQQFP |
| | | | QQQFPQQEFPQQQQFPQQQI |
| | | | ARQPQQLPQQQQIPQQPQQF |
| | | | PQQQQFPQQQSPQQQQFPQQ |
| | | | QFPQQQQLPQKQFPQPQQIP |
| | | | QQQQIPQQPQQFPQQQFPQQ |
| | | | QQFPQQQEFPQQQFPQQQFH |
| | | | QQQLPQQQFPQQQFPQQQFP |
| | | | QQQQFPQQQQLTQQQFPRPQ |
| | | | QSPEQQQFPQQQFPQQPPQQ |
| | | | FPQQQFPIPYPPQQSEEPSP |
| | | | YQQYPQQQPSGSDVISISGL |
| | Der p 1 | 87 | ATGCAAGTGGTGCTGGGTTC |
| | | | CCTGTTCCTGCTGCTGCTGT |
| | | | CTACCTCTCACggatccCAC |
| | | | CACCACCATCACCACGGTTC |
| | | | CGGACTGAACGACATCTTCG |
| | | | AGGCTCAAAAGATCGAGTGG |
| | | | CACGAGGGCTCGAGCACTAA |
| | | | CGCTTGCTCCATCAACGGCA |
| | | | ACGCTCCCGCTGAAATCGAC |
| | | | CTGCGTCAGATGCGTACCGT |
| | | | GACTCCCATCCGTATGCAAG |
| | | | GCGGTTGCGGTTCCTCTTGG |
| | | | GCTTTCTCCGGTGTCGCTGC |
| | | | TACCGAGTCTGCTTACCTGG |
| | | | CTTACCGCAATCAGTCCCTG |
| | | | GACCTGGCTGAGCAAGAACT |
| | | | GGTGGACTGCGCTTCTCAGC |
| | | | ACGGTTGTCACGGCGACACT |
| | | | ATCCCTCGTGGTATCGAGTA |
| | | | CATCCAGCACAACGGTGTCG |
| | | | TGCAAGAGTCCTACTACCGT |
| | | | TACGTGGCCCGCGAGCAGTC |
| | | | TTGCAGACGTCCTAACGCTC |
| | | | AGCGTTTCGGTATCTCCAAC |
| | | | TACTGCCAGATCTACCCTCC |
| | | | TAAC |
| | | | GTGAACAAGATCCGCGAGGC |
| | | | TCTGGCTCAGACCCACTCTG |
| | | | CTATCGCTGTGATCATCGGT |
| | | | ATCAAGGACCTGGACGCTTT |
| | | | CCGTCACTACGACGGTCGTA |
| | | | CCATCATCCAGCGTGACAAC |
| | | | GGTTACCAGCCTAACTACCA |
| | | | CGCTGTCAACATCGTGGGCT |
| | | | ACTCCAACGCTCAGGGTGTC |
| | | | GACTACTGGATCGTGCGCAA |
| | | | CTCCTGGGACACCAACTGGG |
| | | | GAGACAACGGCTACGGTTAC |
| | | | TTCGCTGCTAACATCGACCT |
| | | | GATGATGATCGAGGAATACC |
| | | | CCTACGTGGTCATCCTCTAA |
| | Tri a 19 | 88 | ATGCAAGTGGTGCTGGGTTC |
| | | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGg |
| | | | gatccCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCTCCCGTTTGCTGTCCCC |
| | | | TCGTGGAAAAGAGCTGCACA |
| | | | CCCCTCAAGAGCAGTTCCCT |
| | | | CAGCAGCAGCAATTCCCACA |
| | | | GCCTCAACAGTTCCCACAAC |
| | | | AGCAAATCCCTCAACAGCAT |
| | | | CAGATCCCGCAGCAGCCCCA |
| | | | ACAATTTCCCCAGCAACAGC |
| | | | AGTTCTTGCAGCAACAACAG |
| | | | ATTCCACAGCAGCAGATTCC |
| | | | TCAGCAACACCAAATTCCGC |
| | | | AACAACCCCAGCAGTTTCCG |
| | | | CAACAGCAACAGTTTCCCCA |
| | | | ACAACATCAATCTCCCCAGC |
| | | | AGCAATTTCCGCAGCAGCAG |
| | | | TTCCCTCAACAGAAGCTGCC |
| | | | CCAGCAAGAGTTCCCGCAGC |
| | | | AACAAATCAGCCAGCAGCCT |
| | | | CAGCAATTGCCTCAGCAGCA |
| | | | ACAAATCCCTCAGCAACCAC |
| | | | AGCAGTTCCTCCAACAACAG |
| | | | CAATTTCCACAACAACAGCC |
| | | | ACCTCAGCAGCACCAGTTTC |
| | | | CGCAGCAACAGCTGCCTCAG |
| | | | CAACAGCAGATCCCACAACA |
| | | | ACAACAAATTCCTCAGCAGC |
| | | | CCCAGCAAATCCCACAGCAG |
| | | | CAGCAAATTCCCCAACAGCC |
| | | | ACAGCAATTCCCTCAACAAC |
| | | | AGTTCCCACAGCAACAATTT |
| | | | CCACAGCAACAGTTCCCGCA |
| | | | GCAAGAGTTCCCACAGCAGC |
| | | | AACAGTTCCCTCAGCAACAG |
| | | | ATCGCTCGTCAGCCACAGCA |
| | | | GCTGCCACAACAGCAGCAAA |
| | | | TTCCACAGCAACCCCAACAA |
| | | | TTCCCACAACAACAACAGTT |
| | | | TCCGCAACAGCAGTCTCCCC |
| | | | AGCAGCAGCAGTTTCCCCAG |
| | | | CAGCAGTTTCCACAACAGCA |
| | | | GCAGCTGCCTCAGAAGCAAT |
| | | | TCCCTCAGCCACAGCAGATC |
| | | | CCTCAGCAGCAGCAGATCCC |
| | | | TCAACAGCCCCAACAGTTCC |
| | | | CGCAGCAGCAGTTTCCCCAA |
| | | | CAACAACAGTTCCCTCAGCA |
| | | | GCAAGAGTTTCCTCAACAGC |
| | | | AATTTCCTCAGCAACAATTC |
| | | | CACCAGCAGCAACTGCCCCA |
| | | | ACAACAATTCCCGCAACAGC |
| | | | AATTCCCGCAGCAGCAATTT |
| | | | CCTCAGCAACAGCAATTCCC |
| | | | ACAACAGCAGCAACTGACCC |
| | | | AGCAACAGTTTCCTCGTCCT |
| | | | CAGCAGTCCCCTGAACAACA |
| | | | GCAGTTTCCACAGCAGCAAT |
| | | | TCCCGCAACAGCCTCCTCAG |
| | | | CAGTTCCCGCAACAACAGTT |
| | | | TCCTATTCCTTATCCTCCTC |
| | | | AGCAATCCGAGGAACCCTCA |
| | | | CCTTACCAGCAGTACCCACA |
| | | | GCAACAGCCCTCCGGTTCCG |
| | | | ACGTGATCTCCATCTCTGGT |
| | | | CTGTAA |
| | Tri a 19 | 89 | ATGCAAGTGGTGCTGGGTTC |
| | short | | CCTGTTCCTGCTGCTGCTGT |
| | | | CTACCTCTCACGGCTGGCAA |
| | | | ATCCGTGACCGTATCGGCGA |
| | | | CAACGAGCTGGAAGAGAGGg |
| | | | gatccCACCACCACCATCAC |
| | | | CACGGTTCCGGACTGAACGA |
| | | | CATCTTCGAGGCTCAAAAGA |
| | | | TCGAGTGGCACGAGGGCTCG |
| | | | AGCCAACAACAGTTTCCGCA |
| | | | ACAGCAGTCTCCCCAGCAGC |
| | | | AGCAGTTTCCCCAGCAGCAG |
| | | | TTTCCACAACAGCAGCAGCT |
| | | | GCCTCAGAAGCAATTCCCTC |
| | | | AGCCACAGCAGATCCCTCAG |
| | | | CAGCAGCAGATCCCTCAACA |
| | | | GCCCCAACAGTTCCCGCAGC |
| | | | AGCAGTTTCCCCAACAACAA |
| | | | CAGTTCCCTCAGCAGCAAGA |
| | | | GTTTCCTCAACAGCAATTTC |
| | | | CTCAGCAACAATTCCACCAG |
| | | | CAGCAACTGCCCCAACAACA |
| | | | ATTCCCGCAACAGCAATTCC |
| | | | CGCAGCAGCAATTTCCTCAG |
| | | | CAACAGCAATTCCCACAACA |
| | | | GCAGCAACTGACCCAGCAAC |
| | | | AGTTTCCTCGTCCTCAGCAG |
| | | | TCCCCTGAACAACAGCAGTT |
| | | | TCCACAGCAGCAATTCCCGC |
| | | | AACAGCCTCCTCAGCAGTTC |
| | | | CCGCAACAACAGTTTCCTAT |
| | | | TCCTTATCCTCCTCAGCAAT |
| | | | CCGAGGAACCCTCACCTTAC |
| | | | CAGCAGTACCCACAGCAACA |
| | | | GCCCTCCGGTTCCGACGTGA |
| | | | TCTCCATCTCTGGTCTGTAA |
| | HA_A_Brisb | 90 | ATGAAGGCCATCCTGGTGGT |
| | ane-2018 | | GCTGCTGTACACCTTCACCA |
| | | | CCGCCAACGCCGACACACTG |
| | | | TGTATCGGCTACCACGCCAA |
| | | | CAACAGCACCGACACCGTGG |
| | | | ATACCGTGCTGGAAAAGAAC |
| | | | GTGACCGTGACACACAGCGT |
| | | | GAACCTGCTGGAAGATAAGC |
| | | | ACAACGGCAAGCTGTGCAAG |
| | | | CTCGGCGGAGTTGCTCCTCT |
| | | | GCACCTGGGCAAGTGTAATA |
| | | | TCGCCGGCTGGATTCTGGGC |
| | | | AACCCCGAGTGTGAAAGCCT |
| | | | GAGCACAGCCAGATCCTGGT |
| | | | CCTACATCGTGGAAACCAGC |
| | | | AACAGCGACAACGGCACATG |
| | | | CTTCCCCGGCGACTTCATCA |
| | | | ACTACGAGGAACTGCGGGAA |
| | | | CAGCTGAGCAGCGTGTCCAG |
| | | | CTTCGAGAGATTCGAGATCT |
| | | | TCCCCAAGACCAGCAGCTGG |
| | | | CCCAACCACGATTCTAACAA |
| | | | GGGCGTGACAGCCGCCTGTC |
| | | | CTCATGCCGGCGCTAAGAGC |
| | | | TTCTACAAGAACCTGATCTG |
| | | | GCTCGTGAAGAAGGGCAACA |
| | | | GCTACCCCAAGCTGAACCAG |
| | | | ACCTACATCAACGACAAGGG |
| | | | CAAAGAGGTGCTGGTCCTCT |
| | | | GGGGCATCCACCATCCACCT |
| | | | ACAACAGCCGATCAGCAGAG |
| | | | CCTGTACCAGAACGCCGATG |
| | | | CCTATGTGTTCGTGGGCACC |
| | | | AGCCGGTACAGCAAGAAGTT |
| | | | CAAGCCCGAGATCGCCACCA |
| | | | GGCCTAAAGTGCGGGATAGA |
| | | | GAAGGCCGGATGAACTACTA |
| | | | CTGGACCCTGGTGGAACCCG |
| | | | GCGACAAGATCACATTTGAG |
| | | | GCCACCGGCAACCTGGTGGT |
| | | | CCCCAGATACGCCTTCACCA |
| | | | TGGAAAGAAATGCCGGCAGC |
| | | | GGCATCATCATCAGCGACAC |
| | | | CCCTGTGCACGACTGCAACA |
| | | | CCACTTGTCAGACAGCCGAG |
| | | | GGCGCCATCAATACCAGCCT |
| | | | GCCTTTCCAGAACGTGCACC |
| | | | CCGTGACCATCGGCAAGTGC |
| | | | CCCAAATACGTGAAGTCCAC |
| | | | CAAGCTGAGACTGGCCACAG |
| | | | GCCTGAGAAACGTGCCCTCC |
| | | | ATTCAGAGCAGAGGCCTGTT |
| | | | TGGAGCCATTGCCGGCTTTA |
| | | | TCGAAGGCGGCTGGACAGGC |
| | | | ATGGTGGACGGATGGTACGG |
| | | | ATACCACCACCAGAACGAGC |
| | | | AAGGCTCTGGCTATGCCGCC |
| | | | GACCTGAAGTCTACCCAGAA |
| | | | TGCCATCGATAAGATCACCA |
| | | | ACAAAGTGAACAGCGTGATC |
| | | | GAGAAGATGAACACCCAGTT |
| | | | CACCGCCGTGGGAAAAGAGT |
| | | | TCAACCACCTGGAAAAGCGC |
| | | | ATCGAGAACCTGAACAAGAA |
| | | | GGTGGACGACGGCTTCCTGG |
| | | | ACATCTGGACCTACAATGCC |
| | | | GAACTGCTGGTGCTCCTGGA |
| | | | AAACGAGAGAACCCTGGACT |
| | | | ACCACGACAGCAACGTGAAG |
| | | | AACCTGTACGAGAAAGTGCG |
| | | | CAACCAGCTGAAGAACAACG |
| | | | CCAAAGAGATCGGCAACGGC |
| | | | TGCTTCGAGTTCTACCACAA |
| | | | GTGCGACAATACCTGCATGG |
| | | | AAAGCGTGAAAAACGGCACC |
| | | | TACGACTACCCTAAGTACAG |
| | | | CGAGGAAGCCAAACTGAACC |
| | | | GCGAGAAGATCGACGGCTCC |
| | | | GGATATATTCCCGAGGCTCC |
| | | | CAGAGATGGCCAGGCCTATG |
| | | | TTCGGAAGGATGGCGAGTGG |
| | | | GTCCTGCTGAGCACATTCCT |
| | | | TGGATCCGGCCTGAACGACA |
| | | | TCTTCGAGGCCCAGAAAATC |
| | | | GAGTGGCACGAGGGCCACCA |
| | | | CCATCATCACCATTGA |

Amino acids from the native sequence that are modified are underlined followed by the replacement amino acid indicated in brackets.

### EXAMPLES

### Example 1: Design of Recombinant Antigen Constructs, Protein Production, Purification and Tetramerization

### Summary

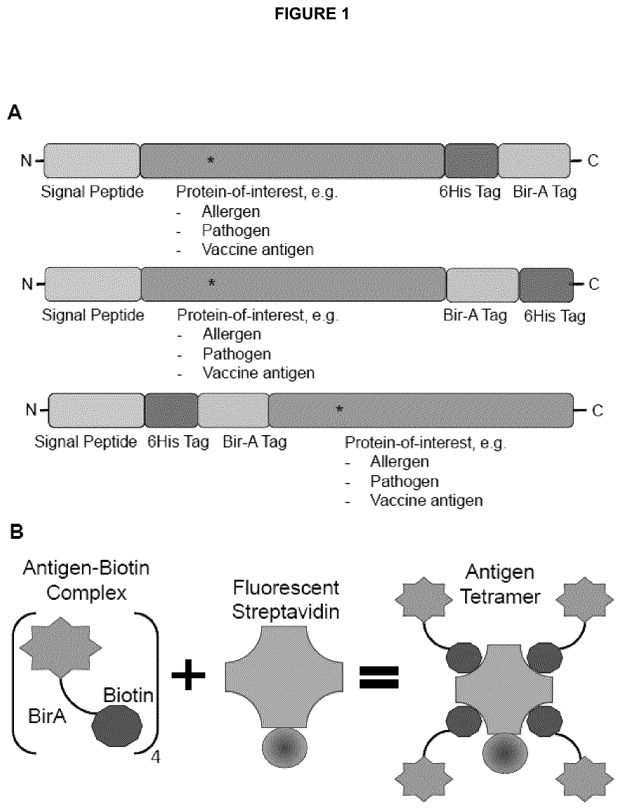

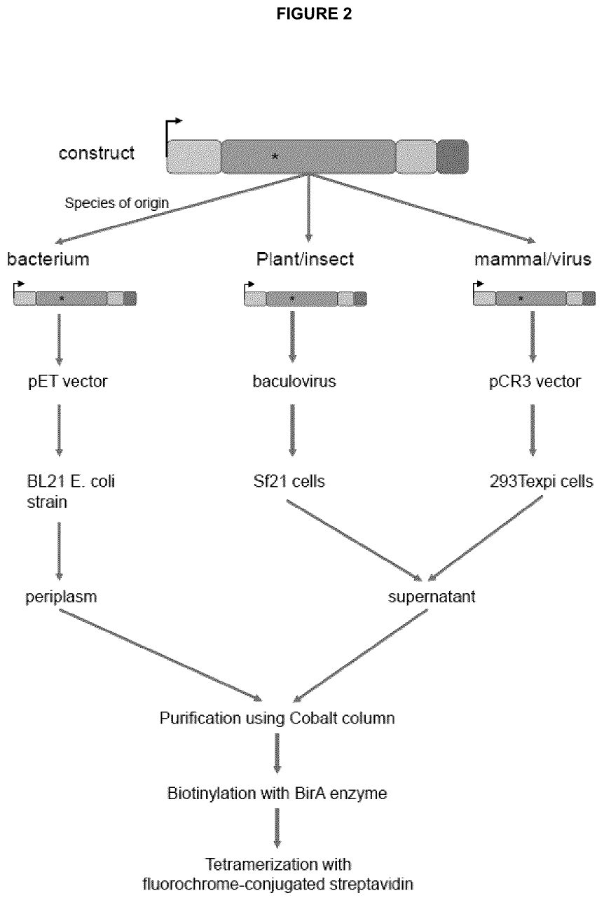

The inventors have used recombinant proteins to introduce modifications that inhibit the enzymatic activity and remove toxicity of the antigens tested. Furthermore, peptide sequences were added to enable efficient purification (6-His tag) and targeted biotinylation (FIG. 1). The protein production pipeline includes steps to generate proteins in the most relevant cell expression system and to have these secreted to ensure that the protein structure and post-translational modifications are as similar to the native protein as possible (FIG. 2).

The recombinant proteins produced by the method described herein were demonstrated to be immunogenic. The recombinant HA proteins from the influenza strains A/Michigan/200/2019 (H1N1) and B/Phuket/3073/2013 (Type B) were recognised by IgG in serum of vaccinated subjects. Further, the recombinant phospholipase A2 (Api m 1) and ryegrass allergen (Lol p 1) were capable of inducing IgE-mediated basophil activation in allergen-sensitized subjects.

Staining of immune cells was achieved by biotinylation of the recombinant proteins and tetramerization using fluorescently labelled streptavidin. The advantage of this system is that it utilises targeted biotinylation which has little to no effect on the rest of the protein and tetramers should increase the avidity for binding Ig (either bound to the surface of cells or expressed on the cell surface). Indeed, all antigen tetramers tested show a high fluorescence signal and antigen-binding cells were clearly distinguished from the non-binding cells by flow cytometry.

### Materials and Methods

Design of recombinant antigen constructs

For the robust detection of cells expressing or binding allergen-specific or antigen-specific immunoglobulins (Ig), we generated the following set of criteria for the design and production of recombinant antigens or allergens:
1. The protein should be non-pathogenic, non-toxic and not enzymatically active to minimize risks for researchers and to prevent cell death in target assays;
2. The protein should be naturally folded and (if applicable) include post-translational modifications to ensure conformational epitopes are present;
3. The protein should be purified without affecting its protein structure; and
4. The protein should contain a tag to facilitate conjugation to allow for multimerization and/or (fluorescent) detection (FIG. 1).

To measure vaccine responses to tetanus (Clostridium tetani) and diphtheria (Corynebacterium diphtheriae), recombinant forms of the toxins were produced with modifications to prevent toxicity. For tetanus toxin, the 451 amino acid C-term domain of the heavy chain (TTC) was produced (as described by Fairweather N F et al. (1986) J Bacteriol 165(1):21-7) and for diphtheria toxin, a single amino acid substitution (G52E) was introduced to generate the non-toxic CRM197 variant (as described by Giannini G et al. (1984) Nucleic acids Res 12(10):4063-9 and Malito E et al. (2012) Proc Natl Acad Sci USA, 109(14):5229-34). Both constructs included a leader sequence for targeting production to the periplasm of E. coli (MIKFLSALILLLVTTAAQA) (as described by Goffin P et al. (2017) Biotechnol J, 12(7)). In addition, both constructs contained a C-terminal 6 histidine (6-His) and a 15 residue BirA Substrate Peptide (BSP) (LHHILDAQKMVWNHR) (as described by Schatz P J (1993) Biotechnology (NY), 11(10):1138-43) The constructs were codon optimized for E. coli and were cloned into the pET30 vector (Novagen).

To measure vaccine responses to the trivalent and tetravalent influenza vaccines, recombinant Haemagglutinin (HA) antigens from the A/Michigan/45/2015 (AM15), A/Brisbane/02/2018 (AB18) and B/Phuket/3073/2013 (BP13) strains were generated. All constructs contained the C-terminal AviTag (GLNDIFEAQKIEWHE) BirA target sequence and a 6-His tag (as described in Whittle J R et al. (2014) J Virol, (8):4047-57, Wheatley A K et al. (2016), Sci Resp 25; 6:26478 and Liu Y et al. (2019) Nat Commun, 18;10(1):324). The constructs contained their native leader sequences for secretion. To prevent a-specific binding to sialic acids on cells, point mutations were introduced in AM15 and AB18 (Y98F) and PB13 (T139G). The constructs were codon optimized for Homo sapiens and cloned into the pCR3 vector (invitrogen) for expression in 293Expi cells.

To measure allergic sensitization to insect venom, aeroallergens and food allergens venom, major allergen components were recombinantly produced from honey beevenom (Api m 1) ryegrass pollen Lol p 1 and Lol p5), Timothy grass pollen (Phl p 1), house dust mite (Der p 2), cat dander (Fel d 1), peanut (Ara h 2) and black tiger shrimp (Pen m 1). The sequences were obtained from the WHO/IUIS Allergen Nomenclature database (http://www.allergen.org/) and nucleotide sequences can be found in GenBank: X16709.1 for Api m 1, and M57474.1 for Lol p 1).

All constructs with the exception of Fel d 1 contained the Api m 1 N-terminal leader sequence for extracellular production and either C-terminal or N-terminal AviTag and 6-His sequences. Enzymatic activity was prevented through the introduction of point mutations: H34Q for Api m 1, (as described by Forster E et al. (1995) J Allergy Clin Immunol, 95(6):1229-35) and H104V for Lol p 1 and Phl p 1 (as described by Grobe K et al. (2002) Eur J Biochem 269(8):2083-92). All constructs with the exception of Fel d 1 were codon optimized for Spodoptera frugiperda (fall armyworm) and cloned into the pFastBac vector (Thermo Fisher Scientific), prior to incorporation into a Bacmid for baculovirus production. Fel d 1 was produced with its native leader sequence and C-terminal AviTag and 6His tags, codon optimized for HOMO sapiens and cloned into the pCR3 vector (Invitrogen) for expression in 293Expi cells (as for influenza constructs).

### Protein Production, Purification and Tetramerization

Bacterial constructs were inserted into BL21(DE3) pLysE E. coli cells by heat shock, and single colonies were cultured overnight (16 hrs) at 37° C. in 10 ml Luria-Bertani (LB) broth with Kanamycin and Chloramphenicol antibiotics. These 10 ml cultures were then transferred into 1 L culture and grown to OD 600 of 0.6-0.7 at 37° C. for 4-7 hrs. Protein production was then induced by addition of 1 mM IPTG, and bacteria were cultured at 27° C. overnight (16 hrs). Bacterial pellets were collected, and periplasmic fractions were prepared by osmotic shock at 4° C. First, pellets were thoroughly resuspended in a 30 mM Tris-HCl, 20% sucrose solution at pH 8 for 10 mins. Subsequently, cells were pelleted and resuspended in 5 mM MgSO₄ for 10 mins. Following final pelleting, supernatants were collected for protein purification.

Viral constructs were transfected using Expifectamine into 293Expi cells (Thermo Fischer Scientific) and cultured in suspension in Opti-MEM I Reduced-Serum Medium (Gibco) at 37° C. on a shaker for 5 days. Subsequently, culture supernatants were harvested.

Insect and plant allergens constructs were incorporated into Bacmids encoding baculovirus. Bacmids were transfected into Sf21 cells and cultured at 27° C. Supernatants from infected Sf21 cultures were clarified by centrifugation.

As all protein constructs contained a 6-His tag, these were purified from bacterial periplasm preparations, and 293Expi and Sf21 culture supernatants through retention on a cobalt column. Supernatants were gravity-fed through a 25 ml column packed with 4 ml Talon NTA-cobalt-agarose beads (Clontech). Beads were washed with PBS and proteins were eluted with PBS, pH 8.5, containing 200 mM imidazole. Eluate was dialyzed against 10 mM TRIS, pH 7.5.

All recombinant proteins contained a BSP tag for biotinylation, and were biotinylated by an overnight incubation at RT with BirA enzyme 2.5 μg/ml in 10 mM TRIS containing Bicine-HCl 62.5 mM, ATP 12.5 mM, 12.5 mM MgOAc, 62.5 μM D-biotin. Proteins were dialyzed against PBS. To tetramerize our antigens, we first determined the required volume of streptavidin by calculating a 4:1 molar ratio of antigen:streptavidin. Antigens were tetramerized by adding 1/10th the volume of Streptavidin-fluorophore conjugates (PE, APC, BUV395, BUV737, all from BD Biosciences) stepwise every 5 mins, repeated 10× at room temperature.

Western Blotting Baculovirus-containing Sf21 supernatant, 293T Expi cell supernatant and E coli periplasm preps were mixed with 6× reducing or non-reducing buffer (0.1 M Tris-HCl (pH 6.8), 0.2% bromophenol blue, and 20% glycerol, reducing buffer includes 4% SDS and 50 mM DTT). Reduced samples were heated at 85° C. for 10 mins. Protein samples were loaded onto 4-15% Mini-PROTEAN® TGX Stain-Free gels (Bio-Rad) and separated at 200V for 30 mins. Proteins were transferred onto PVDF membranes (Bio-Rad) using the Trans-Blot Turbo Transfer System (Bio-Rad). Membranes were probed with mouse anti-His (clone: 27471001, category #18594, GE Healthcare) followed by goat anti-mouse IgG HRP (clone: NA9310N, category no. #D614011, Cell Signaling Technology). PVDF membranes were developed using Amersham ECL Western Blotting Detection Reagent (GE Healthcare Life Sciences) and chemiluminescence was detected using a ChemiDoc Imager (Bio-Rad).

### Enzyme-Linked Immunosorbent Assay (ELISA)

ELISA plate wells were coated with recombinant proteins, blocked with 2% bovine serum albumin in PBS (Sigma Aldrich, Darmstadt, Germany) and incubated with serial dilutions of serum samples. Bound IgE was detected using rabbit polyclonal anti-hIgE (Dako) followed by goat anti-rabbit IgG HRP (Bio-Rad). ELISA were developed using TMB (Thermo Scientific) and the reaction stopped with 1 M HCl. Absorbance (OD 450 nm) was measured using a FLUOstar Optima plate reader (BMG Labtech).

### Study Participants

Adult healthy controls were enrolled in a low-risk reference value study (Monash University project 2016-0289), and consented to collection of basic demographics (age, sex, history of immunological and hematological disease) and donation of 40 ml blood. From those who had indicated to get the 2019 influenza vaccine of their own volition, a second blood sample was taken 4 weeks after vaccination.

### Results

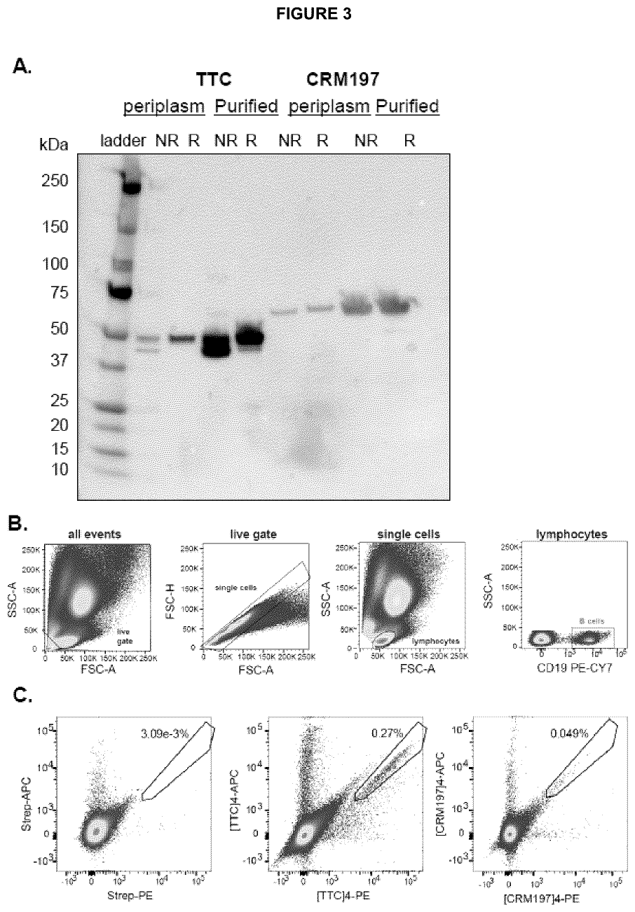

Recombinant antigens have been generated with BSP and 6-His peptide tags as described in the Methods section. Recombinant, non-toxic tetanus toxin (TTC) and diphtheria toxin (CRM197) constructs were generated in E. coli and purified from periplasm, prior to purification in a cobalt column. Western blotting with an anti-His antibody (FIG. 3) showed the presence of protein in periplasm preps and enrichment following purification. When run under non-reduced conditions, 2 bands were seen for TTC around 45 and 50 kDa, whereas only a 50 kDa band was seen under reduced conditions. Thus, it appears that there is one product with potentially two altered conformations. The product was slightly shorter than predicted based on sequence (57 kDa), suggestive of cleavage of the leader peptide. For CRM197 only one band was seen under both non-reduced and reduced conditions of −60 kDa, and this was also slightly shorter than predicted based on sequence only (64 kDa).

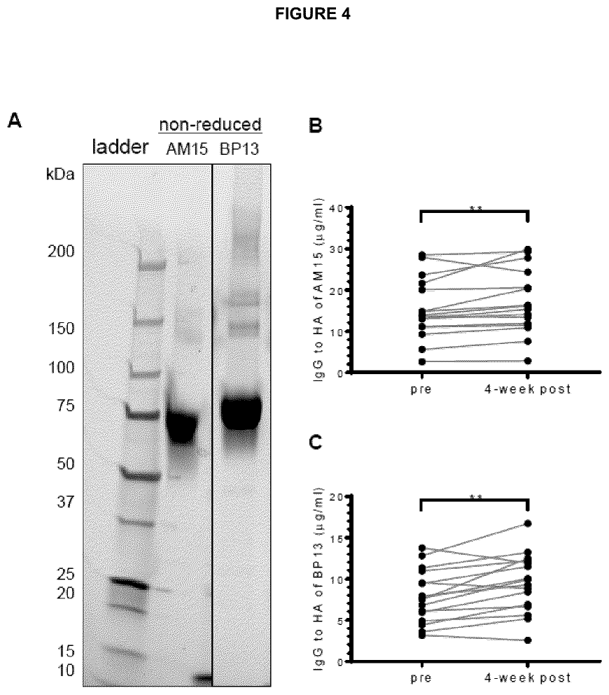

HA proteins from one type A (AM15, A/Michigan/45/2015) and one type B (BP13, B/Phuket/3073/2013) influenza strain were produced with AviTag and 6-His peptide tags in 293Texpi cells, and purified from supernatant on a cobalt column. Western blotting using an anti-His antibody under non-reduced conditions showed single bands of −65 kDa (AM15) and −75 kDa (BP13) (FIG. 4A). These were close to the predicted size of 64 kDa for both, indicating that the proteins were produced as monomers.

To examine whether the products were specifically recognized by IgG in serum of vaccinated individuals, we determined the levels of HA-specific IgG to each of the HA proteins in 16 individuals pre- and post-vaccination with the 2019 quadrivalent seasonal influenza vaccine (Afluria-Quad (Seqirus), Influvac tetra (Mylan Health) or Fluquadri (Sanofi-Aventis)) who had been vaccinated previously in 2018. All individuals had detectable levels of IgG against HA from AM15 (range, 2.6-29 μg/ml) and BP13 (range, 3.1-14 μg/ml) prior to the 2019 vaccination, and these specific IgG levels were significantly higher 4 weeks post booster vaccination (both p<0.01; paired t test, FIGS. 4B and 4C).

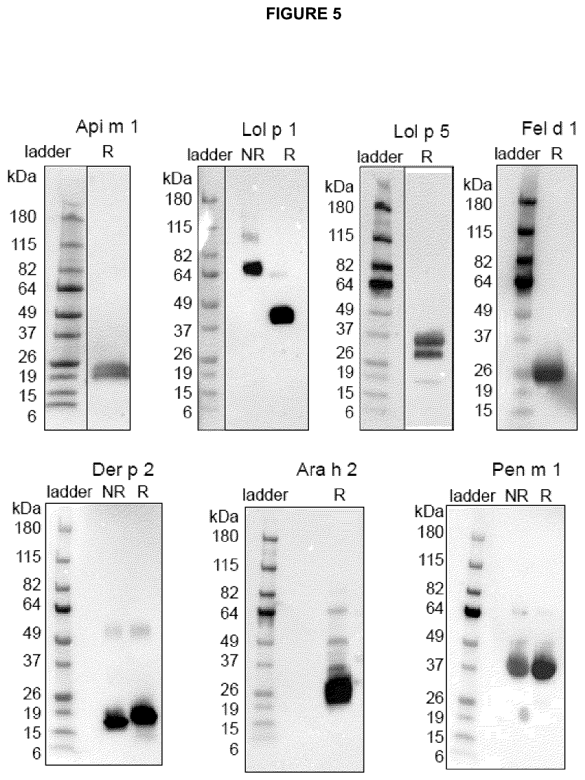

Seven major allergens from bee venom (Api m 1) ryegrass (Lol p 1, Lol p 5), Timothy grass pollen (Phl p 1), house dust mite (Der p 2), peanut (Ara h 2) and black tiger shrimp (Pen m 1) were produced in Sf21 insect cells using the baculovirus expression system. The major cat dander allergen, Fel d 1, was produced in 293Texpi cells. The constructs contained AviTag and 6-His peptide tags, and were purified from culture supernatant using cobalt columns. The recombinant proteins were evaluated by Western blotting using an anti-His antibody and showed bands at the expected sizes, or slightly larger due to post-translational modifications (e.g. glycosylation) (FIG. 5). For Lol p 1 a single non-reduced product of ˜70 kDa and a single reduced product of −42 kDa were observed. This suggests that Lol p 1 is produced as a dimer, which would correspond well with the prediction of its native configuration based on the structure of the highly homologous Phl p 1 allergen (Protein database entry: 1N10 (Flicker S et al. (2006) J Allergy Clin Immunol 117(6):1336-43).

### Example 2: Detection of Allergen Sensitisation Via Staining and Flow Cytometric Detection of Basophils

### SUMMARY

The inventors have demonstrated the ability to detect antigen-specific Ig on soluble Ig binding cells (e.g. basophils). The inventors demonstrate that staining of basophils is very specific for the allergen-sensitized subjects as the signal for allergen was over 2-fold higher than streptavidin alone. None of the non-sensitised controls showed any signal over 1.6-fold. Therefore, allergen-straining can be utilised for laboratory testing of allergen sensitisation. Importantly, as the method utilises multicolour flow cytometry, the test can easily be multiplexed with several allergens being conjugated to distinct fluorescent fluorophores allowing several allergens to be tested within a single tube.

As only 2-3 fluorescent channels are required to detect basophils, there are plenty of available options for fluorescent conjugates depending on the flow cytometer used. For example, 5 on a 3-laser, 8-parameter FACS Canto or up to 14 parameters on a 5-laser LSRFortessa X-20, etc. Fluorescently-labelled streptavidins are readily available and can be easily implemented to be conjugated to the recombinant proteins (PE, APC, BUV395 and BUV737).

Basophil staining has a number of advantages over the standard basophil activation tests (BAT) as it provides the opportunity to perform component-resolved diagnostics (CDR) in a single sample tube, whereas BAT can only be read out for a single allergen (mix) per tube. Thus, allergen-tetramers with distinct fluorochromes could be combined in a single staining cocktail to dissect allergen-sensitization and cross-reactivity.

### Materials and Methods

Study participants

Patients with bee venom, ryegrass allergy, Timothy grass, house dust mite, cat dander, peanut and/or shrimp were recruited from the Alfred Health Allergy clinic after informed consent was obtained for blood collection and medical information (Alfred Health, ethics projects #514/13 and #509/11). Allergen sensitization was defined as being positive for a prick test and/or RAST. Patients made either one or two donations of 40 ml blood, one prior to the start of allergen-immunotherapy and where applicable one after 2-4 weeks (bee venom allergy) or 4 months (ryegrass allergy) of allergen-immunotherapy.

The study was carried out according to the principles of the Declaration of Helsinki, and was approved by local human research ethics committees.

Flow cytometry of basophils

Absolute numbers of leukocytes were determined using a lyse-no-wash method within 24 hrs of blood sampling in Vacutainers containing EDTA (BD Biosciences). 50 μl whole blood was added to a TruCount tube (BD Biosciences) together with an antibody cocktail of 20 μl to stain CD3, CD4, CD8, CD16, CD45 and CD56. Following incubation for 15 mins at room temperature, 500 μl, 0.155 M NH₄Cl was added to lyse red blood cells for 15 mins. Subsequently, the mixture was stored in the dark at 4° C. prior to acquisition on a flow cytometer within 2 hrs.

To identify circulating basophils, whole blood was incubated with anti-CD123-BV605 (6H6; BioLegend) and anti-IgE-FITC (goat anti-human; Thermo Fisher). Red blood cells were lyzed with ammonium chloride solution (NH₄Cl 154 mM, KHCO₃ 10 mM, EDTA 1 mM) and washed. To identify live cells, remaining cells were resuspended in wash buffer containing 7AAD (BD Biosciences). Samples were stored in the dark at 4° C. before analysis by flow cytometry within 2 hrs.

### Basophil Activation

To prime circulating basophils, whole blood was incubated for 10 mins at 37° C. in stimulation buffer (Hepes 20 mM, NaCl 133 mM, KCl 5 mM, CaCl₂) 7 mM, CaCl₂) 3.5 mM, BSA 1 mg/ml, rIL-3 2 ng/ml, Heparin 20 μl/ml, pH 7.4). Basophils were activated with Streptavidin-fluorophore conjugates (0.5 μg/ml) or allergen tetramers (1 μg/ml) for 20 mins at 37° C. Activation was stopped by incubating on ice for 5 mins. Serum was removed by washing with cold wash buffer (Hepes 20 mM, NaCL 133 mM, KCl 5 mM, EDTA 0.27 mM, pH 7.3). Basophil activation was examined by flow cytometry using markers described above to defined basophils (anti-CD123, anti-IgE) and positivity for surface CD63 using anti-CD63-PE (H5C6; BD Biosciences).

### Flow Cytometer Setup

All flow cytometry was performed across 3 instruments in our flow core facility that contained either 4 lasers (BD LSRII and BD LSRFortessa) or 5 lasers (BD LSRFortessa X-20) with a nearly identical set-up for the shared 4 lasers. Instrument set-up and calibration were performed using standardized EuroFlow SOPs (as described in Kalina T et al. (2012) Leukemia 26(9):1986-2010) with in-house optimization for the additional three fluorescent channels (V610, V710 and YG610) (as described in Edwards ESJ et al (2019), Front Immunol. 10:2593).

### Data Analysis and Statistics

All data were analyzed with FACS DIVA v8.0.1 (BD Biosciences) and FlowJo v10 software packages (FlowJo, LLC). Statistical analysis was performed with the non-parametric Mann-Whitney U test. Statistical analysis of sampling distributions was assessed with the Chi-square test. For all tests, p<0.05 were considered significant.

### Results

### Initial Testing of Antigens and Allergens

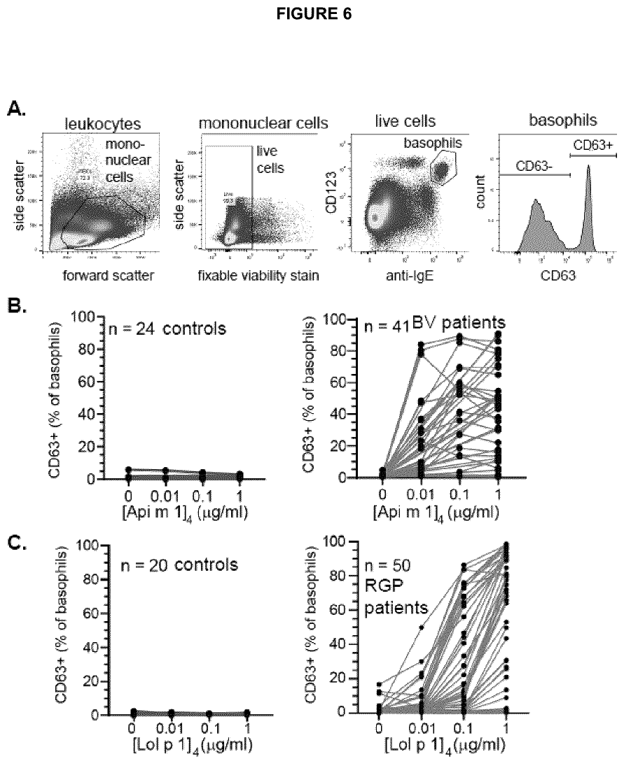

To examine whether the recombinant allergens are recognized by IgE and can mediate allergic responses, we determined their capacity to activate basophils using the flow cytometric basophil activation test (BAT) (as described in Hemmings O et al. (2018) Curr Allergy Asthma Rep, 18(12):77). Following in vitro activation of basophils in whole blood with either streptavidin-APC, allergen-streptavidin-APC, or allergen extracts, basophils were processed and analyzed via flow cytometry. The samples were analyzed by electronically gating on high expression of CD123 and IgE (FIG. 6A). Activated basophils were defined as being positive for surface expression of CD63 (as described in Hemmings 0 et al. (2018) Cuff Allergy Asthma Rep 18(12):77).

Fresh whole blood samples of 20 subjects with bee venom sensitization and 24 non-allergic controls were tested using the BAT test. For all samples tested the streptavidin-APC alone (negative control) did not result in CD63 surface expression (FIG. 6B). Basophils from all patients were activated by the Api m 1 tetramers, whereas none of the controls were. Similarly, blood basophils of 50 subjects with ryegrass pollen sensitization and 20 controls were stimulated with streptavidin-APC, and Lol p 1 tetramers (FIG. 6C). For all subjects, the streptavidin-APC alone hardly resulted in CD63 expression however, the Lol p 1 tetramers yielded higher frequencies of CD63+ basophils in patients but not in controls.

Together, these results illustrate that our recombinant antigen production pipeline has the capacity to produce proteins that are recognized by IgG and IgE antibodies, and are immunologically active.

### Detection of Allergen Sensitization Via Fluorescent Antigen Staining of Basophils

The BAT assay works on the main principle that basophils in blood bind high levels of soluble IgE through high-affinity FcεRI on their surface. These soluble IgE have diverse specificities for antigen, and in the case of an allergen-sensitized patient, the IgE molecules on the surface of a basophil include those with a specificity to that particular allergen (as reviewed in Hoffmann H J et al. (2015), Allergy 70(11):1393-405). Thus, upon in vitro incubation, these IgE will bind to allergen, and if multiple molecules will bind, these will cross-link and activate the basophile through FcεRI. Despite this being a sensitive laboratory test, there are certain limitations:
1. Careful handling of blood as storage for >4 hrs and under suboptimal conditions can render the basophils unresponsive;
2. A single BAT only produces read out to the response of the whole allergen extract, so no information on specific allergen sensitization can be discerned, and this would require an additional test for each specific allergen.

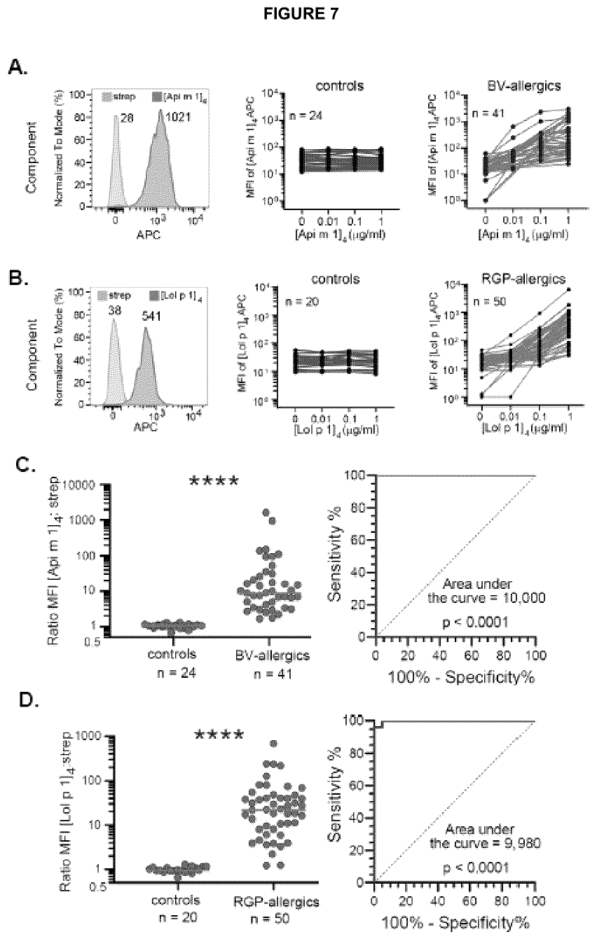

The specificity for basophil activation is based on the presence of allergen-specific IgE bound to its FcεRI. Thus, we determined if our recombinant allergens (Api m 1 and Lol p 1), upon tetramerization with an allophycocyanin (APC)-labeled streptavidin, could bind specifically to basophils of allergen-sensitized individuals. Whole blood of non-sensitized controls and allergen-sensitized individuals was incubated with an antibody cocktail and either streptavidin-APC or allergen-streptavidin-APC conjugates. Following flow cytometric gating of CD123+ IgE+ basophils (FIG. 6A), the fluorescence intensity of the APC signal was determined in these cells (FIGS. 7A and 7B). In non-sensitized controls, the streptavidin-APC, (Api m 1)₄-APC and (Lol p 1)₄-APC median fluorescent intensities were very similar around 10-60 with the allergen-tetramer signals typically being 1.1 to 1.5-fold higher than the streptavidin-APC signals (FIGS. 7C and 7D). In contrast, the fluorescent intensities of the allergen tetramers were much higher on basophils from allergen-sensitized patients. All fluorescent signals were higher for the Api m 1 in bee venom allergic patients and for nearly all ryegrass pollen allergic patients using Lol p 1. The signal intensities were 2 to 1000-fold higher than the streptavidin-APC signals on basophils of the same patient. Thus, the allergen-tetramers specifically stain basophils of sensitized patient. Importantly, sensitization can be determined by the signal intensity or by the fold difference over streptavidin staining only. This is illustrated by receiver operator characteristics (ROC) curves with extremely high areas under the curve (AUC) of 10,000 for Api m 1 and 9980 for Lol p 1 (FIGS. 7C and 7D) for separating sensitized from unsensitized individuals.

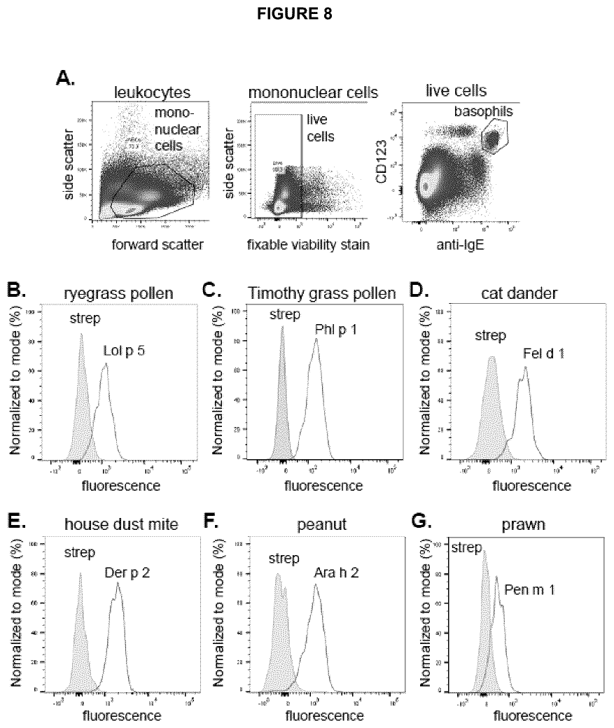

Similar to Api m 1 for bee venom and to Lol p 1 for ryegrass pollen, recombinant allergen tetramers of the other 6 allergens were generated (Lol p 5, Phl p 1, Der p 2, Fel d 1, Ara h 2, Pen m 1). Following incubation of basophils from patients with relevant allergen sensitization (FIG. 8A), all 6 allergen tetramers specifically stained basophils with 2-10 fold increase in signal intensity over streptavidin only (FIG. 8B-G).

### Example 3: Assessment of Allergen Desensitisation Via Staining and Flow Cytometric Detection of B Cells

### SUMMARY

The inventors have demonstrated the ability to detect antigen-specific surface Ig expressing cells (B cells).

B cell staining with fluorescent antigens can be multiplexed. Detection of antigen-specific B cells can be used to assess humoral immune responses in multiple situations. In the case of allergies, it can complement the findings of allergen sensitization as observed by basophils staining.

The present invention allows for changes in the B cell compartment to be monitored over time following treatment with allergen immunotherapy (AIT). This can be measured via a shift to more IgG-expressing B cells, especially IgG2 and IgG4 expressing B cells, as a measure for successful desensitisation.

In addition to allergies, responses to infections and vaccination can be monitored in the B cell compartment. Similarly, changes in the Ig isotype and IgG subclass usage could reflect successful primary or booster responses. Moreover, measurements of antigen-specific B cells could provide a means to examine vaccination responses in individuals receiving Ig-replacement therapy (IgRT), as in these individuals, serum IgG measurements do not reflect the host response but the donor IgG composition.

### Materials and Methods

Flow cytometry of B cells

To immunophenotype circulating allergen-specific B cells, peripheral blood mononuclear cells (PBMCs) were incubated with both the PE-conjugated variant and the APC-conjugated variant of the same allergen tetramer. In addition, an antibody cocktail was added that included CD3-BV711 (UCHT1), CD19-PE-Cy7 (SJ25C1), CD27-BV421 (M-T271), anti-IgD-PE-CF594 (IA6-2), anti-IgG-BV786 (G18-145; all from BD Biosciences), CD38-APC-Cy7 (HIT2), CD123-BV605 (6H6), anti-IgM-BV510 (MHM088, all from BioLegend), anti-IgE-FITC (goat anti-human; Thermo Fisher) and Fixable Viability Stain 700 (BD Biosciences). Following incubation for 15 mins at room temperature, PBMCs were washed with PBS containing 0.2% BSA. Samples were stored in the dark at 4° C. prior to data acquisition on a flow cytometer within 2 hrs.

To immunophenotype circulating influenza HA-specific B cells, PBMCs were incubated with both the BUV395-conjugated variant and the BUV737-conjugated variant of the same HA antigen tetramer. In addition, an antibody cocktail was added that included anti-CD3-BV711 (UCHT1), anti-CD19-PE-Cy7 (SJ25C1), anti-CD21-BV711 (B-ly4), anti-CD27-BV421 (M-T271; all from BD Biosciences), anti-CD38-APC-Cy7 (HIT2), anti-IgD-PerCP-Cy5.5 (IA6-2), anti-IgM-BV510 (MHM088, all from BioLegend), anti-IgA-PE-Vio615 (REA1014, Miltenyi Biotec), anti-IgG1-PE (SAG1), anti-IgG2-FITC (SAG2), anti-IgG2-PE (SAG2), anti-IgG3-FITC (SAG3), anti-IgG4-APC (SAG4; all from Cytognos) and Fixable Viability Stain 700 (BD Biosciences). Following incubation for 15 mins at room temperature, PBMCs were washed with PBS containing 0.2% BSA. Samples were stored in the dark at 4° C. prior to data acquisition on a flow cytometer within 2 hrs.

### Flow Cytometer Setup

All flow cytometry was performed across 3 instruments in our flow core facility that contained either 4 lasers (BD LSRII and BD LSRFortessa) or 5 lasers (BD LSRFortessa X-20) with a nearly identical set-up for the shared 4 lasers. Instrument set-up and calibration were performed using standardized EuroFlow SOPs as previously described in (Kalina T et al. (2012) Leukemia, 26(9):1986-2010) with in-house optimization for the additional three fluorescent channels (V610, V710 and YG610) (as described in Edwards ESJ et al., (2019) Front Immunol. 10:2593).

### Data Analysis and Statistics

All data were analyzed with FACS DIVA v8.0.1 (BD Biosciences) and FlowJo v10 software packages (FlowJo, LLC). Statistical analysis was performed with the non-parametric Mann-Whitney U test. Statistical analysis of sampling distributions was assessed with the Chi-square test. For all tests, p<0.05 were considered significant.

Results

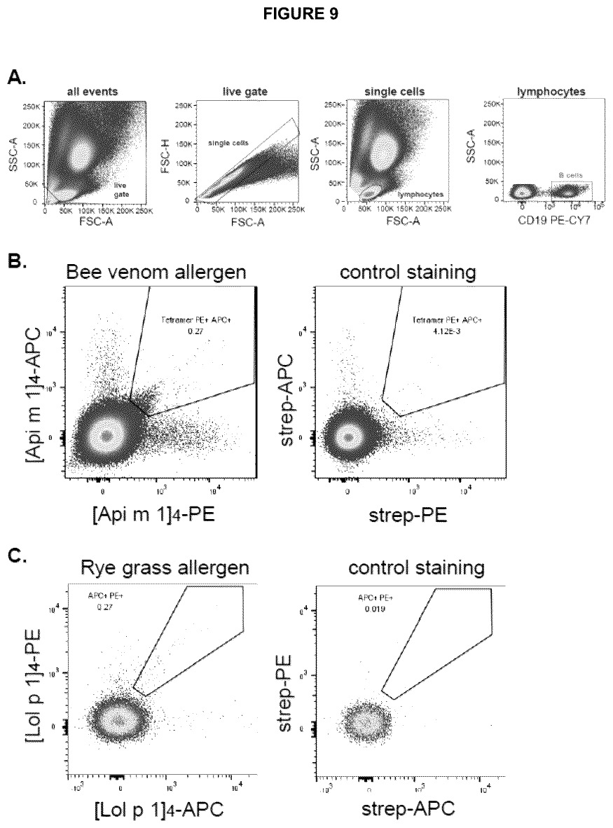

B cells each carry a surface Ig molecule with unique specificity. Theoretically, among the pool of naive B cells, a small fraction will have specificity to foreign proteins, such as allergens and vaccine antigens. Furthermore, in individuals who have been exposed to these proteins, it is to be expected that a memory B cell population exists. Finally, in allergen-sensitized patients, who produce soluble IgE to the allergen, there is a potential fraction of B cells expressing allergen-specific IgE. To test this, we combined ex vivo staining of B cells with an antibody cocktail with our fluorescent antigen tetramers. As fluorophores such as phycoerythrin (PE) and APC are large proteins to which antibodies can be formed, incubation was always performed with two protein conjugates. After electronic gating on single live cells in whole blood, CD19+ B cells were defined (FIG. 9A) and among all B cells, the fraction of antigen-tetramer positive cells was determined. As expected, among B cells of a bee venom sensitized individual, there were PE-specific cells, as well as APC-specific cells (FIG. 9B). These were also evident in both antigen-tetramer stains and in streptavidin-only stains. However, in the antigen-tetramer stain, there was a small fraction of 0.27% that was double-positive for the Api m 1 tetramers. This fraction was almost completely absent in the streptavidin-only stains. Similarly, within total B cells of a patient with ryegrass pollen allergy, a fraction of Lol p 1 was clearly identified. Thus, the Api m 1 and Lol p 1 allergen-tetramers can be utilized to specifically stain allergen-specific B cells.

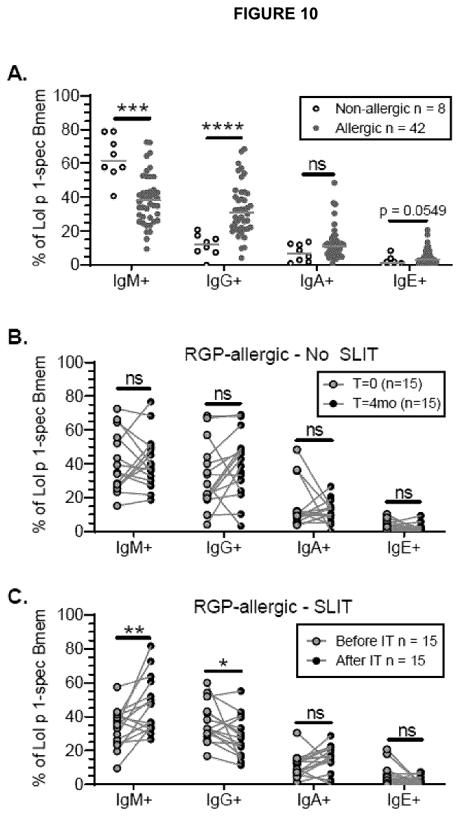

Examination of the Lol p 1 specific memory B cells revealed that ryegrass allergic patients had increased frequencies of IgG+ Bmem as compared to non-allergic controls (FIG. 10A). This was at the expense of IgM+ Bmem. Repeat sampling after 4 months revealed no change in patients out of the pollen season under standard treatment (FIG. 10B), whereas patients that had undergone 4 months of sublingual immunotherapy (SLIT) had significantly higher fractions of IgM+ Bmem and lower IgG+ Bmem. Thus, SLIT is associated with changes in the allergen-specific Bmem compartment towards a profile similar to that seen in unsensitized controls (FIG. 10A).

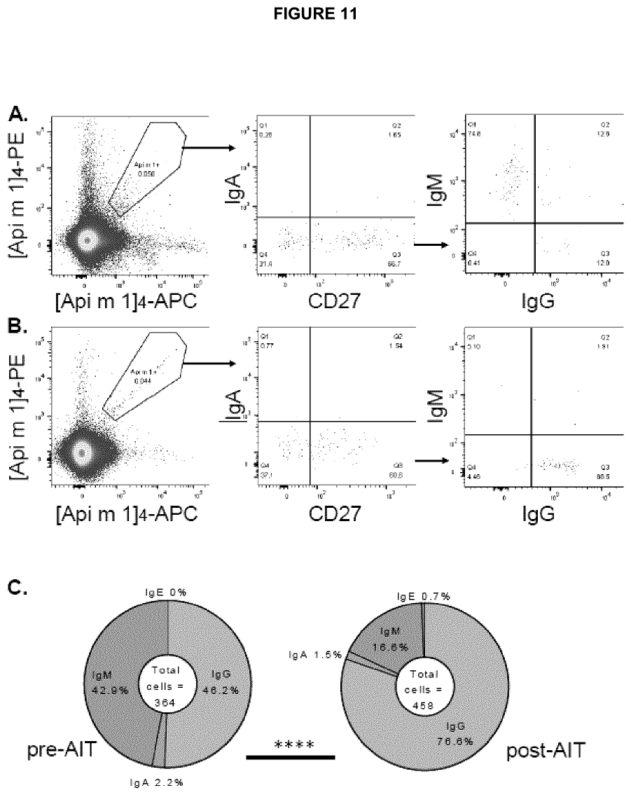

Using a more extensive antibody cocktail, we were able to further immunophenotype Api m 1-specific B cells in bee venom sensitized individuals before and 2-4 weeks after start of ultra-rush allergen-immunotherapy (AIT). Both pre- and post-AIT, the Api m 1-specific fraction consisted of both naive (CD27-IgM+) and memory (CD27+) B cells. Importantly, within the fraction of memory cells, there appeared to be a shift from predominant IgM expression to IgG expression (FIG. 11). This could indicate repeated immune responses due to repeated allergen exposure, and could be a measure for therapy success.

### Example 4: Monitoring of Vaccination Responses

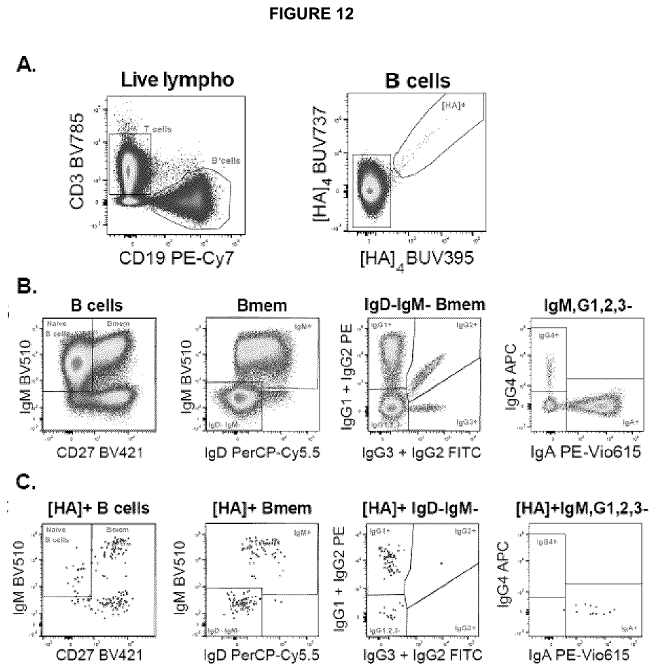

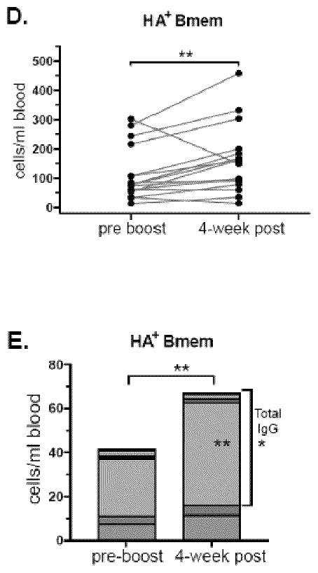

Similar to the recombinant allergen-tetramers, we also performed immunophenotyping of vaccine antigen-specific B cells. B cells from a healthy adult after booster vaccination with the 2019 quadrivalent vaccine were immunophenotyped with an antibody cocktail and two recombinant HA tetramers from AM15 (FIG. 12A). Using either a combination of PE- and APC-conjugates or a combination of BUV395—and BUV737-conjugates, HA-specific B cells were clearly detected at <1% of total B cells. Within the HA-specific B cells, multiple subsets were identified. Specifically, using Ig isotype and IgG subclass antibodies, memory B cells expressing IgG1, IgG2, IgG3, IgG4 and IgA could be separated. Such division might be useful in the context of evaluation of booster vaccinations as repeated exposure to antigens is associated with increased usage of IgG2 at the expense of IgG1 and IgG3.

Immunophenotyping of vaccine-antigen specific B cells is shown in FIG. 12. Stepwise gating of CD3+ T cells and CD19+ B cells (left panel), followed by double discrimination of AM15 specific B cells is shown in FIG. 12A. Subsetting of total B cells to discriminate naïve (IgM+CD27-) from memory B(mem) cells (all others; left panel), followed by separation of IgM/IgD+ unswitched Bmem from IgM-/IgD-switched Bmem, within switched Bmem, IgG1, IgG2, IgG3, IgG4 and IgA expression B cells can be distinguished shown in FIG. 12B. Subsetting of AM15-specific B cells with a similar approach as in B is shown in FIG. 12C. Influenza booster vaccination results in increased numbers of AM15-specific Bmem cells and through detailed immunophenotyping it was assessed that these specifically concerned IgG1+ Bmem as shown in FIG. 12D. Statistics, Mann-Whitney U test; *, p<0.05; **, p<0.01.

### Example 5: Monitoring Immune Response to Viral Infection

To measure immune responses to coronaviruses, recombinant nucleocapsid and spike protein antigens were generated from SARS-CoV and SARS-CoV2. All constructs contained the C-terminal AviTag (GLNDIFEAQKIEWHE) BirA target sequence and a 6-His tag. The constructs contained the Ig leader sequence (MVLSLLYLLTALPGILS) or Fel d 1 leader sequence (MRGALLVLALLVTQALG) for secretion. The wild-type nucleocapsid proteins from SARS-CoV and SARS-CoV2 and a mutant variant from SARS-CoV2 (pos 256-261 KKPROK4GGPROG) to improve protein stability were generated (Table 1). The complete extracellular regions of the spike protein, as well as S1 and S1B domains were generated. The full-length spike protein contained mutations to prevent cleavage between S1 and S2 (pos 682-685 RRAR→SGAS) (Walls et al. (2020) Structure, Function, and Antigenicity of the SARS-CoV-2 Spike Glycoprotein, Cell 181, 281-292) and to improve stability of the S2 domain (986-987 KV→PP) (Table 1).

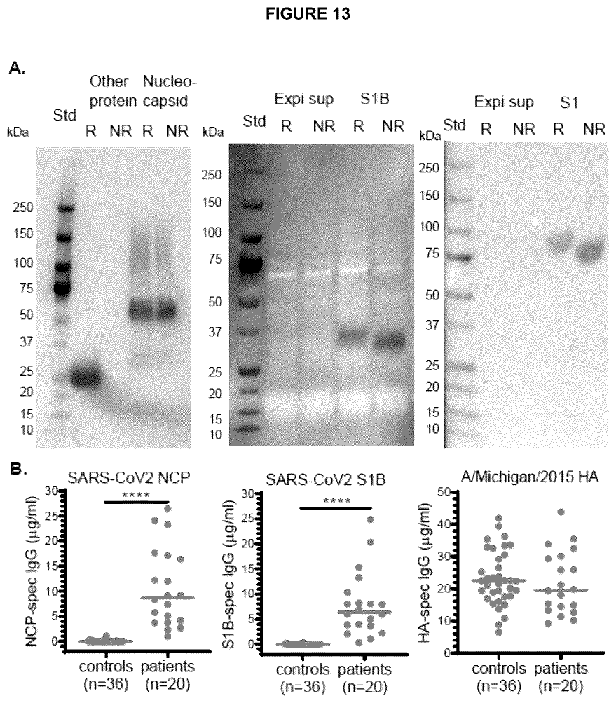

The detection of recombinant nucleocapsid and S1 B proteins from SARS-CoV2 is shown in FIG. 13. As shown in FIG. 13A, western blots using an anti-His detection antibody of nucleocapsid (left panel) and S1B (right panel). Loaded are culture supernatants of sf21 cells after enrichment for 6His-tag containing proteins on a cobalt-loaded retention column. Calculated molecular weights of reduced proteins are −48 kDa (N) and −30 kDa (S1B). Abbreviations: NR, non-reduced; R, reduced. FIG. 13B shows serum IgG specific to antigens as determined by ELISA using the antigen to capture. Thirty-six historic samples (sampled in 2019 and Q1 2020) of previously influenza-vaccinated healthy adults were run, as well as serum samples from 4-20 individuals after with COVID-19 or after recovery (convalescent). Statistics, Mann-Whitney U test.

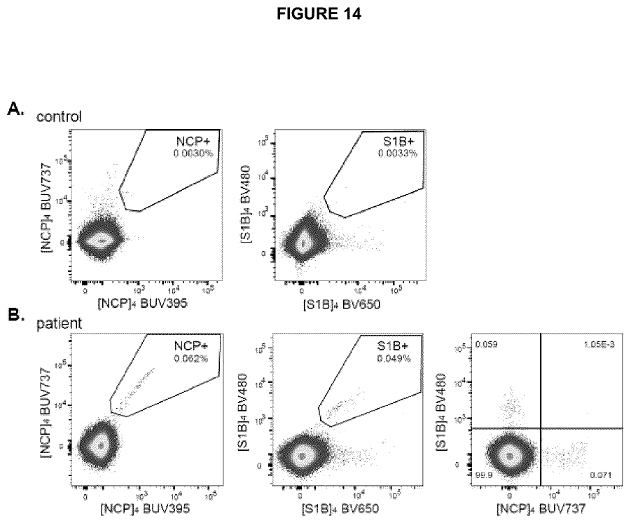

The detection of B-cells with surface Ig specific for SARS-CoV2 nucleocapsid protein in a patient after recovery from COVID-19 is shown in FIG. 14. Specifically, FIG. 14A shows 2D plots showing B cells from an uninfected control stained with BUV395-conjugated and BUV737-conjugated nucleocapsid tetramers (left plot) and BV480-conjugated and BV650-conjugated S1 B tetramers. FIG. 14B shows 2D plots showing B cells from COVID-19 convalescent patients stained with the same NCP and S1 B tetramers, showing well-defined populations for both (left and middle plot), which were mutually exclusive (right plot).

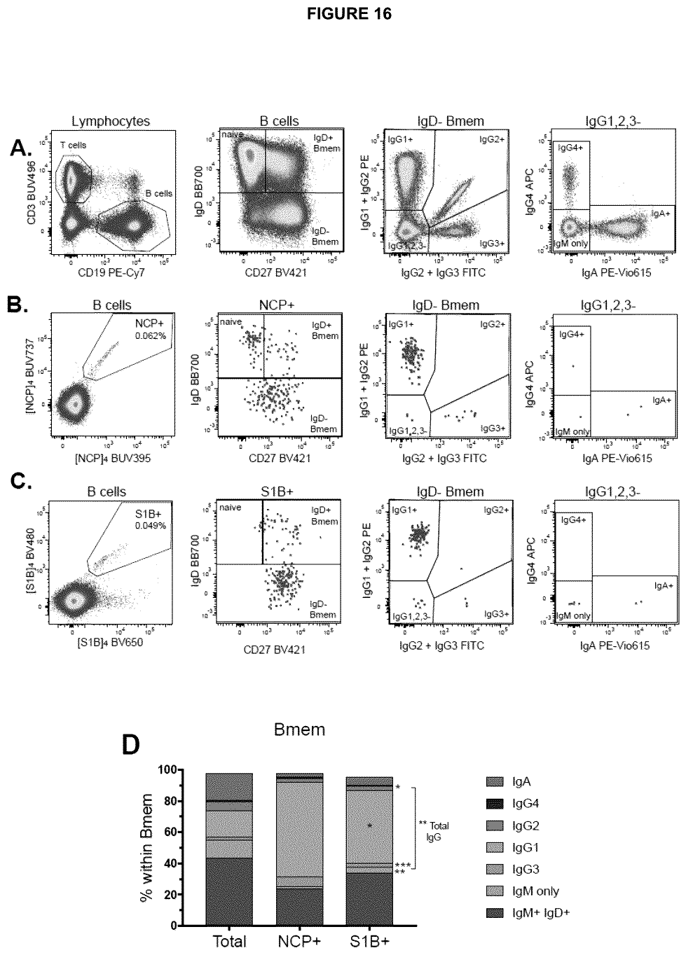

FIG. 16 shows the immunophenotyping of COVID19 specific B cells. Stepwise gating of CD3+ T cells and CD19+ B cells (left panel), followed by double discrimination of COVID-19 nucleocapsid (NCP) specific B cells, and of COVID-19 S1 B specific B cells within the same flow cytometry staining is shown in FIGS. 16B and 16C. Subsetting of total B cells in the same staining to discriminate unswitched B cells (IgD+) from Ig-class switched memory B(mem) cells (IgD-), within switched Bmem, IgG1, IgG2, IgG3, IgG4 and IgA expression B cells can be distinguished (FIG. 16A). Subsetting of S1B-specific B cells in the same flow cytometry stain with a similar approach as in A is shown in FIG. 16B,C.

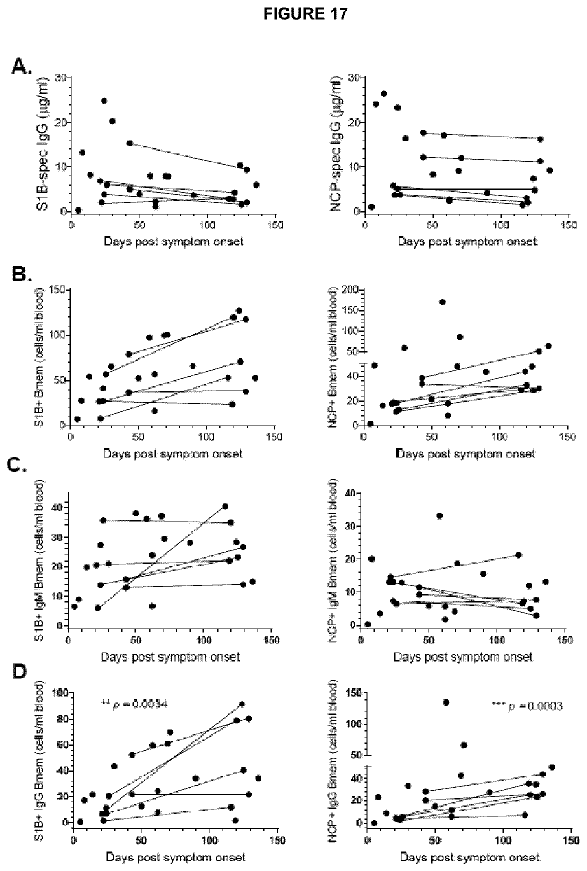

The antibody response to SARS-CoV2 infection can be measured after 4-7 days post-symptom onset and peaks around day 20. This is observed on our patient cohort using both the S1B and the NCP proteins as target (FIG. 17A). The subsequent decline in levels make can make it more difficult for sensitive detection of previous infection. In contrast, there is no decline after 20 days post-infection in the antigen-specific memory B cells in blood (FIG. 17B). In fact, the total S1 B-specific and the total NCP-specific Bmem trend to increase over time, at least until ˜140 days. This increase was more prominent in the IgG+ Bmem for both S1B-specific and NCP-specific Bmem, whereas IgM+ Bmem remained at similar levels (FIG. 17C, D).

### Example 6: Multiplex Allergen Stain to Detect Allergen Sensitization

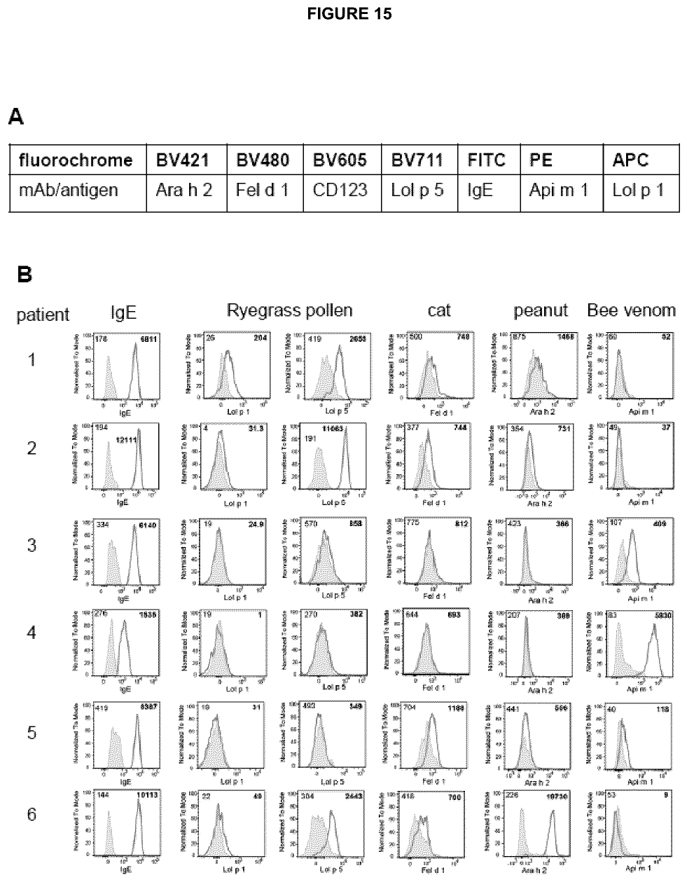

Multiplex allergen stain was performed to detect allergen sensitization on blood basophils using recombinant allergen tetramers (“Cytobas”). Using a 7-color flow panel, monoclonal antibodies CD123 and anti-IgE were used to identify blood basophils (CD123+ IgE+) and plasmacytoid dendritic cells (pDC; CD123+ IgE-), and on these cells, expression levels of IgE specific to Ara h 2, Fel d 1, Lol p 1, Lol p 5 and Api m 1 were determined (FIG. 15A).

Expression levels of IgE and specific IgE to 5 allergens on basophils and pDC of 6 individuals previously diagnosed with a form of allergy were determined (FIG. 15B). Patients 1 and 2 were diagnosed with ryegrass pollen allergy, patients 3 and 4 with bee venom allergy, patient 5 with cat dander allergy and patient 6 with peanut allergy. The ratio in median fluorescence intensity on (MFI) basophils (bold font) over MFI on pDC (regular font) can be used to quantify sensitization. This would omit the need for running a second staining with streptavidins to use as negative control.

The multiparameter basophil stain (CytoBas) shows the potential for differential diagnosis of allergen sensitization using molecular components (component resolved diagnostics; CRD) with the use of flow cytometry. This approach has advantages over the current basophil activation tests (BAT), as it does not require in vitro stimulation. Moreover, washed cells from fresh whole blood or fresh or frozen PBMC can be used. Finally, CytoBas can multiplex allergen components in a single flow cytometry tube without the need for serial dilutions.

CRD is currently implemented using microchip technology, which has proven difficult to implement widely in routine diagnostics. Flow cytometry is a standard test in many pathology laboratories, facilitating straightforward implementation of CytoBas.

1. A method of determining allergic reactivity in a subject, the method comprising:
providing a sample from a subject,
contacting the sample with an allergen linked to a detectable label in conditions for permitting the binding of the allergen to an IgE molecule present in the sample,
determining the binding of the allergen to an IgE molecule in the sample by detecting the label,
wherein the detection of the label indicates the subject has allergic reactivity.

2. A method according to claim 1, wherein the allergen is a recombinant allergen.

3. A method according to claim 1, wherein the allergen is a synthetic allergen.

4. A method according to claim 1, wherein the allergen is a natural allergen.

5. A method according to any one of claims 1 to 4, wherein the sample is a whole blood sample.

6. A method according to claims 1 to 5, wherein the IgE molecule is present on the surface of a cell.

7. A method according to claim 6, wherein the cell is an immune cell.

8. A method according to claim 7, wherein the immune cell is a basophil, eosinophil, mast cell or B cell.

9. A method according to any one of claims 6 to 8, wherein the method further comprises removing cells present in the blood sample that do not have an IgE molecule on their surface bound to the allergen linked to a detectable label.

10. A method according to any one of claims 1 to 9, wherein allergen linked to a detectable label that is not bound to IgE is removed.

11. A method according to any one of claims 1 to 10, wherein the method further comprises contacting the sample with a molecule that allows two or more immune cell types to be identified.

12. A method according to any one of claims 1 to 11, wherein the method further comprises contacting the sample with a molecule that distinguishes basophils from other cells.

13. A method according to any one of claims 1 to 11, wherein detecting the label is performed by flow cytometry.

14. A method according to any one or claims 1 to 13, wherein the allergen is a food-based allergen.

15. A method according to any one or claims 1 to 13, wherein the allergen is an airborne allergen or an environmental allergen.

16. A method according to any one of claims 1 to 13, wherein the allergen is an animal allergen.

17. A method according to any one of claims 1 to 13, wherein the allergen is a plant allergen.

18. A method according to any one or claims 1 to 13, wherein the allergen is an arthropod allergen.

19. A method according to claim 18, wherein the allergen is an insect allergen.

20. A method according to claim 18, wherein the allergen is a myriapod allergen.

21. A method according to claim 18, wherein the allergen is an arachnid allergen.

22. A method according to claim 18, wherein the allergen is a crustacean allergen.

23. A method according to any one of claims 1 to 22, wherein the allergen is an enzyme.

24. A method according to claim 23, wherein the enzyme has been modified to reduce its activity.

25. A method according to claim 24, wherein the modification is a point mutation or truncation.

26. A method according to any one of claims 1 to 25, wherein the sample is contacted with 2 or more allergens each linked to a different detectable label.

27. A method according to any one of claims 1 to 26, wherein the allergen is linked to a tag that facilitates binding to the detectable label.

28. A method according to claim 27, wherein the tag is Bir-A.

29. A method according to claim 28, wherein the Bir-A is biotinylated.

30. A method according to any one of claims 1 to 29, wherein the detectable label is fluorescent.

31. A method according to any one of claims 1 to 30, wherein the detectable label is linked to streptavidin.

32. A method of determining the efficacy of an allergy immunotherapy in a subject, the method comprising:
providing a first sample obtained from a subject before receiving allergy immunotherapy;
providing a second sample obtained from the subject after receiving allergy immunotherapy;
contacting the first and second samples from the subject with a recombinant or synthetic allergen linked to a label in conditions for permitting the binding of the allergen to an Ig molecule on the surface of a B cell present in the samples; and
determining the binding of the allergen to an Ig molecule on the surface of a B cell present in the first and second samples by detecting the label;
wherein an increase in the total number of, or proportion of, IgG-expressing B cells in the second sample relative to the first sample indicates efficacy of the allergy immunotherapy in the subject;
wherein an increase in the ratio of IgG: IgE-expressing B cells in the second sample relative to the first sample indicates efficacy of the allergy immunotherapy in the subject; or
wherein an increase in the total number of, or proportion of, IgG2 and/or IgG4-expressing B cells in the second sample relative to the first sample indicates efficacy of the allergy immunotherapy in the subject.

33. A method according to claim 32, wherein the method further comprises contacting the first and second blood samples with molecules that allow identification of B cells expressing IgM, IgA, IgG, IgD and IgE.

34. A method according to claim 32 or 33, wherein the blood sample is a whole blood sample.

35. A method according to any one of claims 32 to 34, wherein allergen linked to a detectable label that is not bound to Ig molecule is removed.

36. A method according to any one of claims 32 to 35, wherein the method further comprises contacting the sample with a molecule that allows two or more immune cell types to be identified.

37. A method according to any one of claims 32 to 36, wherein the method further comprises contacting the sample with a molecule that distinguishes B cells from other cells.

38. A method according to any one of claims 32 to 37, wherein detecting the label is performed by flow cytometry or CyToF.

39. A method according to any one or claims 32 to 38, wherein the allergen is a food-based allergen.

40. A method according to any one or claims 32 to 38, wherein the allergen is an airborne allergen or an environmental allergen.

41. A method according to any one of claims 32 to 38, wherein the allergen is an animal allergen.

42. A method according to any one of claims 32 to 38, wherein the allergen is a plant allergen.

43. A method according to any one or claims 32 to 38, wherein the allergen is an arthropod allergen.

44. A method according to claim 43, wherein the allergen is an insect allergen.

45. A method according to claim 43, wherein the allergen is a myriapod allergen.

46. A method according to claim 43, wherein the allergen is an arachnid allergen.

47. A method according to claim 43, wherein the allergen is a crustacean allergen.

48. A method according to any one of claims 32 to 47, wherein the allergen is an enzyme.

49. A method according to claim 48, wherein the enzyme has been modified to reduce its activity.

50. A method according to claim 49, wherein the modification is a point mutation or truncation.

51. A method according to any one of claims 32 to 50, wherein the sample is contacted with 2 or more allergens each linked to a different detectable label.

52. A method according to any one of claims 32 to 50, wherein the allergen is linked to a tag that facilitates binding to the detectable label.

53. A method according to claim 52, wherein the tag is Bir-A.

54. A method according to claim 53, wherein the Bir-A is biotinylated.

55. A method according to any one of claims 32 to 54, wherein the detectable label is fluorescent.

56. A method according to any one of claims 32 to 55, wherein the detectable label is linked to streptavidin.

57. A method according to any one of claims 1 to 56, wherein the samples are contacted with 2 or more allergens each linked to a different detectable label.

58. A recombinant polypeptide comprising an amino acid sequence encoding an allergen and a tag, wherein the tag facilitates linkage to a detectable label.

59. A recombinant polypeptide of claim 58, wherein the tag is a Bir-A tag.

60. A recombinant polypeptide of claim 59, wherein the Bir-A tag is biotinylated.

61. A recombinant polypeptide of any one of claims 58 to 60, wherein the detectable label is linked via any one of the group consisting of streptavidin and derivatives thereof, avidin and derivatives thereof, biotin, immunoglobulins, monoclonal antibodies, polyclonal antibodies, recombinant antibodies, antibody fragments and derivatives thereof, leucine zipper domain of AP-1, jun, fos, hexa-his, hexa-hat glutathione S-transferase, glutathione affinity, Calmodulin-binding peptide, Strep-tag, Cellulose Binding Domain, Maltose Binding Protein, S-Peptide-Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, preferably Con A or WGA and tetranectin or Protein A and Protein G.

62. A recombinant polypeptide of any one of claims 58 to 61, wherein the detectable label is a fluorescent tag.

63. A recombinant polypeptide of any one of claims 58 to 62, wherein the allergen is a food-based allergen.

64. A recombinant polypeptide of any one of claims 58 to 62, wherein the allergen is an airborne allergen or an environmental allergen.

65. A recombinant polypeptide of any one of claims 58 to 62, wherein the allergen is a plant allergen.

66. A recombinant polypeptide of any one of claims 58 to 62, wherein the allergen is an animal allergen.

67. A recombinant polypeptide of any one of claims 58 to 62, wherein the allergen is an arthropod allergen.

68. A method according to claim 67, wherein the allergen is an insect allergen.

69. A method according to claim 67, wherein the allergen is a myriapod allergen.

70. A method according to claim 67, wherein the allergen is an arachnid allergen.

71. A method according to claim 67, wherein the allergen is a crustacean allergen

72. A recombinant polypeptide of any one of claims 58 to 71, wherein the allergen is an enzyme.

73. A recombinant polypeptide of claim 72, wherein the enzyme has been modified to reduce its activity.

74. A recombinant polypeptide of claim 73, wherein the modification is a point mutation or truncation.

75. A nucleic acid comprising a nucleotide sequence encoding the polypeptide of any one of claims 58 to 74.

76. A vector comprising the nucleic acid of claim 75.

77. A host cell comprising the nucleic acid of claim 75 or the vector of claim 76.

78. A kit for use in any one of the methods of claims 1 to 57, the kit comprising one or more recombinant allergens, a detectable label, together with instructions for use, buffer, and/or control samples.

79. A method of detecting antigen-specific B cells in a subject, the method comprising:
providing a sample from a subject,
contacting the sample with an antigen linked to a detectable label in conditions for permitting the binding of the antigen to an Ig molecule on the surface of a B cell present in the sample, and
determining the binding of the antigen to an Ig molecule in the sample by detecting the label,
wherein the detection of the label indicates the subject has antigen-specific B cells.

80. A method of claim 79, wherein the antigen is from a vaccine.

81. A method of claim 79, wherein the antigen is from a pathogen or infectious agent.

82. A method of claim 81, wherein the antigen is from a virus, bacterium, fungi, protozoa or parasite.

83. A method of claim 82, wherein the antigen is from a virus.

84. A method of claim 83, wherein the virus is associated with, or cause, respiratory conditions or diseases.

85. A method of claim 83 or 84, wherein the antigen is a nucleocapsid protein or spike protein, or a domain within those proteins.

86. A method of claim 85, wherein the antigen is from SARS-CoV-2.

87. A method of claim 86, wherein the virus may be selected from: measles, polio, coronavirus, influenza, parainfluenza, respiratory syncytial virus (RSV), adenovirus, cytomegalovirus (CMV), Epstein-Barr virus (EBV), varicella zoster virus (VZV), dengue virus, rhinovirus, Herpes simplex virus and enteroviruses. More preferably, the virus is coronavirus or influenza. Even more preferably, the virus is severe acute respiratory syndrome coronavirus (SARS-CoV), Middle East respiratory syndrome coronavirus (MERS-CoV), or severe acute respiratory syndrome coronavirus 2 (SARS-CoV-2).

88. A method of claim 87, wherein the virus is SARS-CoV-2.

89. A method of claim 82, wherein the infectious agent is a bacterium, preferably, Clostridium tetani or Corynebacterium diphtheria.

90. A method of claim 79, wherein the agent is related to an auto-antigen.


SEQUENCE LISTING (90 sequences)

SEQ ID NO: 1 - protein, 19 aa, Artificial Sequence
  Bacteria ssFlg leader
MIKFLSALILLLVTTAAQA

SEQ ID NO: 2 - protein, 33 aa, Artificial Sequence
  Insect Api m 1 leader
MQVVLGSLFLLLLSTSHGWQIRDRIGDNELEER

SEQ ID NO: 3 - protein, 17 aa, Artificial Sequence
  Mammal Ig leader
MVLSLLYLLTALPGILS

SEQ ID NO: 4 - protein, 6 aa, Artificial Sequence
  6His Tag
HHHHHH

SEQ ID NO: 5 - protein, 15 aa, Artificial Sequence
  BirA Tag 1
LHHILDAQKMVWNHR

SEQ ID NO: 6 - protein, 15 aa, Artificial Sequence
  BirA Tag 2
GLNDIFEAQKIEWHE

SEQ ID NO: 7 - protein, 451 aa, Artificial Sequence
  TTC
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYDTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTDEGWTND

SEQ ID NO: 8 - protein, 536 aa, Artificial Sequence
  CRM197
GADDVVDSSKSFVMENFSSYHGTKPGYVDSIQKGIQKPKSGTQGNYDDDWKGEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 9 - protein, 225 aa, Artificial Sequence
  HBsAg
ENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 10 - protein, 550 aa, Artificial Sequence
  HA_A-Michigan-2015
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGSGYIPEAPRDGQAYVRKDGEWVLLSTFLGS

SEQ ID NO: 11 - protein, 566 aa, Artificial Sequence
  HA_B-Phuket-2013
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTGSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITGSGYIPEAPRDGQAYVRKDGEWVLLSTFLGS

SEQ ID NO: 12 - protein, 180 aa, Artificial Sequence
  Api m 1
APPKAVLKLEPPWINVLREDSVTLTCGGAHSPDSDSTQWFHNGNLIPTHTQPSYRFKANNNDSGEYRCQTGRTSLSDPIHLTVLSEWLALQTPHLEFREGETIMLRCHSWKDKPLIKVTFFQNGISKKFSHQMDPNFSIPQANHSHSGDYHCTGNIGYTPYSSKPVTITVQVPSVGSSSP

SEQ ID NO: 13 - protein, 175 aa, Artificial Sequence
  Api m 10
ERDQMMAATFDFPSLSFEDSDEGSNWNWNTLLRPNFLDGWYQTLQTHMKKVREQMAGILSRIPEQGVVNWNKIPEGANTTSTTKIIDGHVVTINETTYTDGSDDYSTLIRVRVIDVRPQNETILTTVSSEADSDVTTLPTLIGKNETSTQSSRSVESVEDFDNEIPKNQGDVLTA

SEQ ID NO: 14 - protein, 302 aa, Artificial Sequence
  Ves v 1
GPKCPFNSDTVSIIIETRENRNRDLYTLQTLQNHPEFKKKTITRPVVFITHGFTSSASETNFINLAKALVDKDNYMVISIDWQTAACTNEAAGLKYLYYPTAARNTRLVGQYIATITQKLVKHYKISMANIRLIGHSGLGAHASGFAGKKVQELKLGKYSEIIGLDAPARPSFDSNHCSERLCETDAEYVQIIHTSNYLGTEKTLGTVDFYMNNGKNQPGCGRFFSEVCSHSRAVIYMAECIKHECCLIGIPKSKSSQPISSCTKQECVCVGLNAKKYPSRGSFYVPVESTAPFCNNKGKII

SEQ ID NO: 15 - protein, 204 aa, Artificial Sequence
  Ves v 5
NNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFMNEELYQTK

SEQ ID NO: 16 - protein, 241 aa, Artificial Sequence
  Lol p 1
IAKVPPGPNITAEYGDKWLDAKSTWYGKPTGAGPKDNGGACGYKNVDKAPFNGMTGCGNTPIFKDGRGCGSCFEIKCTKPESCSGEAVTVTITDDNEEPIAPYHVFDLSGHAFGSMAKKGEEQNVRSAGELELQFRRVKCKYPDDTKPTFHVEKASNPNYLAILVKYVDGDGDVVAVDIKEKGKDKWTELKESWGAVWRIDTPDKLTGPFTVRYTTEGGTKSEFEDVIPEGWKADTSYSAK

SEQ ID NO: 17 - protein, 282 aa, Artificial Sequence
  Lol p 5
ADAGYTPAAAATPATPAATPAAAGGKATTDEQKLLEDVNAGFKAAVAAAANAPPADKFKIFEAAFSESSKGLLATSAAKAPGLIPKLDTAYDVAYKAAEATPEAKYDAFVTALTEALRVIAGALEVHAVKPATEEVLAAKIPTGELQIVDKIDAAFKIAATAANAAPTNDKFTVFESAFNKALNECTGGAYETYKFIPSLEAAVKQAYAATVAAAPEVKYAVFEAALTKAITAMTQAQKAGKPAAAAATAAATVATAAATAAAVLPPPLLVVQSLISLLIYY

SEQ ID NO: 18 - protein, 241 aa, Artificial Sequence
  Phl p 1
IPKVPPGPNITATYGDKWLDAKSTWYGKPTAAGPKDNGGACGYKDVDKPPFSGMTGCGNTPIFKSGRGCGSCFEIKCTKPEACSGEPVVVHITDDNEEPIAAYHVFDLSGIAFGSMAKKGDEQKLRSAGEVEIQFRRVKCKYPEGTKVTFHVEKGSNPNYLALLVKFVAGDGDVVAVDIKEKGKDKWIALKESWGAIWRIDTPEVLKGPFTVRYTTEGGTKGEAKDVIPEGWKADTAYESK

SEQ ID NO: 19 - protein, 287 aa, Artificial Sequence
  Phl p 5
ADLGYGPATPAAPAAGYTPATPAAPAEAAPAGKATTEEQKLIEKINAGFKAALAAAAGVQPADKYRTFVATFGAASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 20 - protein, 160 aa, Artificial Sequence
  Bet v 1
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 21 - protein, 245 aa, Artificial Sequence
  Cyn d 1
AMGDKPGPNITATYGDKWLDAKATFYGSDPRGAAPDDHGGACGYKDVDKAPFDGMTGCGNEPIFKDGLACGSCYEIKCKEPAECSGEPVLIKITDKNYEHIAAYHVFDLSGKAFGAMAKKGEEDKLRKAGELMLQFRRVKCEYPSDTKIAFHVEKGSSPNYLALLVKYAAGDGNIVGVDIKPKGSDEFLPMKQSWGAIWRIDPPKPLKGPFTIRLTSESGGHVEQDDVIPEDWKPDTVYKSKIQF

SEQ ID NO: 22 - protein, 243 aa, Artificial Sequence
  Pas n 1
GPPKVPPGPNITTNYNGKWLPAKATWYGQPNGAGPDDNGGACGIKNVNLPPYNGFTACGNPPIFKDGKGCGSCYEIRCNKPECSGQPVTVFITDMNYEPIAPYHVFDLSGKAFGAMAKPGLNDKLRHYGIFDLEFRRVRCKYQGGQKIVFHVEKGSNPNYLAMLVKFVADDGDIVLMELKEKSSDWKPMKLSWGAIWRMDTPKALVPPFSIRLTSESGKKVIAQDVIPVNWKPDTVYNSNVQF

SEQ ID NO: 23 - protein, 371 aa, Artificial Sequence
  Amb n 1
AEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 24 - protein, 133 aa, Artificial Sequence
  Par j 1
MVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 25 - protein, 102 aa, Artificial Sequence
  Par j 2
EEACGKVVQDIMPCLHFVKGEEKEPSKECCSGTKKLSEEVKTTEQKREACKCIVRATKGISGIKNELVAEVPKKCDIKTTLPPITADFDCSKIQSTIFRGYY

SEQ ID NO: 26 - protein, 301 aa, Artificial Sequence
  Der p 1
SSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCSWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 27 - protein, 129 aa, Artificial Sequence
  Der p 2
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 28 - protein, 162 aa, Artificial Sequence
  Fel d 1
VKMAETCPIFYDVFFAVANGNELLLDLSLTKVNATEPERTAMKKIQDCYVENGLISRVLDGLVMTTISSSKDCMGEAVQNTVEDLKLNTLGREICPAVKRDVDLFLTGTPDEYVEQVAQYKALPVVLENARILKNCVDAKMTEEDKENALSVLDKIYTSPLC

SEQ ID NO: 29 - protein, 156 aa, Artificial Sequence
  Can f 1
QDTPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 30 - protein, 186 aa, Artificial Sequence
  Gal d 1
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 31 - protein, 385 aa, Artificial Sequence
  Gal d 2
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 32 - protein, 123 aa, Artificial Sequence
  Bos d 4
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 33 - protein, 162 aa, Artificial Sequence
  Bos d 5
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 34 - protein, 601 aa, Artificial Sequence
  Ara h 1
KSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 35 - protein, 151 aa, Artificial Sequence
  Ara h 2
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 36 - protein, 510 aa, Artificial Sequence
  Ara h 3
ISFRQQPEENACQFQRLNAQRPDNRIESEGGYIETWNPNNQEFECAGVALSRLVLRRNALRRPFYSNAPQEIFIQQGRGYFGLIFPGCPSTYEEPAQQGRRYQSQRPPRRLQEEDQSQQQQDSHQKVHRFNEGDLIAVPTGVAFWLYNDHDTDVVAVSLTDTNNNDNQLDQFPRRFNLAGNHEQEFLRYQQQSRQSRRRSLPYSPYSPHSRPRREEREFRPRGQHSRRERAGQEEEDEGGNIFSGFTPEFLEQAFQVDDRQIVQNLWGENESEEEGAIVTVRGGLRILSPDGTRGADEEEEYDEDQYEYHEQDGRRGRGSRGGGNGIEETICTACVKKNIGGNRSPHIYDPQRWFTQNCHDLNLLILRWLGLSAEYGNLYRNALFVPHYNTNAHSIIYALRGRAHVQVVDSNGNRVYDEELQEGHVLVVPQNFAVAGKSQSENFEYVAFKTDSRPSIANFAGENSFIDNLPEEVVANSYGLPREQARQLKNNNPFKFFVPPFQQSPRAVA

SEQ ID NO: 37 - protein, 124 aa, Artificial Sequence
  Ara h 6
MRRERGRQGDSSSCERQVDGVNLKPCEQHIMQRIMGEQEQYDSYNFGSTRSSDQQQRCCDELNEMENTQRCMCEALQQIMENQCDGLQDRQMVQHFKRELMNLPQQCNFGAPQRCDLDVSGGRC

SEQ ID NO: 38 - protein, 157 aa, Artificial Sequence
  Ara h 8
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

SEQ ID NO: 39 - protein, 493 aa, Artificial Sequence
  Cor a 9
INVGLRRQQQRYFGECNLDRLNALEPTNRIEAEACQIESWDHNDQQFQCAGVAVIRRTIEPNGLLLPQYSNAPELIYIERGRGITGVLFPGCPETFEDPQQQSQQGQRQGQGQSQRSEQDRHQKIRHFREGDIIALPAGVAHWCYNDGDSPVVTVSLLHTNNYANQLDENPRHFYLAGNPDDEHQRQGQQQFGQRRRQQQHSHGEQGEQEQQGEGNNVFSGFDAEFLADAFNVDVDTARRLQSNQDKRRNIVKVEGRLQVVRPERSRQEWERQERQERESEQERERQRRQGGRGRDVNGFEETICSLRLRENICTRSRADIYTEQVGRINTVNSNTLPVLRWLQLSAERGDLQREGLYVPHWNLNAHSVVYAIRGRARVQVVDDNGNTVFDDELRQGQVLTIPQNFAVAKRAESEGFEWVAFKTNDNAQISPLAGRTSAIRALPDDVLANAFQISREEARRLKYNRQETTLVRSSRSSSERKRRSESEGRAEA

SEQ ID NO: 40 - protein, 125 aa, Artificial Sequence
  Cor a 14
FRTTITTVDVDEDIVNQQGRRGESCREQAQRQQNLNQCQRYMRQQSQYGSYDGSNQQQQQELEQCCQQLRQMDERCRCEGLRQAVMQQQGEMRGEEMREVMETARDLPNQCRLSPQRCEIRSARF

SEQ ID NO: 41 - protein, 118 aa, Artificial Sequence
  Ana o 3
SIYRAIVEVEEDSGREQSCQRQFEEQQRFRNCQRYVKQEVQRGGRYNQRQESLRECCQELQEVDRRCRCQNLEQMVRQLQQQEQIKGEEVRELYETASELPRICSISPSQGCQFQSSY

SEQ ID NO: 42 - protein, 124 aa, Artificial Sequence
  Jug r 1
FRTTITTMEIDEDIDNPRRRGEGCREQIQRQQNLNHCQYYLRQQSRSGGYDEDNQRQHFRQCCQQLSQMDEQCQCEGLRQVVRRQQQQQGLRGEEMEEMVQSARDLPNECGISSQRCEIRRSWF

SEQ ID NO: 43 - protein, 124 aa, Artificial Sequence
  Ber e 1
FRATVTTTVVEEENQEECREQMQRQQMLSHCRMYMRQQMEESPYQTMPRRGMEPHMSECCEQLEGMDESCRCEGLRMMMMRMQQEEMQPRGEQMRRMMRLAENIPSRCNLSPMRCPMGGSIAGF

SEQ ID NO: 44 - protein, 132 aa, Artificial Sequence
  Ses i 1
TTYTTTVTTTAIDDEANQQSQQCRQQLQGRQFRSCQRYLSQGRSPYGGEEDEVLEMSTGNQQSEQSLRDCCQQLRNVDERCRCEAIRQAVRQQQQEGGYQEGQSQQVYQRARDLPRRCNMRPQQCQFRVIFV

SEQ ID NO: 45 - protein, 136 aa, Artificial Sequence
  Fag e 3
GDYPFEPRRQIEHCRQRCQDRSQGFRETRECLETCEKRAGFDDEPSKEVDSYTTDTRSRDPQQEFRQCRHRCQTQEEGGRQQRKCEQRCEKQLERQQGYDDEEFGQGRSNIGAPVRNYDDCTEMCGGSPLCALFCE

SEQ ID NO: 46 - protein, 531 aa, Artificial Sequence
  Pru du 6
ARQSQLSPQNQCQLNQLQAREPDNRIQAEAGQIETWNFNQEDFQCAGVAASRITIQRNGLHLPSYSNAPQLIYIVQGRGVLGAVFSGCPETFEESQQSSQQGRQQEQEQERQQQQQGEQGRQQGQQEQQQERQGRQQGRQQQEEGRQQEQQQGQQGRPQQQQQFRQFDRHQKTRRIREGDVVAIPAGVAYWSYNDGDQELVAVNLFHVSSDHNQLDQNPRKFYLAGNPENEFNQQGQSQPRQQGEQGRPGQHQQPFGRPRQQEQQGSGNNVFSGFNTQLLAQALNVNEETARNLQGQNDNRNQIIRVRGNLDFVQPPRGRQEREHEERQQEQLQQERQQQGGQLMANGLEETFCSLRLKENIGNPERADIFSPRAGRISTLNSHNLPILRFLRLSAERGFFYRNGIYSPHWNVNAHSVVYVIRGNARVQVVNENGDAILDQEVQQGQLFIVPQNHGVIQQAGNQGFEYFAFKTEENAFINTLAGRTSFLRALPDEVLANAYQISREQARQLKYNRQETIALSSSQQRRAVV

SEQ ID NO: 47 - protein, 150 aa, Artificial Sequence
  Hev b 5
ASVEVESAATALPKNETPEVTKAEETKTEEPAAPPASEQETADATPEKEEPTAAPAEPEAPAPETEKAEEVEKIEKTEEPAPEADQTTPEEKPAEPEPVAEEEPKHETKETETEAPAAPAEGEKPAEEEKPITEAAETATTEVPVEKTEE

SEQ ID NO: 48 - protein, 187 aa, Artificial Sequence
  Hev b 6
EQCGRQAGGKLCPNNLCCSQWGWCGSTDEYCSPDHNCQSNCKDSGEGVGGGSASNVLATYHLYNSQDHGWDLNAASAYCSTWDANKPYSWRSKYGWTAFCGPVGAHGQSSCGKCLSVTNTGTGAKTTVRIVDQCSNGGLDLDVNVFRQLDTDGKGYERGHITVNYQFVDCGDSFNPLFSVMKSSVIN

SEQ ID NO: 49 - protein, 139 aa, Artificial Sequence
  Alt a 1
APLESRQDTASCPVTTEGDYVWKISEFYGRKPEGTYYNSLGFNIKATNGGTLDFTCSAQADKLEDHKWYSCGENSFMDFSFDSDRSGLLLKQKVSDDITYVATATLPNYCRAGGNGPKDFVCQGVADAYITLVTLPKSS

SEQ ID NO: 50 - protein, 157 aa, Artificial Sequence
  Asp f 1
APSPLDARATWTCINQQLNPKTNKWEDKRLLYNQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSH

SEQ ID NO: 51 - protein, 269 aa, Artificial Sequence
  Cla h 8
MPGQQATKHESLLDQLSLKGKVVVVTGASGPKGMGIEAARGCAEMGAAVAITYASRAQGAEENVKELEKTYGIKAKAYKCQVDSYESCEKLVKDVVADFGQIDAFIANAGATADSGILDGSVEAWNHVVQVDLNGTFHCAKAVGHHFKERGTGSLVITASMSGHIANFPQEQTSYFNVAKAAGCIHMARSLANEWRDFARVNSISPGYIDTGLSDFVPKETQQLWHSMIPMGRDGLAKELKGAYVYFASDASTYTTGADLLIDGGYTTR

SEQ ID NO: 52 - DNA, 591 bp, Artificial Sequence
  Api m 1
atgcaagtcgttctcggatccttgttccttctcctcctctctacctctcacggatggcaaatcagggataggatcggggataacgagttggaggaacggataatatatccaggaacgttatggtgcgggcatggtaacaagtcgtccggcccgaacgagctaggtcggttcaagcacacggatgcatgctgtcgaacccaggacatgtgcccggacgtgatgtcagctggtgaatcgaagcacggcctgaccaacacggcctcccacaccaggttgtcgtgcgactgcgacgacaagttctatgattgtcttaaaaattcggcggacacgattagctcgtatttcgtagggaagatgtacttcaatctgatagacacgaagtgttacaaactggagcatcctgtcaccgggtgcggtgagagaaccgagggtcgttgtcttcactacaccgtggacaaaagcaaaccgaaagtgtaccaatggttcgatcttcgcaagtatggaggcggaggcagccaccaccatcaccatcatccaggaggaggcctgaacgacatcttcgaggctcagaagatcgagtggcacgagtga

SEQ ID NO: 53 - DNA, 909 bp, Artificial Sequence
  Lol p 1
atgcaggtcgtgctgggttccctgttcctgctgctgctgtctacctctcacggctggcaaatccgtgaccgtatcggcgacaacgagctggaagaacgtatcgctaaggtgccacctggtcctaacatcaccgctgagtacggcgacaagtggctggacgctaagtccacttggtacggcaagcctaccggtgctggtcccaaggataacggtggtgcttgcggttacaagaacgtggacaaggctcccttcaacggcatgaccggttgcggcaacacccctatcttcaaggacggtcgtggttgcggctcctgcttcgagatcaagtgcaccaagcctgagtcctgctctggcgaggctgtgaccgtgactatcaccgacgacaacgaagaaccgatcgctccctacgtgttcgacctgtccggtcacgctttcggttccatggctaagaagggcgaagaacagaacgtgcgttccgctggcgaactcgagctgcaattccgtcgtgtgaagtgcaagtaccccgacgacaccaagccaaccttccacgtcgagaaggcttctaaccccaactacctggctatcctggtcaaatacgtggacggcgacggcgacgtggtggctgtggacatcaaagagaagggcaaagacaagtggaccgagctgaaagaatcctggggtgctgtgtggcgtatcgacacccctgacaagctgaccggtcctttcaccgtgcgttacaccaccgaaggtggtactaagtccgagttcgaggacgtgatccccgaaggctggaaggctgacacctcctactccgctaaaggtggtggtggttcccaccatcaccatcatcaccctggtggtggcctgaacgacatcttcgaggctcaaaagatcgagtggcacgaataa

SEQ ID NO: 54 - DNA, 1716 bp, Artificial Sequence
  HA_A-Michigan-2015
atgaaggccatcctggtggtgctgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggattctgggcaaccctgagtgtgaaagcctgagcacagccagcagctggtcctacatcgtggaaaccagcaacagcgacaacggcacatgcttccccggcgacttcatcaactacgaggaactgcgggaacagctgagcagcgtgtccagcttcgagagattcgagatcttccccaagacctccagctggcccaaccacgattctaacaagggcgtgacagccgcctgtcctcatgccggcgctaagagcttctacaagaacctgatctggctggtcaagaagggcaacagctaccccaagctgaaccagagctacatcaacgacaagggcaaagaggtgctggtcctctggggcatccaccatcctagcacaacagccgatcagcagagcctgtaccagaacgccgatgcctatgtgttcgtgggcaccagccggtacagcaagaagttcaagcccgagatcgccaccagacctaaagtgcgggacagagaaggccggatgaactactactggaccctggtggaacccggcgacaagatcacatttgaggccaccggcaacctggtggtccccagatacgccttcaccatggaaagaaatgccggcagcggcatcatcatcagcgacacccctgtgcacgactgcaacaccacctgtcagacacctgagggcgccatcaataccagcctgcctttccagaacattcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgagctgctggtgctcctggaaaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaaactgaaccgcgagaagatcgacggctccggatatatccctgaggctcctagagatggccaggcctacgtcagaaaggatggcgagtgggtcctgctgagcacctttctcggatccggcctgaacgacatcttcgaggcccagaaaatcgagtggcacgagggccaccaccatcatcaccattga

SEQ ID NO: 55 - DNA, 1764 bp, Artificial Sequence
  HA_B-Phuket-2013
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagctacttcgccaacctgaagggcaccagaaccagaggcaagctgtgccccgattgcctgaactgcaccgatctggatgtggccctgggcagacctatgtgcgtgggaacaacacctagcgccaaggccagcatcctgcatgaagtgcggcctgtgaccagcggctgcttccctattatgcacgaccggaccaagatcagacagctgcccaatctgctgcggggctacgagaagatcaggctgagcacccagaacgtgatcgacgccgaaaaagctcctggcggcccttacagactgggcggatctggctcttgccccaacgctacaagcaagatcggcttcttcgccaccatggcctgggccgtgcctaaggacaactacaagaacgccaccaatcctctgaccgtggaagtgccctacatctgtaccgaaggcgaggaccagatcaccgtgtggggcttccacagcgacaacaagacccagatgaagtccctgtacggcgacagcaaccctcagaagtttaccagcagcgccaacggcgtgaccacacactatgtgtcccagatcggcgacttccccgaccagacagaagatggcggactgcctcagagcggcagaatcgtggtggactacatgatgcagaagcccggcaagaccggcaccatcgtgtatcagagaggcgtcctgctgccacagaaagtttggtgcgccagcggccggtccaaagtgatcaaaggatctctgcctctgatcggcgaggccgactgtctgcacgaagaatatggcggcctgaacaagagcaagccctactacacaggcaagcacgccaaagccatcggcaactgccctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggacccagcgccgtggatatcggcaatggctgcttcgagacaaagcacaagtgcaaccagacctgcctggacagaattgccgccggaacctttaacgccggcgagtttagcctgcctaccttcgacagcctgaacatcacaggctccggatatatccctgaggctcctagagatggccaggcctacgtcagaaaggatggcgagtgggtcctgctgagcacctttctcggatccggcctgaacgacatcttcgaggcccagaaaatcgagtggcacgagggccaccaccatcatcaccattga

SEQ ID NO: 56 - protein, 550 aa, Artificial Sequence
  HA_A_Brisbane-2018
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYFPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGSGYIPEAPRDGQAYVRKDGEWVLLSTFLGS

SEQ ID NO: 57 - protein, 425 aa, Artificial Sequence
  N_SARS-Cov2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKGGPRQGRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 58 - protein, 422 aa, Artificial Sequence
  N_SARS-Cov
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 59 - protein, 1219 aa, Artificial Sequence
  S_SARS-Cov2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 60 - protein, 681 aa, Artificial Sequence
  S1_SARS-Cov2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 61 - protein, 214 aa, Artificial Sequence
  S1B_SARS-Cov2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 62 - DNA, 1032 bp, Artificial Sequence
  Lol p 5
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcgctgacgctggttacactccagctgctgcagctacccctgctactcctgccgctacaccagctgccgctggcggaaaagctactaccgacgagcaaaagctgttggaggacgtgaacgctggtttcaaggctgctgttgctgctgccgctaacgctcctcctgctgacaagttcaagatcttcgaagctgctttctccgagtcctccaagggcctgctggctacttctgctgctaaggctcctggactgatccccaagctggacaccgcttacgacgtggcctacaaggctgccgaggctactccagaggctaagtacgacgctttcgtgaccgctctgaccgaggctttgcgtgtgatcgctggtgctctggaagtgcacgctgtgaagcctgctaccgaggaagtgctggctgctaagatccctactggcgagctgcagatcgtggacaagatcgacgctgccttcaagatcgctgctaccgctgcaaacgctgctcccaccaacgataagttcaccgtgttcgagtccgctttcaacaaggctctgaacgagtgcaccggtggtgcttacgagacttacaagttcatcccatccttggaggctgccgtgaagcaggcttacgctgctactgtagctgctgctcccgaagtgaagtacgctgtgttcgaggctgccctgaccaaggctatcaccgctatgactcaggctcagaaggctggcaagcctgctgcggctgctgctacagccgctgcaactgttgctacagcagcggccactgcagctgccgttttgcctcctcctctgctggtggtgcagtccctgatctccctgctgatctactactaa

SEQ ID NO: 63 - DNA, 906 bp, Artificial Sequence
  Phl p 1
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcatccctaaggttccacctggtcctaacatcaccgctacctacggcgacaagtggctggacgctaagtccacttggtacggcaagcctaccggtgctggtcccaaggataacggtggtgcttgcggttacaaggacgtggacaagcctcctttctccggcatgaccggttgcggcaacacccctatcttcaagtccggtcgtggttgcggttcctgcttcgagatcaagtgcaccaagcctgaggcctgctctggcgaacctgtggtggtgcacatcaccgacgacaacgaggaacctatcgctccctacgtgttcgacctgtccggtcacgctttcggtgctatggctaagaagggcgacgagcaaaagctgcgttccgctggcgaattggagctgcagttccgtcgtgtgaagtgcaagtaccccgagggcaccaaagtgaccttccacgtcgagaagggttctaaccccaactacctggctctgctggtcaaatacgtgaacggcgacggcgacgtggtggctgtggacatcaaagagaagggcaaagacaagtggatcgagctgaaagagtcctggggcgctatctggcgtatcgacactcccgacaagctgaccggtcctttcaccgtgcgttacaccaccgaaggtggcaccaagaccgaagctgaggacgtgatccctgaaggctggaaggctgacacctcctacgagtccaaataa

SEQ ID NO: 64 - DNA, 852 bp, Artificial Sequence
  Der p 1
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcactaacgcttgctccatcaacggcaacgctcccgctgaaatcgacctgcgtcagatgcgtaccgtgactcccatccgtatgcaaggcggttgcggttcctcttgggctttctccggtgtcgctgctaccgagtctgcttacctggcttaccgcaatcagtccctggacctggctgagcaagaactggtggactgcgcttctcagcacggttgtcacggcgacactatccctcgtggtatcgagtacatccagcacaacggtgtcgtgcaagagtcctactaccgttacgtggcccgcgagcagtcttgcagacgtcctaacgctcagcgtttcggtatctccaactactgccagatctaccctcctaacgtgaacaagatccgcgaggctctggctcagacccactctgctatcgctgtgatcatcggtatcaaggacctggacgctttccgtcactacgacggtcgtaccatcatccagcgtgacaacggttaccagcctaactaccacgctgtcaacatcgtgggctactccaacgctcagggtgtcgactactggatcgtgcgcaactcctgggacaccaactggggagacaacggctacggttacttcgctgctaacatcgacctgatgatgatcgaggaatacccctacgtggtcatcctctaa

SEQ ID NO: 65 - DNA, 573 bp, Artificial Sequence
  Der p 2
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcgaccaagtggatgtgaaggactgcgctaaccacgagatcaagaaggtgctggtccccggttgccacggttctgagccttgcatcatccaccgtggaaagcccttccagttggaggctgtgttcgaggctaaccagaacaccaagaccgctaagatcgaaatcaaggcttccatcgacggcctggaagtggacgtgcccggtatcgatcctaacgcttgccactacatgaagtgccctctggtcaagggccagcagtacgacatcaagtacacctggaacgtgcccaagatcgctcccaagtccgagaacgtggtggtcaccgtgaaagtgatgggcgacgacggtgtcctggcttgcgctattgctacccacgccaagatccgtgactaa

SEQ ID NO: 66 - DNA, 612 bp, Artificial Sequence
  Fel d 1
atgagaggcgctctgctggtgctggctctgcttgttacacaggccctaggagtgaagatggccgagacatgccccatcttctacgacgtgttcttcgccgtggccaacggcaatgagctgctgctggatctgagcctgaccaaagtgaacgccaccgagcctgagagaaccgccatgaagaagatccaggactgctacgtggaaaacggcctgatcagcagagtgctggacggcctggtcatgacaaccatcagcagctccaaggactgcatgggcgaagccgtgcagaacaccgtggaagatctgaagctgaacaccctgggccgcgagatttgtcccgccgtgaagagagatgtggacctgttcctgaccggcacacccgacgagtatgtggaacaggtggcccagtataaggccctgcctgtggtgctggaaaacgccagaatcctgaagaactgcgtggacgccaagatgaccgaagaggacaaagagaatgccctgagcctgctggacaagatctacacaagccctctgtgcggatccggcctgaacgacatcttcgaggcccagaaaatcgagtggcacgagggccaccaccatcatcaccattga

SEQ ID NO: 67 - DNA, 639 bp, Artificial Sequence
  Ara h 2
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcagacagcaatgggaattgcaaggcgaccgtcgttgccagtctcagctcgaacgtgctaacctgaggccttgcgagcagcacctgatgcagaagatccaacgcgacgaggactcctacggtcgtgacccttacagcccttctcaggatccatactcaccctctcaagaccctgaccgtcgcgacccctattctccctctccttacgatcgtcgcggtgctggttcctctcagcaccaagaaaggtgctgcaacgaactgaacgagttcgagaacaaccagcgttgcatgtgcgaggctctgcagcagatcatggaaaaccagtccgaccgtctgcagggtcgtcagcaagagcagcagttcaagcgcgagctgcgtaacctgcctcagcaatgcggactgcgtgctcctcaacgttgcgacctggaagtggaatccggtggacgtgaccgttactaa

SEQ ID NO: 68 - DNA, 1035 bp, Artificial Sequence
  Pen m 1
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagcgacgctatcaagaaaaagatgcaggccatgaagctggaaaaggacaacgctatggaccgtgctgacaccctggaacagcagaacaaagaggctaacaaccgcgctgagaagtccgaggaagaggtgcacaacctgcaaaagcgtatgcagcagttggagaacgacctggaccaggtgcaagagtccctgctgaaggctaacatccagctggtcgagaaggacaaggctctgtccaacgctgagggcgaagtggctgctctgaaccgtcgtatccagctgctggaagaggacttggagcgttccgaggaacgtctgaacaccgctaccaccaagctggctgaggcttctcaggctgctgacgagtctgagcgtatgcgcaaggtgctggaaaaccgttctctgtccgacgaggaacgcatggacgctttggagaaccagctgaaagaggctcgtttcctggctgaagaggctgaccgtaaatacgacgaggtggcccgcaagctcgctatggtggaagctgacttggaacgcgctgaggaaagggctgagactggcgagtccaagatcgtggaactcgaagaggaactgcgtgtcgtgggcaacaacctgaagtccctggaagtgtccgaagagaaggccaaccagcgcgaagaggcttacaaagagcagatcaagaccctgaccaacaagctgaaggccgctgaggctcgtgctgagttcgctgaacgttccgtgcaaaagctgcagaaagaggtggaccgtttggaggacgagctggtcaacgagaaagagaagtacaagtccatcaccgacgagttggaccagaccttctccgagctgtccggttactaa

SEQ ID NO: 69 - DNA, 1380 bp, Artificial Sequence
  Nucleocapsid
atggtcctgagcctgctgtacctgcttacagccctgcctggcatcttaagcagcgacaacggccctcagaaccagcggaatgcccctagaatcacctttggcggccctagcgatagcaccggcagcaatcagaatggcgagagaagcggcgccagaagcaagcagagaaggcctcaaggcctgcctaacaacaccgccagctggttcacagccctgacacagcacggcaaagaggacctgaagttccctagaggacagggcgtgcccatcaacaccaacagcagccccgatgaccagatcggctactacagacgggccaccagaagaatcagaggcggcgacggcaagatgaaggatctgagccccagatggtacttctactacctcggcacaggacccgaagccggacttccttatggcgccaacaaggacggcatcatctgggttgcaacagaaggcgccctgaacacccctaaggaccacatcggcaccagaaatcccgccaacaatgccgccattgtgctgcagttgcctcagggcacaacactgcccaagggcttttacgccgagggaagcagaggcggatctcaggcctctagcagaagcagcagccggtccagaaacagctcccggaatagcacacctggcagcagcagaggcacaagccctgctagaatggctggcaacggcggagatgctgctctggcacttctgctgctcgaccggctgaatcagctggaaagcaagatgagcggcaagggccagcagcaacagggccagaccgtgacaaaaaagtctgccgccgaggccagcaagaagcccagacagaaaagaaccgccaccaaggcctacaacgtgacccaggcctttggcagaagaggccctgagcagacccagggcaatttcggcgatcaagagctgatcagacagggcaccgactacaagcactggcctcagatcgcccagtttgccccttctgccagcgccttctttggcatgagccggatcggcatggaagtgaccccatctggcacctggctgacatacacaggcgccatcaagctggacgacaaggaccccaacttcaaggaccaagtgatcctgctgaacaagcacatcgacgcctacaagacattccctccaaccgagcctaagaaggacaagaagaagaaggccgacgagacacaggccctgcctcagcgccagaaaaagcagcagactgtgacactgctgcccgctgccgacctggacgatttttctaagcagctgcagcagagcatgagcagcgccgattctacacaggctggatccggactgaacgacatcttcgaagcccagaaaatcgagtggcacgagggccaccaccatcatcaccattga

SEQ ID NO: 70 - DNA, 2124 bp, Artificial Sequence
  S1
atggtcctgagcctgctgtacctgcttacagccctgcctggcatcttaagcgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctatcagacccagacgaattctcccggatccggcctgaacgacatcttcgaggcccagaaaatcgagtggcacgagggccaccaccatcatcaccattga

SEQ ID NO: 71 - DNA, 768 bp, Artificial Sequence
  S1B
atgagaggcgctctgctggtgctggctctgcttgttacacaagctttaggacggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcggatccggcctgaacgacatcttcgaagctcagaaaatcgagtggcacgagggccaccaccatcatcaccattga

SEQ ID NO: 72 - protein, 15 aa, Artificial Sequence
  BirA Tag 2/AviTag
GLNDIFEAQKIEWHE

SEQ ID NO: 73 - protein, 17 aa, Artificial Sequence
  mammal Fel d 1 leader
MRGALLVLALLVTQALG

SEQ ID NO: 74 - protein, 18 aa, Artificial Sequence
  Insect Api m 1 short leader
MQVVLGSLFLLLLSTSHG

SEQ ID NO: 75 - protein, 17 aa, Artificial Sequence
  Mammal Fel d 1 leader
MRGALLVLALLVTQALG

SEQ ID NO: 76 - protein, 551 aa, Artificial Sequence
  HA_A-Brisbane-2018
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYFFPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGSGYIPEAPRDGQAYVRKDGEWVLLSTFLGS

SEQ ID NO: 77 - protein, 373 aa, Artificial Sequence
  Api m 3
ELKQINVIFRHGDRIPDEKNEMYPKDPYLYYDFYPLERGELTNSGKMREYQLGQFLRERYGDFLGDIYTEESVSALSSFYDRTKMSLQLVLAALYPPNKLQQWNEDLNWQPIATKYLRRYEDNIFLPEDCLLFTIELDRVLESPRGKYEFSKYDKLKKKLEEWTGKNITTPWDYYYIYHTLVAEQSYGLTLPSWTNNIFPRGELFDATVFTYNITNSTPLLKKLYGGPLLRIFTKHMLDVVSGTQKKKRKIYLFSGHESNIASVLHALQLYYPHVPEYSSSIIMELHNIEGTHYVKIVYYLGIPSEARELQLPGCEVLCPLYKYLQLIENVIPSNEELICDKRFVDESANNLSIEELDFVKLNLIRIAGTENK

SEQ ID NO: 78 - protein, 301 aa, Artificial Sequence
  Pol a 1
MSPDCTFNEKDIVFYVYSRDKRDGIILKKETLTNYDLFTKSTISKQVVFLIHGFLSTGNNENFVAMSKALIEKDDFLVISVDWKKGACNAFASTKDALGYSKAVGNTRHVGKFVADFTKLLVEKYKVLISNIRLIGHSLGAHTSGFAGKEVQKLKLGKYKEIIGLDPAGPYFHRSDCPDRLCVTDAEYVQVIHTSIILGVYYNVGSVDFYVNYGKNQPGCNEPSCSHTKAVKYLTECIKHECCLIGTPWKKYFSTPKPISQCRGDTCVCVGLNAKSYPARGAFYAPVEANAPYCHNEGIKL

SEQ ID NO: 79 - protein, 316 aa, Artificial Sequence
  Pol d 1
ADDLTTLRNGTLDRGITPDCTFNEKDIELHVYSRDKRNGIILKKEILKNYDLFQKSQISHQIAILIHGFLSTGNNENFDAMAKALIEIDNFLVISVDWKKGACNAFASTNDVLGYSQAVGNTRHVGKYVADFTKLLVEQYKVPMSNIRLIGHSLGAHTSGFAGKEVQRLKLGKYKEIIGLDPAGPSFLTNKCPNRLCETDAEYVQAIHTSAILGVYYNVGSVDFYVNYGKSQPGCSEPSCSHTKAVKYLTECIKRECCLIGTPWKSYFSTPKPISQCKRDTCVCVGLNAQSYPAKGSFYVPVDKDAPYCHNEGIKL

SEQ ID NO: 80 - protein, 206 aa, Artificial Sequence
  Pol d 5
NDYCKIKCSSGVHTVCQYGESTKPSKNCAGKLIKSVGPTEEEKKLIVEEHNRFRQKVAKGLETRGNPGPQPAASNMNNLVWNDELAKIAQVWASQCQILVHDKCRNTEKYQVGQNIAYAGSSNHFPSVTKLIQLWENEVKDFNYNTGITNKNFGKVGHYTQMVWGNTKEVGCGSLKYVEKNMQIHYLICNYGPAGNYLGQPIYTKK

SEQ ID NO: 81 - protein, 320 aa, Artificial Sequence
  Sol I 1
EPDPGVVEYLKQSCVYGNSSYINVYLYNSRFQGKNLGNQQSCQDINASLPVVFITHGFTSSAQVSTFKDLANAFVQKGHTAFIVDWSEAACTDGLPGVQFAEYNAAASNTYDIGQLMAKYTVDLMNKCKIPLNNIQYVGHSLGSHVCGFAAKHVKKLINKTMPYILALDPADPSFGSNKCGERICKSDAKRIVVFKTSILGIGENIIGHLLIVFDGGKSQPACSWYDVPCSHSESIVYATGMVSGRCQHLAVPWTAQQRINPIQWKFWRVFTSNIPAYPTSDTTNCVVLNTNVFKNDNTFEGEYHAFPDCARNLFKCRQQ

SEQ ID NO: 82 - protein, 86 aa, Artificial Sequence
  Myr p 1
KDLADPESEAVGFADAFGEADAVGEADPNAGLGSVFGRLARILGRVIPKVAKKLGPKVAKVLPKVMKEAIPMAVEMAKSQEEQQPQ

SEQ ID NO: 83 - protein, 48 aa, Artificial Sequence
  Myr p 2
KALADPESDAVGFADAVGEADPIDWKKVDWKKVSKKTCKVMLKACKFL

SEQ ID NO: 84 - protein, 63 aa, Artificial Sequence
  Myr p 3
PNVKAKALADPESDAVGFADAVGEADPFDITKLNIKKLTKATCKVISKGASMCKVLFDKKKQE

SEQ ID NO: 85 - protein, 92 aa, Artificial Sequence
  Ara h 9
ISCGQVNSALAPCIPFLTKGGAPPPACCSGVRGLLGALRTTADRQAACNCLKAAAGSLRGLNQGNAAALPGRCGVSIPYKISTSTNCATIKF

SEQ ID NO: 86 - protein, 420 aa, Artificial Sequence
  Tri a 19
SRLLSPRGKELHTPQEQFPQQQQFPQPQQFPQQQIPQQHQIPQQPQQFPQQQQFLQQQQIPQQQIPQQHQIPQQPQQFPQQQQFPQQHQSPQQQFPQQQFPQQKLPQQEFPQQQISQQPQQLPQQQQIPQQPQQFLQQQQFPQQQPPQQHQFPQQQLPQQQQIPQQQQIPQQPQQIPQQQQIPQQPQQFPQQQFPQQQFPQQQFPQQEFPQQQQFPQQQIARQPQQLPQQQQIPQQPQQFPQQQQFPQQQSPQQQQFPQQQFPQQQQLPQKQFPQPQQIPQQQQIPQQPQQFPQQQFPQQQQFPQQQEFPQQQFPQQQFHQQQLPQQQFPQQQFPQQQFPQQQQFPQQQQLTQQQFPRPQQSPEQQQFPQQQFPQQPPQQFPQQQFPIPYPPQQSEEPSPYQQYPQQQPSGSDVISISGL

SEQ ID NO: 87 - DNA, 804 bp, Artificial Sequence
  Der p 1
atgcaagtggtgctgggttccctgttcctgctgctgctgtctacctctcacggatcccaccaccaccatcaccacggttccggactgaacgacatcttcgaggctcaaaagatcgagtggcacgagggctcgagcactaacgcttgctccatcaacggcaacgctcccgctgaaatcgacctgcgtcagatgcgtaccgtgactcccatccgtatgcaaggcggttgcggttcctcttgggctttctccggtgtcgctgctaccgagtctgcttacctggcttaccgcaatcagtccctggacctggctgagcaagaactggtggactgcgcttctcagcacggttgtcacggcgacactatccctcgtggtatcgagtacatccagcacaacggtgtcgtgcaagagtcctactaccgttacgtggcccgcgagcagtcttgcagacgtcctaacgctcagcgtttcggtatctccaactactgccagatctaccctcctaacgtgaacaagatccgcgaggctctggctcagacccactctgctatcgctgtgatcatcggtatcaaggacctggacgctttccgtcactacgacggtcgtaccatcatccagcgtgacaacggttaccagcctaactaccacgctgtcaacatcgtgggctactccaacgctcagggtgtcgactactggatcgtgcgcaactcctgggacaccaactggggagacaacggctacggttacttcgctgctaacatcgacctgatgatgatcgaggaatacccctacgtggtcatcctctaa

SEQ ID NO: 88 - DNA, 1446 bp, Artificial Sequence
  Tri a 19
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagctcccgtttgctgtcccctcgtggaaaagagctgcacacccctcaagagcagttccctcagcagcagcaattcccacagcctcaacagttcccacaacagcaaatccctcaacagcatcagatcccgcagcagccccaacaatttccccagcaacagcagttcttgcagcaacaacagattccacagcagcagattcctcagcaacaccaaattccgcaacaaccccagcagtttccgcaacagcaacagtttccccaacaacatcaatctccccagcagcaatttccgcagcagcagttccctcaacagaagctgccccagcaagagttcccgcagcaacaaatcagccagcagcctcagcaattgcctcagcagcaacaaatccctcagcaaccacagcagttcctccaacaacagcaatttccacaacaacagccacctcagcagcaccagtttccgcagcaacagctgcctcagcaacagcagatcccacaacaacaacaaattcctcagcagccccagcaaatcccacagcagcagcaaattccccaacagccacagcaattccctcaacaacagttcccacagcaacaatttccacagcaacagttcccgcagcaagagttcccacagcagcaacagttccctcagcaacagatcgctcgtcagccacagcagctgccacaacagcagcaaattccacagcaaccccaacaattcccacaacaacaacagtttccgcaacagcagtctccccagcagcagcagtttccccagcagcagtttccacaacagcagcagctgcctcagaagcaattccctcagccacagcagatccctcagcagcagcagatccctcaacagccccaacagttcccgcagcagcagtttccccaacaacaacagttccctcagcagcaagagtttcctcaacagcaatttcctcagcaacaattccaccagcagcaactgccccaacaacaattcccgcaacagcaattcccgcagcagcaatttcctcagcaacagcaattcccacaacagcagcaactgacccagcaacagtttcctcgtcctcagcagtcccctgaacaacagcagtttccacagcagcaattcccgcaacagcctcctcagcagttcccgcaacaacagtttcctattccttatcctcctcagcaatccgaggaaccctcaccttaccagcagtacccacagcaacagccctccggttccgacgtgatctccatctctggtctgtaa

SEQ ID NO: 89 - DNA, 720 bp, Artificial Sequence
  Tri a 19 short
atgcaagtggtgctgggttccctgttcctgctgctgctgtctacctctcacggctggcaaatccgtgaccgtatcggcgacaacgagctggaagagaggggatcccaccaccaccatcaccacggttccggactgaacgacatcttcgaggctcaaaagatcgagtggcacgagggctcgagccaacaacagtttccgcaacagcagtctccccagcagcagcagtttccccagcagcagtttccacaacagcagcagctgcctcagaagcaattccctcagccacagcagatccctcagcagcagcagatccctcaacagccccaacagttcccgcagcagcagtttccccaacaacaacagttccctcagcagcaagagtttcctcaacagcaatttcctcagcaacaattccaccagcagcaactgccccaacaacaattcccgcaacagcaattcccgcagcagcaatttcctcagcaacagcaattcccacaacagcagcaactgacccagcaacagtttcctcgtcctcagcagtcccctgaacaacagcagtttccacagcagcaattcccgcaacagcctcctcagcagttcccgcaacaacagtttcctattccttatcctcctcagcaatccgaggaaccctcaccttaccagcagtacccacagcaacagccctccggttccgacgtgatctccatctctggtctgtaa

SEQ ID NO: 90 - DNA, 1716 bp, Artificial Sequence
  HA_A_Brisbane-2018
atgaaggccatcctggtggtgctgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctcggcggagttgctcctctgcacctgggcaagtgtaatatcgccggctggattctgggcaaccccgagtgtgaaagcctgagcacagccagatcctggtcctacatcgtggaaaccagcaacagcgacaacggcacatgcttccccggcgacttcatcaactacgaggaactgcgggaacagctgagcagcgtgtccagcttcgagagattcgagatcttccccaagaccagcagctggcccaaccacgattctaacaagggcgtgacagccgcctgtcctcatgccggcgctaagagcttctacaagaacctgatctggctcgtgaagaagggcaacagctaccccaagctgaaccagacctacatcaacgacaagggcaaagaggtgctggtcctctggggcatccaccatccacctacaacagccgatcagcagagcctgtaccagaacgccgatgcctatgtgttcgtgggcaccagccggtacagcaagaagttcaagcccgagatcgccaccaggcctaaagtgcgggatagagaaggccggatgaactactactggaccctggtggaacccggcgacaagatcacatttgaggccaccggcaacctggtggtccccagatacgccttcaccatggaaagaaatgccggcagcggcatcatcatcagcgacacccctgtgcacgactgcaacaccacttgtcagacagccgagggcgccatcaataccagcctgcctttccagaacgtgcaccccgtgaccatcggcaagtgccccaaatacgtgaagtccaccaagctgagactggccacaggcctgagaaacgtgccctccattcagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctcctggaaaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaaaaacggcacctacgactaccctaagtacagcgaggaagccaaactgaaccgcgagaagatcgacggctccggatatattcccgaggctcccagagatggccaggcctatgttcggaaggatggcgagtgggtcctgctgagcacattccttggatccggcctgaacgacatcttcgaggcccagaaaatcgagtggcacgagggccaccaccatcatcaccattga